(19) United States
(12) Patent Application Publication
(10) Pub. No.: US 2024/0209094 A1
(43) Pub. Date: Jun. 27, 2024

(54) NEW STABLE ANTI-VISTA ANTIBODY

(71) Applicant: PIERRE FABRE MEDICAMENT, Lavaur (FR)

(72) Inventor: Alain BECK, Feigeres (FR)

(73) Assignee: PIERRE FABRE MEDICAMENT, Lavaur (FR)

(21) Appl. No.: 18/288,634

(22) PCT Filed: May 2, 2022
(86) PCT No.: PCT/EP2022/061718

§ 371 Date: Oct. 27, 2023

Related U.S. Application Data
(60) Provisional application No. 63/182,316, filed on Apr. 30, 2021.

(51) Int. Cl. C07K 16/28; A61K 39/00; A61K 39/395; A61K 47/68; A61P 35/00

(52) U.S. Cl. CPC C07K 16/2827; A61K 39/3955; A61K 47/6849; A61P 35/00; A61K 2039/505

(57) ABSTRACT

The present invention provides an anti-VISTA antibody which is suitable for pharmaceutical development, as pharmaceutical compositions comprising this antibody, and methods of treating VISTA-mediated diseases.

### INTRODUCTION

Development of a therapeutic antibody is a complex process. Various physiochemical and functional liabilities can compromise the production or the therapeutic efficacy of antibodies. The present disclosure provides an anti-VISTA antibody which is suitable for pharmaceutical development, as pharmaceutical compositions comprising this antibody, and methods of treating VISTA-mediated diseases.

Monoclonal antibodies are a major class of bio-pharmaceuticals with indications now covering a large panel of diseases, from cancer to asthma, including central nervous system disorders, infectious diseases and cardiovascular diseases. Chemical stability is a major concern in the development of protein therapeutics due to its impact on both efficacy and safety and is linked to numerous factors such as formulation, environment, manipulations, as well as the protein own structure. Antibody drugs display a wide range of minor chemical changes, including glycan structural differences, asparagine (Asn) deamidation, aspartate (Asp) isomerisation, methionine/tryptophan (Met/Trp) oxidation, and non-enzymatic lysine (Lys) glycation, some of which may affect the safety or efficacy of the drugs. In particular, it is known that degradation of Asn and Asp residues can affect in vitro stability and in vivo biological functions. While these reactions may be kept under control by appropriate storage and formulation conditions of the final antibody drug product, degradation during fermentation, downstream-processing, and in vivo cannot be controlled sufficiently, leading to potential loss of potency and/or increased clearance.

Asn deamidation is a very common non-enzymatic modification affecting recombinant monoclonal antibodies. The side-chain carbonyl group of Asn is vulnerable to the nucleophilic attack by the nitrogen of the n+1 peptide bond, resulting in formation of a metastable cyclic succinimide intermediate. The succinimide intermediate is then hydrolysed to either Asp (a peptide linkage) or iso-Asp (B peptide linkage) end products. In monoclonal antibodies, deamidation has been reported in the Fc regions and the complementary-determining regions (CDRs). Deamidation in the CDR region could affect drug efficacy. For example, various studies have reported that CDR deamidation may exert a direct effect on target binding; see e.g., Harris et al. J Chromatogr B Biomed Sci Appl. 752(2): 233-245 (2001); Vlasak et al. Anal Biochem. 392(2): 145-154 (2009); Yan et al. J Pharm Sci. 98(10): 3509-3521 (2009); Yang et al. mAbs. 5(5): 787-794 (2013). Strikingly, the replacement of Asn33 with an Asp residue in a human anti-CD52 IgG1, thus mimicking a deamidation product, led to a 400-fold decrease in antigen binding affinity (Qiu et al. mAbs. 11(7): 1266-1275 (2019)).

Immunotherapy has been a game-changer in the field of cancer therapy. In order to ensure that an immune inflammatory response is not constantly activated once tumour antigens have stimulated a response, multiple controls or “checkpoints” are in place or activated. VISTA (V-Domain Ig Suppressor of T Cell Activation) is a negative checkpoint control protein that regulates T cell activation and immune responses. VISAT is a member of the B7 family which comprises several immune checkpoint proteins such as PD-L1. However, unlike of the members of this family, VISTA comprises a single unusually large Ig-like V-type domain. In addition, VISTA cytoplasmic tail domain contains several docketing sites for effector proteins, suggesting that VISTA could potentially function as both a receptor and a ligand.

Human VISTA has two confirmed binding partners with immunosuppressive functions, PSGL-1 and VSIG3. VISTA interacts with VSIG3 at physiological pH, but at acidic pH VISTA-expressing cells can bind to PSGL-1 on T cells (Wang et al. Immunology. 156(1): 74-85 (2019); Johnston et al. Nature. 574(7779): 565-570 (2019)). Both interactions result in inhibition of T cell function. Other receptors, including VSIG8 (WO 2016/090347A1) and LRIG1 (WO 2015/187359, have also been reported.

Physiologically, VISTA exerts a regulatory function on the immune system at several levels, particularly by modulating T cells activation. More recently, VISTA was identified as the earliest checkpoint regulator of peripheral T cell tolerance, particularly in the maintenance of naïve T cell quiescence. In the context of cancer, VISTA is upregulated on immunosuppressive tumour infiltrating leukocytes such as inhibitory regulatory T cells (Tregs) and myeloid-derived suppressor cells (MDSCs). The presence of VISTA in the tumour microenvironment hinders effective T cell responses and has been implicated in a number of human cancers including prostate, colon, skin, pancreatic, and lung.

Several antagonistic anti-VISTA antibodies have been described which can be used for the treatment of cancer (ElTanbouly et al. Clin Exp Immunol. 200(2): 120-130 (2020); Mehta et al. Sci Rep. 10(1):1 5171 (2020); Yuan et al. Trends Immunol. 42(3): 209-227 (2021); Tagliamento et al. Immunotargets Ther. 10: 185-200 (2021); Thakkar et al. J Immunother Cancer. 10(2): e003382 (2022); WO 2015/097536; WO 2016/094837; WO 2017/181139; WO 2019/183040). In particular, WO 2016/094837 discloses an antibody capable of inhibiting VISTA suppression of the anti-tumour immune response, thereby conferring protective anti-tumour immunity. However, this antibody comprises several potential Asn residues potentially susceptible to deamidation which could thus affect drug efficacy and clinical and manufacturing development. Thus there is need for a homogenous, safe and efficacious anti-VISTA antibody.

All methods and materials similar or equivalent to those described herein can be used in the practice or testing of the present invention, with suitable methods and materials being described herein. The practice of the invention employs, unless other otherwise indicated, conventional techniques or protein chemistry, molecular virology, microbiology, recombinant DNA technology, and pharmacology, which are within the skill of the art. Such techniques are explained fully in the literature (see e.g., Ausubel et al., Short Protocols in Molecular Biology, Current Protocols; 5th Ed., 2002; Remington's Pharmaceutical Sciences, 17th ed., Mack Publishing Co., Easton, Pa., 1985; and Sambrook et al., Molecular Cloning: A Laboratory Manual, Cold Spring Harbor Laboratory Press; 3rd Ed., 2001). The nomenclatures used in connection with, and the laboratory procedures and techniques of, molecular and cellular biology, protein biochemistry, enzymology and medicinal and pharmaceutical chemistry described herein are those well-known and commonly used in the art. All publications, patent applications, patents, and other references mentioned herein are incorporated by reference in their entirety. Further, the materials, methods, and examples are illustrative only and are not intended to be limiting, unless otherwise specified.

Other features and advantages of the invention will be apparent from the following detailed description, and from the claims.

### SUMMARY

The following aspects are provided herein:

In a first aspect the present disclosure provides an isolated antibody, or antigen-binding fragment thereof, which specifically binds VISTA. This antibody has the heavy chain and light chains provided herein. In particular, the present anti-VISTA antibody has an aspartic acid at position 55. The anti-VISTA antibody disclosed herein is preferably not susceptible to deamidation.

Preferably, the antibody is a monoclonal antibody, more preferably a humanised antibody.

In another aspect, the antibody is conjugated to a cytotoxic agent to provide an antibody-drug conjugate.

In another aspect, the present disclosure provides a polynucleotide comprising a nucleotide sequence encoding the heavy chain of the monoclonal anti-VISTA antibody provided herein. The present disclosure also provides a polynucleotide comprising a nucleotide sequence encoding the light chain of the monoclonal anti-VISTA antibody provided herein. The present disclosure also provides a polynucleotide comprising a nucleotide sequence encoding the heavy chain and the light chain of the monoclonal anti-VISTA antibody provided herein.

In another aspect, the present disclosure provides an expression vector comprising at least one of the polynucleotides provided herein.

In another aspect, the present disclosure provides a host cell comprising said expression vector.

In another aspect, the present disclosure provides a method of producing a monoclonal anti-VISTA antibody as provided herein, said method comprising a step of culturing the host cell provided herein under suitable conditions; and a step of recovering the anti-VISTA antibody, from the culture medium or from the cultured cells.

In another aspect, the present disclosure provides a pharmaceutical composition comprising the anti-VISTA antibody or the conjugate thereof, and a pharmaceutically acceptable diluent, carrier or excipient. The pharmaceutical composition may comprise a buffering agent, preferably a citrate buffer, a phosphate buffer, or a histidine buffer, more preferably a histidine buffer. The pharmaceutical composition may also comprise tonicity modifier. Preferably, the tonicity modifier is selected in the group consisting of polyhydric sugar alcohols, for example trihydric or higher sugar alcohols, such as glycerin, erythritol, arabitol, xylitol, sorbitol and mannitol, salts and amino acids; more preferably, a salt selected in the group consisting of sodium chloride, sodium succinate, sodium sulphate, potassium chloride, magnesium chloride, magnesium sulphate, and calcium chloride; even more preferably NaCl, MgCl₂, and/or CaCl₂). The pharmaceutical composition may comprise a non-ionic surfactant, preferably a polysorbate, e.g., Polysorbate 20 or Polysorbate 80. Preferably, the pharmaceutical composition comprises 25 mM Histidine, 150 mM NaCl, 0.3% Polysorbate 80 (w/w), pH 6.5, in addition to the monoclonal anti-VISTA antibody disclosed herein.

In another aspect, the monoclonal anti-VISTA antibody or the immunoconjugate or the pharmaceutical composition disclosed herein are for use in the treatment of a VISTA-mediated disease, notably a cancer, in a patient. Preferably, this use comprises inducing an immune response in the patient. Preferably, the immune response includes induction of CD4+ T cell proliferation, induction of CD8+ T cell proliferation, induction of CD4+ T cell cytokine production, and induction of CD8+ T cell cytokine production. Preferably, the use disclosed herein comprises activation of the effector functions of the antibody.

In a preferred aspect, the therapeutic use disclosed herein comprises the administration of a second therapeutic agent. This second therapeutic agent is advantageously an anti-PD-1 antibody or an anti-PD-L1 antibody.

Preferably, the cancer is selected from bladder cancer, breast cancer, cervical cancer, colon cancer, endometrial cancer, oesophageal cancer, fallopian tube cancer, gall bladder cancer, gastrointestinal cancer, head-and-neck cancer, haematological cancer (e.g., leukaemia, lymphoma, or myeloma), laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma, and prostate cancer.

In yet another aspect, the present disclosure provides an in vitro method for detecting a VISTA-mediated cancer in a subject, the method comprising the steps of contacting a biological sample of the subject with the monoclonal anti-VISTA antibody as provided herein; and detecting the binding of the antibody with the biological sample, wherein the binding of the anti-VISTA antibody indicates the presence of a VISTA-mediated cancer. Preferably, the monoclonal anti-VISTA antibody is labelled with a detectable label.

### FIGURE LEGENDS

FIG. 1: Charge variants identified with cation exchange chromatography, pH gradient. Left panel: Several peaks are visible for product 1, comprising Ab3, showing that it is subject to degradation, specifically deamidation at amino acid residue 55. Right panel: A singly peak is visible for product 2, comprising Ab1, showing that it is no longer susceptible to deamidation at amino acid residue 55, and is stable.

FIG. 2: Graphic representation of averaged data obtained with antibodies of the third series in the three experiments (N=3) in direct rhVISTA ELISA. Filed square: Ab1, diamond: Ab3 batch 1, inverted triangle: Ab3 batch 2, triangle: IgG1 anti-VISTA (positive control), open circles: c9G4 (negative control), open circles and dotted line: anti-hVISTA polyclonal antibody (positive control).

FIG. 3: Graphic representation of averaged data obtained with antibodies of the third series in the three experiments (N=3) in indirect rhVISTA ELISA. Filed square: Ab1, diamond: Ab3 batch 1, inverted triangle: Ab3 batch 2, triangle: IgG1 anti-VISTA (positive control), open circles: c9G4 (negative control), open circles and dotted line: anti-hVISTA polyclonal antibody (positive control).

FIG. 4: Evaluation of T cells activation and cytokines release in CHO-VISTA coculture with PBMC: schematic representation of the experiment.

FIG. 5: Evaluation of T cells activation and cytokines release in CHO-VISTA coculture with PBMC: Anti-VISTA Ab1 competent induced T cells activation and cytokines release in CHO-VISTA coculture with PBMC. (Donor 119). Ab1 silent: Ab1 variant with the N298A mutation.

FIG. 6: Binding of rhVISTA-Fc and rhVISTA-TagHis on rhVSIG3-Fc=f([anti-VISTA Ab1]).

FIG. 7: In vivo activity of the competent anti-VISTA Ab1 in a MC38 xenograft model.

FIG. 8: In vivo activity of the silent anti-VISTA Ab1 (N298A variant) in a MC38 xenograft model.

### DETAILED DESCRIPTION

The present invention will become more fully understood from the detailed description given herein and from the accompanying drawings, which are given by way of illustration only and do not limit the intended scope of the invention.

### Definitions

Unless specifically defined, all technical and scientific terms used herein have the same meaning as commonly understood by a skilled artisan in chemistry, biochemistry, cellular biology, molecular biology, and medical sciences.

The term “about” or “approximately” refers to the normal range of error for a given value or range known to the person of skills in the art. It usually means within 20%, such as within 10%, or within 5% (or 1% or less) of a given value or range.

As used herein, the expression “Antibody-dependent cell-mediated cytotoxicity”, “Antibody-dependent cellular cytotoxicity” or “ADCC” refers to a form of cytotoxicity in which an immunoglobulin bound onto Fc receptors (FcRs) present on certain cytotoxic effector cells enables these cytotoxic effector cells to bind specifically to an antigen-bearing target cell and subsequently kill the target cell with cytotoxins. Lysis of the target cell is extracellular, requires direct cell-to-cell contact, and does not involve complement. Cell destruction can occur, for example, by lysis or phagocytosis. To assess ADCC activity of a molecule of interest, an in vitro ADCC assay, such as that described in U.S. Pat. No. 5,500,362 or 5,821,337 may be performed. Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al, PNAS (USA) 95:652-656 (1998).

“Antibody-dependent phagocytosis” or “ADCP” or “opsonisation” as used herein refers to the cell-mediated reaction wherein nonspecific cytotoxic cells that express FcyRs recognise bound antibody on a target cell and subsequently cause phagocytosis of the target cell.

As used herein, “administer” or “administration” refers to the act of injecting or otherwise physically delivering a substance as it exists outside the body (e.g., an anti-VISTA antibody provided herein) into a patient, such as by mucosal, intradermal, intravenous, intramuscular delivery and/or any other method of physical delivery described herein or known in the art. When a disease, or a symptom thereof, is being treated, administration of the substance typically occurs after the onset of the disease or symptoms thereof. When a disease, or symptoms thereof, are being prevented, administration of the substance typically occurs before the onset of the disease or symptoms thereof.

As used herein, an “antagonist” or “inhibitor” refers to a molecule that is capable of inhibiting or otherwise decreasing one or more of the biological activities of a target protein, such as VISTA. In some embodiments, an antagonist of VISTA (e.g., an antagonistic antibody provided herein) can, for example, act by inhibiting or otherwise decreasing the activation and/or cell signalling pathways of the cell expressing VISTA (e.g., a VISTA-bearing tumour cell, a regulatory T cell, a myeloid-derived suppressor cell or a suppressive dendritic cell), thereby inhibiting a biological activity of the cell relative to the biological activity in the absence of the antagonist. For example, an antagonist of VISTA may inhibit VISTA's suppressive effects on T cell immunity (CD4+ and/or CD8+ T cell immunity) and/or the expression of proinflammatory cytokines. More specifically, an antagonist of VISTA may block or decrease the interaction of VISTA with at least one of its ligands, including VSIG3, PSG-L1, VSIG8, and LRIG1. Even more specifically, an antagonist of VISTA may block or decrease the interaction of VISTA with either of VSIG3 or PSG-L1. Preferably, an antagonist of VISTA may block or decrease the interaction of VISTA with PSG-L1 at acidic pH (i.e., at pH between 5.9 and 6.5). In some embodiments the antibodies provided herein are antagonistic anti-VISTA-1 antibodies. Certain antagonistic antibodies substantially or completely inhibit one or more of the biological activities of said antigen. For example, an antagonistic anti-VISTA antibody may inhibit VISTA's suppressive effects on T cell immunity (CD4+ and/or CD8+ T cell immunity) and/or the expression of proinflammatory cytokines. More specifically, an antagonistic anti-VISTA antibody may block or decrease the interaction of VISTA with at least one of its ligands, including VSIG3, PSG-L1, VSIG8, and LRIG1. Even more specifically, an antagonistic anti-VISTA antibody may block or decrease the interaction of VISTA with either of VSIG3 or PSG-L1. Preferably, an antagonistic anti-VISTA antibody may block or decrease the interaction of VISTA with PSG-L1 at acidic pH (i.e., at pH between 5.9 and 6.5).

The terms “antibody” and “immunoglobulin” or “Ig” are used interchangeably herein. These terms are used herein in the broadest sense and specifically cover monoclonal antibodies (including full length monoclonal antibodies) of any isotype such as IgG, IgM, IgA, IgD, and IgE, polyclonal antibodies, multispecific antibodies, chimeric antibodies, and antibody fragments, provided that said fragments retain the desired biological function. These terms are intended to include a polypeptide product of B cells within the immunoglobulin class of polypeptides that is capable of binding to a specific molecular antigen and is composed of two identical pairs of polypeptide chains inter-connected by disulfide bonds, wherein each pair has one heavy chain (about 50-70 kDa) and one light chain (about 25 kDa) and each amino-terminal portion of each chain includes a variable region of about 100 to about 130 or more amino acids and each carboxy-terminal portion of each chain includes a constant region (See, Borrebaeck (ed.) (1995) Antibody Engineering, Second Ed., Oxford University Press.; Kuby (1997) Immunology, Third Ed., W.H. Freeman and Company, New York). Each variable region of each heavy and light chain is composed of three complementarity-determining regions (CDRs), which are also known as hypervariable regions and four frameworks (FRs), the more highly conserved portions of variable domains, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. In some embodiments, the specific molecular antigen can be bound by an antibody provided herein includes the target VISTA polypeptide, fragment or epitope. An antibody reactive with a specific antigen can be generated by recombinant methods such as selection of libraries of recombinant antibodies in phage or similar vectors, or by immunising an animal with the antigen or an antigen-encoding nucleic acid.

Antibodies also include, but are not limited to, synthetic antibodies, monoclonal antibodies, recombinantly produced antibodies, multispecific antibodies (including bi-specific antibodies), human antibodies, humanised antibodies, camelised antibodies, chimeric antibodies, intrabodies, anti-idiotypic (anti-Id) antibodies, and functional fragments of any of the above, which refers a portion of an antibody heavy or light chain polypeptide that retains some or all of the biological function of the antibody from which the fragment was derived. The antibodies provided herein can be of any type (e.g., IgG, IgE, IgM, IgD, IgA and IgY), any class (e.g., IgG1, IgG2, IgG3, IgG4, IgA1 and IgA2), or any subclass (e.g., IgG2a and IgG2b) of immunoglobulin molecule.

The terms “anti-VISTA antibodies” “antibodies that bind to VISTA,” “antibodies that bind to a VISTA epitope,” and analogous terms are used interchangeably herein and refer to antibodies that bind to a VISTA polypeptide, such as a VISTA antigen or epitope. Such antibodies include polyclonal and monoclonal antibodies, including chimeric, humanised, and human antibodies. An antibody that binds to a VISTA antigen may be cross-reactive with related antigens. In some embodiments, an antibody that binds to VISTA does not cross-react with other antigens such as e.g., other peptides or polypeptides belonging to the B7 superfamily. An antibody that binds to VISTA can be identified, for example, by immunoassays, BIAcore, or other techniques known to those of skill in the art. An antibody binds to VISTA, for example, when it binds to VISTA with higher affinity than to any cross-reactive antigen as determined using experimental techniques, such as radioimmunoassays (RIA) and enzyme-linked immunosorbent assays (ELISAs), for example, an antibody that specifically binds to VISTA. Typically, a specific or selective reaction will be at least twice background signal or noise and may be more than 10 times background. See, e.g., Paul, ed., 1989, Fundamental Immunology Second Edition, Raven Press, New York at pages 332-336 for a discussion regarding antibody specificity. In some embodiments, an antibody “which binds” an antigen of interest is one that binds the antigen with sufficient affinity such that the antibody is useful as a diagnostic and/or therapeutic agent in targeting a cell or tissue expressing the antigen, and does not significantly cross-react with other proteins. In such embodiments, the extent of binding of the antibody to a “non-target” protein will be less than about 10% of the binding of the antibody to its particular target protein as determined by fluorescence activated cell sorting (FACS) analysis or radioimmunoprecipitation (RIPA). With regard to the binding of an antibody to a target molecule, the term “specific binding” or “specifically binds to” or is “specific for” a particular polypeptide or an epitope on a particular polypeptide target means binding that is measurably different from a non-specific interaction. Specific binding can be measured, for example, by determining binding of a molecule compared to binding of a control molecule, which generally is a molecule of similar structure that does not have binding activity. For example, specific binding can be determined by competition with a control molecule that is similar to the target, for example, an excess of non-labelled target. In this case, specific binding is indicated if the binding of the labelled target to a probe is competitively inhibited by excess unlabelled target. The term “specific binding” or “specifically binds to” or is “specific for” a particular polypeptide or an epitope on a particular polypeptide target as used herein can be exhibited, for example, by a molecule having a K_{D} for the target of at least about 10⁻⁴ M, alternatively at least about 10⁻⁵ M, alternatively at least about 10⁻⁶ M, alternatively at least about 10⁻⁷ M, alternatively at least about 10⁻⁸ M, alternatively at least about 10⁻⁹ M, alternatively at least about 10⁻¹⁰ M, alternatively at least about 10⁻¹¹ M, alternatively at least about 10⁻¹² M, or greater. In some embodiments, the term “specific binding” refers to binding where a molecule binds to a particular polypeptide or epitope on a particular polypeptide without substantially binding to any other polypeptide or polypeptide epitope. In some embodiments, an antibody that binds to VISTA has a dissociation constant (K_{D}) of ≤1 μM, ≤100 nM, ≤10 nM, ≤1 nM, or ≤0.1 nM.

An “antigen” is a predetermined antigen to which an antibody can selectively bind. The target antigen may be a polypeptide, carbohydrate, nucleic acid, lipid, hapten or other naturally occurring or synthetic compound. In some embodiments, the target antigen is a polypeptide, including, for example, a VISTA polypeptide.

The term “antigen binding fragment,” “antigen binding domain,” “antigen binding region,” and similar terms refer to that portion of an antibody which comprises the amino acid residues that interact with an antigen and confer on the binding agent its specificity and affinity for the antigen (e.g., the complementarity determining regions (CDRs)).

The term “antigen binding fragment,” “antigen binding domain,” “antigen binding region,” and similar terms refer to that portion of an antibody which comprises the amino acid residues that interact with an antigen and confer on the binding agent its specificity and affinity for the antigen (e.g., the complementarity determining regions (CDRs)). By the expression “antigen-binding fragment” of an antibody, it is intended to indicate any peptide, polypeptide, or protein retaining the ability to bind to the target (also generally referred to as antigen) of the said antibody, generally the same epitope, and comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, at least 150 contiguous amino acid residues, at least 175 contiguous amino acid residues, or at least 200 contiguous amino acid residues, of the amino acid sequence of the antibody. In a particular embodiment, the said antigen-binding fragment comprises at least one CDR of the antibody from which it is derived. Still in a preferred embodiment, the said antigen binding fragment comprises 2, 3, 4 or 5 CDRs, more preferably the 6 CDRs of the antibody from which it is derived.

The “antigen-binding fragments” can be selected, without limitation, in the group consisting of Fab, Fab′, (Fab′)₂, Fv, scFv (sc for single chain), Bis-scFv, scFv-Fc fragments, Fab2, Fab3, minibodies, diabodies, triabodies, tetrabodies, and nanobodies, and fusion proteins with disordered peptides such as XTEN (extended recombinant polypeptide) or PAS motifs, and any fragment of which the half-life time would be increased by chemical modification, such as the addition of poly(alkylene) glycol such as poly(ethylene) glycol (“PEGylation”) (pegylated fragments called Fv-PEG, scFv-PEG, Fab-PEG, F(ab′)₂-PEG or Fab′-PEG) (“PEG” for Poly(Ethylene) Glycol), or by incorporation in a liposome, said fragments having at least one of the characteristic CDRs of the antibody according to the invention. Among the antibody fragments, Fab has a structure including variable regions of light chain and heavy chain, a constant region of a light chain, and the first constant region of a heavy chain (CH1), and it has one antigen binding site. Fab′ is different from Fab in that it has a hinge region including one or more cysteine residues at C terminus of heavy chain CH1 domain. F(ab′)2 antibody is generated as the cysteine residues of the hinge region of Fab′ form a disulfide bond. Fv is a minimum antibody fragment which has only a heavy chain variable region and a light chain variable region, and a recombination technique for producing the Fv fragment is described in International Publication WO 88/10649 or the like. In double chain Fv (dsFv), the heavy chain variable region and light chain variable region are linked to each other via a disulfide bond, and, in single chain Fv (scFv), the heavy chain variable region and light chain variable region are covalently linked to each other via a peptide linker in general. Those antibody fragments can be obtained by using a proteinase (e.g., Fab can be obtained by restriction digestion of whole antibody with papain, and F(ab′)2 fragment can be obtained by restriction digestion with pepsin), and it can be preferably produced by genetic engineering techniques. Preferably, said “antigen-binding fragments” will be constituted or will comprise a partial sequence of the heavy or light variable chain of the antibody from which they are derived, said partial sequence being sufficient to retain the same specificity of binding as the antibody from which it is descended and a sufficient affinity, preferably at least equal to 1/100, in a more preferred manner to at least 1/10, of the affinity of the antibody from which it is descended, with respect to the target. Such antibody fragments can be found described in, for example, Harlow and Lane, Antibodies: A Laboratory Manual, Cold Spring Harbor Laboratory, New York (1989); Myers (ed.), Molec. Biology and Biotechnology: A Comprehensive Desk Reference, New York: VCH Publisher, Inc.; Huston et al., Cell Biophysics, 22:189-224 (1993); Plückthun and Skerra, Meth. Enzymol., 178:497-515 (1989) and in Day, E.D., Advanced Immunochemistry, Second Ed., Wiley-Liss, Inc., New York, NY (1990).

The term “antigen-presenting cell” or “APC” refers to a heterogeneous group of immune cells that mediate the cellular immune response by processing and presenting antigens for recognition by certain lymphocytes, such as T cells. APCs include, but are not limited to, dendritic cells, macrophages, Langerhans cells and B cells.

The terms “binds” or “binding” as used herein refer to an interaction between molecules to form a complex which, under physiologic conditions, is relatively stable. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions or forces. The strength of the total non-covalent interactions between a single antigen-binding site on an antibody and a single epitope of a target molecule, such as VISTA, is the affinity of the antibody or functional fragment for that epitope. The ratio of association (k₁) to dissociation (k₋₁) of an antibody to a monovalent antigen (k₁/k₋₁) is the association constant K, which is a measure of affinity. The value of K varies for different complexes of antibody and antigen and depends on both k₁ and k₋₁. The association constant K for an antibody provided herein can be determined using any method provided herein or any other method well known to those skilled in the art. The affinity at one binding site does not always reflect the true strength of the interaction between an antibody and an antigen. When complex antigens containing multiple, repeating antigenic determinants, such as a polyvalent VISTA, come in contact with antibodies containing multiple binding sites, the interaction of antibody with antigen at one site will increase the probability of a reaction at a second site. The strength of such multiple interactions between a multivalent antibody and antigen is called the avidity. The avidity of an antibody can be a better measure of its binding capacity than is the affinity of its individual binding sites. For example, high avidity can compensate for low affinity as is sometimes found for pentameric IgM antibodies, which can have a lower affinity than IgG, but the high avidity of IgM, resulting from its multivalence, enables it to bind antigen effectively. Methods for determining whether two molecules bind are well known in the art and include, for example, equilibrium dialysis, surface plasmon resonance, and the like. In a particular embodiment, said antibody, or antigen-binding fragment thereof, binds to VISTA with an affinity that is at least two-fold greater than its affinity for binding to a non-specific molecule such as BSA or casein. In a more particular embodiment, said antibody, or antigen-binding fragment thereof, binds only to VISTA.

As used herein, the term “biological sample” or “sample” refers to a sample that has been obtained from a biological source, such as a patient or subject. A “biological sample” as used herein refers notably to a whole organism or a subset of its tissues, cells or component parts (e.g., blood vessel, including artery, vein and capillary, body fluids, including but not limited to blood, serum, mucus, lymphatic fluid, synovial fluid, cerebrospinal fluid, saliva, amniotic fluid, amniotic cord blood, urine, vaginal fluid and semen). “Biological sample” further refers to a homogenate, lysate or extract prepared from a whole organism or a subset of its tissues, cells or component parts, or a fraction or portion thereof. Lastly, “biological sample” refers to a medium, such as a nutrient broth or gel in which an organism has been propagated, which contains cellular components, such as proteins or nucleic acid molecules.

e.g. The terms “cell proliferative disorder” and “proliferative disorder” refer to disorders that are associated with some degree of abnormal cell proliferation. In some embodiments, the cell proliferative disorder is a tumour or cancer. “Tumour,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumour” are not mutually exclusive as referred to herein. The terms “cancer” and “cancerous” refer to or describe the physiological condition in mammals that is typically characterised by unregulated cell growth. A “cancer” as used herein is any malignant neoplasm resulting from the undesired growth, the invasion, and under certain conditions metastasis of impaired cells in an organism. The cells giving rise to cancer are genetically impaired and have usually lost their ability to control cell division, cell migration behaviour, differentiation status and/or cell death machinery. Most cancers form a tumour but some hematopoietic cancers, such as leukaemia, do not. Thus, a “cancer” as used herein may include both benign and malignant cancers. The term “cancer” as used herein refers in particular to any cancer that can be treated by the human antibody of the present disclosure without any limitation. Examples of cancer include, but are not limited to, carcinoma, lymphoma, blastoma, sarcoma, and leukaemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, oral cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, multiple myeloma and B-cell lymphoma, brain cancer, as well as head and neck cancer, and associated metastases. In some embodiments, the cancer is a haematological cancer, which refers to cancer that begins in blood-forming tissue, such as the bone marrow, or in the cells of the immune system. Examples of a haematologic cancer are leukaemia (e.g., acute myeloid leukaemia (AML), acute lymphoblastic leukaemia (ALL), chronic myelogenous leukaemia (CML), chronic lymphocytic leukaemia (CLL), or acute monocytic leukaemia (AMOL)), lymphoma (Hodgkin lymphoma or non-Hodgkin lymphoma), and myeloma (multiple myeloma, plasmacytoma, localised myeloma or extramedullary myeloma).

A “chemotherapeutic agent” is a chemical or biological agent (e.g., an agent, including a small molecule drug or biologic, such as an antibody or cell) useful in the treatment of cancer, regardless of mechanism of action. Chemotherapeutic agents include compounds used in targeted therapy and conventional chemotherapy. Chemotherapeutic agents include, but are not limited to, alkylating agents, anti-metabolites, anti-tumour antibiotics, mitotic inhibitors, chromatin function inhibitors, anti-angiogenesis agents, anti-oestrogens, anti-androgens or immunomodulators.

As used herein, a “CDR” refers to one of three hypervariable regions (H1, H2 or H3) within the non-framework region of the immunoglobulin (Ig or antibody) VH β-sheet framework, or one of three hypervariable regions (L1, L2 or L3) within the non-framework region of the antibody VL β-sheet framework. Accordingly, CDRs are variable region sequences interspersed within the framework region sequences. CDR regions are well known to those skilled in the art and have been defined by, for example, Kabat as the regions of most hypervariability within the antibody variable (V) domains (Kabat et al. (1977) J. Biol. Chem. 252:6609-6616; Kabat (1978) Adv. Prot. Chem. 32:1-75). The Kabat CDRs are based on sequence variability and are the most commonly used (Kabat eta/. (1991) Sequences of Proteins of Immunological Interest, 5th Ed. Public Health Service, National Institutes of Health, Bethesda, MD). Chothia refers instead to the location of the structural loops (Chothia and Lesk (1987) J Mol. Biol. 196:901-917). CDR region sequences also have been defined structurally by Chothia as those residues that are not part of the conserved β-sheet framework, and thus are able to adopt different conformations (Chothia and Lesk (1987) J. Mol. Biol. 196:901-917). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). Both terminologies are well recognised in the art. CDR region sequences have also been defined by AbM, Contact and IMGT. The AbM hypervariable regions represent a compromise between the Kabat CDRs and Chothia structural loops, and are used by Oxford Molecular's AbM antibody modelling software. The “contact” hypervariable regions are based on an analysis of the available complex crystal structures. Recently, a universal numbering system has been developed and widely adopted, ImMunoGeneTics (IMGT) Information System® (Lefranc et al. (2003) Dev. Comp. Immunol. 27(1):55-77). The IMGT universal numbering has been defined to compare the variable domains whatever the antigen receptor, the chain type, or the species [Lefranc M.-P. (1997) Immunol. Today 18: 509; Lefranc M.-P. (1999) The Immunologist 7: 132-136]. In the IMGT universal numbering, the conserved amino acids always have the same position, for instance cysteine 23 (1st-CYS), tryptophan 41 (CONSERVED-TRP), hydrophobic amino acid 89, cysteine 104 (2nd-CYS), phenylalanine or tryptophan 118 (J-PHE or J-TRP). The IMGT universal numbering provides a standardised delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. As gaps represent unoccupied positions, the CDR-IMGT lengths (shown between brackets and separated by dots, e.g. [8.8.13]) become crucial information. The IMGT universal numbering is used in 2D graphical representations, designated as IMGT Colliers de Perles [Ruiz, M. and Lefranc, M.-P., Immunogenetics, 53: 857-883 (2002); Kaas, Q. and Lefranc, M.-P., Current Bioinformatics, 2: 21-30 (2007)], and in 3D structures in IMGT/3Dstructure-DB [Kaas, Q., Ruiz, M. and Lefranc, M.-P., T cell receptor and MHC structural data. Nucl. Acids. Res., 32: D208-D210 (2004)]. The positions of CDRs within a canonical antibody variable domain have been determined by comparison of numerous structures (Al-Lazikani et al., J. Mol. Biol. 273:927-948 (1997); Morea et al., Methods 20:267-279 (2000)). Because the number of residues within a hypervariable region varies in different antibodies, additional residues relative to the canonical positions are conventionally numbered with a, b, c and so forth next to the residue number in the canonical variable domain numbering scheme (Al-Lazikani et al., supra (1997)). Such nomenclature is similarly well known to those skilled in the art.

Hypervariable regions may comprise “extended hypervariable regions” as follows: 24-36 or 24-34 (L1), 46-56 or 50-56 (L2) and 89-97 or 89-96 (L3) in the VL and 26-35 or 26-35A (H1), 50-65 or 49-65 (H2) and 93-102, 94-1 02, or 95-102 (H3) in the VH. The variable domain residues are 25 numbered according to Kabat et al., supra, for each of these definitions. As used herein, the terms “HVR” and “CDR” are used interchangeably.

As used herein, a “checkpoint inhibitor” refers to a molecule, such as e.g., a small molecule, a soluble receptor, or an antibody, which targets an immune checkpoint and blocks the function of said immune checkpoint. More specifically, a “checkpoint inhibitor” as used herein is a molecule, such as e.g., a small molecule, a soluble receptor, or an antibody, that is capable of inhibiting or otherwise decreasing one or more of the biological activities of an immune checkpoint. In some embodiments, an inhibitor of an immune checkpoint protein (e.g., an antagonistic anti-VISTA antibody provided herein) can, for example, act by inhibiting or otherwise decreasing the activation and/or cell signalling pathways of the cell expressing said immune checkpoint protein (e.g., a T cell), thereby inhibiting a biological activity of the cell relative to the biological activity in the absence of the antagonist. Example of immune checkpoint inhibitors include small molecule drugs, soluble receptors, and antibodies.

The term “complement-dependent cytotoxicity” or “CDC” as used herein refers to the process of antibody-mediated complement activation resulting in the lysis of a cell according to the mechanism outlined above upon binding of the antibody to its antigen located on that cell. The complement activation pathway is initiated by the binding of the first component of the complement system (C1q) to a molecule (e.g., an antibody) complexed with a cognate antigen. To assess complement activation, a CDC assay, e.g., as described in Gazzano-Santaro et al., J. Immunol. Methods, 202:163 (1996), may be performed. In the art normal human serum is used as a complement source.

The term “constant region” or “constant domain” refers to a carboxy terminal portion of the light and heavy chain which is not directly involved in binding of the antibody to antigen but exhibits various effector function, such as interaction with the Fc receptor. The terms refer to the portion of an immunoglobulin molecule having a more conserved amino acid sequence relative to the other portion of the immunoglobulin, the variable domain, which contains the antigen binding site. The constant domain contains the CH1, CH2 and CH3 domains of the heavy chain and the CL domain of the light chain.

As described herein, a “cytotoxic agent” refers to an agent which, when administered to a subject, treats or prevents the development of cell proliferation, preferably the development of cancer in the subject's body, by inhibiting or preventing a cellular function and/or causing cell death. The cytotoxic agent that can be used in the present antibody-drug conjugate includes any agent, part thereof, or residue having cytotoxic effect or inhibitory effect on cell proliferation. Examples of such agents include (i) chemotherapeutic agent capable of functioning as a microtubulin inhibitor, a mitotic inhibitor, a topoisomerase inhibitor, or a DNA interchelator; (ii) protein toxin capable of functioning enzymatically; and (iii) radioisotopes (radioactive nuclide). The cytotoxic agent may be conjugated to an antibody, such as e.g., an anti-VISTA antibody, to form an immunoconjugate. Preferably, the cytotoxic agent is released from the antibody under specific conditions, e.g., under acidic conditions, thereby affecting therapeutically the target cells, e.g., by preventing the proliferation thereof or by displaying a cytotoxic effect.

The term “decreased”, as used herein, refers to the activity of a protein, e.g., VISTA, at least 1-fold (e.g., 1, 2, 3, 4, 5, 10, 20, 30, 40, 50, 60, 70, 80, 90, 100, 1000, 10,000-fold or more) lower than its reference value. “Decreased”, as it refers to the activity of a protein, e.g., VISTA, of a subject, signifies also at least 5% lower (e.g., 5%, 6%, 7%, 8%, 9%, 10%, 15%, 20%, 25%, 30%, 35%, 40%, 45%, 50%, 55%, 60%, 65%, 70%, 75%, 80%, 85%, 90%), 95%), 99%), or 100%) than the activity of the protein in the reference sample or with respect to the reference value for said protein. The term “decreased”, as used herein, also refers to the level of a biomarker, e.g., VISTA, of a subject at least 1-fold (e.g., 1, 2, 3, 4, 5, 10, 20, 30, 40, 50, 60, 70, 80, 90, 100, 1000, 10,000-fold or more) lower than its reference value. “Decreased”, as it refers to the level of a biomarker, e.g., VISTA, of a subject, signifies also at least 5% lower (e.g., 5%, 6%, 7%, 8%, 9%, 10%, 15%, 20%, 25%, 30%, 35%, 40%, 45%, 50%, 55%, 60%, 65%, 70%, 75%, 80%, 85%, 90%), 95%), 99%), or 100%) than the level in the reference sample or with respect to the reference value for said marker.

The term “detecting” as used herein encompasses quantitative or qualitative detection.

The term “detectable probe,” as used herein, refers to a composition that provides a detectable signal. The term includes, without limitation, any fluorophore, chromophore, radiolabel, enzyme, antibody or antibody fragment, and the like, that provide a detectable signal via its activity.

In the context of a polypeptide, the term “derivative” as used herein refers to a polypeptide that comprises an amino acid sequence of a VISTA polypeptide, a fragment of a VISTA polypeptide, or an antibody that binds to a VISTA polypeptide which has been altered by the introduction of amino acid residue substitutions, deletions or additions. The term “derivative” as used herein also refers to a VISTA polypeptide, a fragment of a VISTA polypeptide, or an antibody that binds to a VISTA polypeptide which has been chemically modified, e.g., by the covalent attachment of any type of molecule to the polypeptide. For example, but not by way of limitation, a VISTA polypeptide, a fragment of a VISTA polypeptide, or a VISTA antibody may be chemically modified, e.g., by glycosylation, acetylation, pegylation, phosphorylation, derivatisation by known protecting/blocking groups, proteolytic cleavage, linkage to a cellular ligand or other protein, etc. The derivatives are modified in a manner that is different from naturally occurring or starting peptide or polypeptides, either in the type or location of the molecules attached. Derivatives further include deletion of one or more chemical groups which are naturally present on the peptide or polypeptide. A derivative of a VISTA polypeptide, a fragment of a VISTA polypeptide, or a VISTA antibody may be chemically modified by chemical modifications using techniques known to those of skill in the art, including, but not limited to specific chemical cleavage, acetylation, formulation, metabolic synthesis of tunicamycin, etc. Further, a derivative of a VISTA polypeptide, a fragment of a VISTA polypeptide, or a VISTA antibody may contain one or more non-classical amino acids. A polypeptide derivative possesses a similar or identical function as a VISTA polypeptide, a fragment of a VISTA polypeptide, or a VISTA antibody described herein.

The term “diagnostic agent” refers to a substance administered to a subject that aids in the diagnosis of a disease. Such substances can be used to reveal, pinpoint, and/or define the localisation of a disease-causing process. In some embodiments, a diagnostic agent includes a substance that is conjugated to an antibody provided herein, that when administered to a subject or contacted to a sample from a subject, aids in the diagnosis of cancer, tumour formation, or any other VISTA-mediated disease, disorder or condition.

The term “detectable agent” refers to a substance that can be used to ascertain the existence or presence of a desired molecule, such as an antibody provided herein, in a sample or subject. A detectable agent can be a substance that is capable of being visualised or a substance that is otherwise able to be determined and/or measured (e.g., by quantitation).

The term “detecting” as used herein encompasses quantitative or qualitative detection.

As used herein, “diagnosis” or “identifying a subject having” refers to a process of identifying a disease, condition, or injury from its signs and symptoms. A diagnosis is notably a process of determining if an individual is afflicted with a disease or ailment (e.g., cancer). Cancer is diagnosed for example by detecting either the presence of a marker associated with cancer such as, e.g., VISTA.

An “effective amount” or “therapeutically effective amount” of an agent, e.g., a pharmaceutical formulation, refers to an amount effective, at dosages and for periods of time necessary, to elicit the desired biological response in a subject. Such response includes alleviation of the symptoms of the disease or disorder being treated, prevention, inhibition or a delay in the recurrence of symptom of the disease or of the disease itself, an increase in the longevity of the subject compared with the absence of the treatment, or prevention, inhibition or delay in the progression of symptom of the disease or of the disease itself. An “effective amount” is in particular the amount of the agent effective to achieve the desired therapeutic or prophylactic result. More specifically, an “effective amount” as used herein is an amount of the agent that confers a therapeutic benefit. A therapeutically effective amount is also one in which any toxic or detrimental effects of the agent are outweighed by the therapeutically beneficial effects.

An effective amount can be administered in one or more administrations, applications or dosages. Such delivery is dependent on a number of variables including the time period for which the individual dosage unit is to be used, the bioavailability of the agent, the route of administration, etc. In some embodiments, effective amount also refers to the amount of an antibody (e.g., an anti-VISTA antibody) provided herein to achieve a specified result (e.g., inhibition of an immune checkpoint biological activity, such as modulating T cell activation). In some embodiments, this term refers to the amount of a therapy (e.g., an immune checkpoint inhibitor such as e.g., an anti-VISTA antibody) which is sufficient to reduce and/or ameliorate the severity and/or duration of a given disease, disorder or condition and/or a symptom related thereto. This term also encompasses an amount necessary for the reduction or amelioration of the advancement or progression of a given disease, disorder or condition, reduction or amelioration of the recurrence, development or onset of a given disease, disorder or condition, and/or to improve or enhance the prophylactic or therapeutic effect(s) of another therapy (e.g., a therapy other than said immune checkpoint inhibitor). In the context of cancer therapy, a therapeutic benefit means for example any amelioration of cancer, including any one of, or combination of, halting or slowing the progression of cancer (e.g., from one stage of cancer to the next), halting or delaying aggravation or deterioration of the symptoms or signs of cancer, reducing the severity of cancer, inducing remission of cancer, inhibiting tumour cell proliferation, tumour size, or tumour number, or reducing levels of biomarker(s) indicative of the cancer. In some embodiments, the effective amount of an antibody is from about 0.1 mg/kg (mg of antibody per kg weight of the subject) to about 100 mg/kg. In some embodiments, an effective amount of an antibody provided therein is about 0.1 mg/kg, about 0.5 mg/kg, about 1 mg/kg, 3 mg/kg, 5 mg/kg, about 10 mg/kg, about 15 mg/kg, about 20 mg/kg, about 25 mg/kg, about 30 mg/kg, about 35 mg/kg, about 40 mg/kg, about 45 mg/kg, about 50 mg/kg, about 60 mg/kg, about 70 mg/kg, about 80 mg/kg about 90 mg/kg or about 100 mg/kg (or a range therein).

As used herein, the term “effector functions” refer to biological functions carried by the Fc domain of an immunoglobulin (e.g., the anti-VISTA antibody described herein). These Fc domain-mediated activities are mediated via immunological effector cells, such as killer cells, natural killer cells, and activated macrophages, or various complement components. These effector functions involve activation of receptors on the surface of said effector cells, through the binding of the Fc domain of an antibody to the said receptor or to complement component(s). “Effector functions” as used herein encompass such activities as antibody-dependent cell-mediated cytotoxicity (ADCC), antibody-dependent cellular phagocytosis (ADCP), complement-dependent cytotoxicity (CDC).

“Effector cells” as used herein refer to leukocytes which express one or more FcRs and perform effector functions. The cells express at least FcγRI, FCγRII, FcγRIII and/or FcγRIV and carry out ADCC effector function. FcR expression on hematopoietic cells is summarised in Table 3 on page 464 of Ravetch and Kinet, Annu. Rev. Immunol 9:457-92 (1991). Examples of human leukocytes which mediate ADCC include peripheral blood mononuclear cells (PBMC), natural killer (NK) cells, monocytes, cytotoxic T cells and neutrophils.

The term “encode” or grammatical equivalents thereof as it is used in reference to nucleic acid molecule refers to a nucleic acid molecule in its native state or when manipulated by methods well known to those skilled in the art that can be transcribed to produce mRNA, which is then translated into a polypeptide and/or a fragment thereof. The antisense strand is the complement of such a nucleic acid molecule, and the encoding sequence can be deduced therefrom.

The term “epitope” as used herein refers to the region of an antigen, such as VISTA polypeptide or VISTA polypeptide fragment, to which an antibody binds. Preferably, an epitope as used herein is a localised region on the surface of an antigen, such as VISTA polypeptide or VISTA polypeptide fragment, that is capable of being bound to one or more antigen binding regions of an antibody, and that has antigenic or immunogenic activity in an animal, such as a mammal (e.g., a human), that is capable of eliciting an immune response. An epitope having immunogenic activity is a portion of a polypeptide that elicits an antibody response in an animal. An epitope having antigenic activity is a portion of a polypeptide to which an antibody binds as determined by any method well known in the art, for example, by an immunoassay. Antigenic epitopes need not necessarily be immunogenic. Epitopes usually consist of chemically active surface groupings of molecules such as amino acids or sugar side chains and have specific three-dimensional structural characteristics as well as specific charge characteristics. An epitope can be formed by contiguous residues or by non-contiguous residues brought into close proximity by the folding of an antigenic protein. Epitopes formed by contiguous amino acids are typically retained on exposure to denaturing solvents, whereas epitopes formed by non-contiguous amino acids are typically lost under said exposure. In some embodiments, a VISTA epitope is a three-dimensional surface feature of a VISTA polypeptide. In other embodiments, a VISTA epitope is linear feature of a VISTA polypeptide. Generally, an antigen has several or many different epitopes and reacts with many different antibodies.

The term “excipient” as used herein refers to an inert substance which is commonly used as a diluent, vehicle, preservative, binder, or stabilising agent for drugs which imparts a beneficial physical property to a formulation, such as increased protein stability, increased protein solubility, and decreased viscosity. Examples of excipients include, but are not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, non-ionic surfactant, etc.), saccharides (e.g., sucrose, maltose, trehalose, etc.) and polyols (e.g., mannitol, sorbitol, etc.). See, also, Remington's Pharmaceutical Sciences (1990) Mack Publishing Co., Easton, PA, which is hereby incorporated by reference in its entirety.

The term “framework” or “FR” residues refers to those variable domain residues other than the hypervariable region residues herein defined. FR residues are those variable domain residues flanking the CDRs. FR residues are present, e.g., in chimeric, Humanised, human, domain antibodies, diabodies, linear antibodies, and bispecific antibodies.

The term “heavy chain” when used in reference to an antibody refers to a polypeptide chain of about 50-70 kDa, wherein the amino-terminal portion includes a variable region of about 120 to 130 or more amino acids and a carboxy-terminal portion that includes a constant region. The constant region can be one of five distinct types, referred to as alpha (α), delta (δ), epsilon (ε), gamma (γ) and mu (μ), based on the amino acid sequence of the heavy chain constant region. The distinct heavy chains differ in size: α, δ and γ contain approximately 450 amino acids, while μ and ε contain approximately 550 amino acids. When combined with a light chain, these distinct types of heavy chains give rise to five well known classes of antibodies, IgA, IgD, IgE, IgG and IgM, respectively, including four subclasses of IgG, namely IgG1, IgG2, IgG3 and IgG4. A heavy chain can be a human heavy chain.

The term “hinge region” refers herein to a flexible amino acid stretch in the central part of the heavy chains of the IgG and IgA immunoglobulin classes, which links these 2 chains by disulfide bonds. The hinge region is generally defined as stretching from Glu216 to Pro230 of human IgG1 (Burton, Mol Immunol, 22: 161-206, 1985). Hinge regions of other IgG isotypes may be aligned with the IgG1 sequence by placing the first and last cysteine residues forming inter-heavy chain S—S bonds in the same positions. The “CH2 domain” of a human IgG Fc portion (also referred to as “Cy2” domain) usually extends from about amino acid 231 to about amino acid 340. The CH2 domain is unique in that it is not closely paired with another domain. Rather, two N-linked branched carbohydrate chains are interposed between the two CH2 domains of an intact native IgG molecule. It has been speculated that the carbohydrate may provide a substitute for the domain-domain pairing and help stabilise the CH2 domain (Burton, Mol Immunol, 22: 161-206, 1985). The “CH3 domain” comprises the stretch of residues C-terminal to a CH2 domain in an Fc portion (i.e., from about amino acid residue 341 to about amino acid residue 447 of an IgG).

The term “host” as used herein refers to an animal, such as a mammal (e.g., a human).

The term “host cell” as used herein refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

A “humanised” antibody refers to a chimeric antibody that contains minimal sequence derived from non-human immunoglobulin. In one embodiment, a humanised antibody is a human immunoglobulin (recipient antibody) in which residues from a CDR of the recipient are replaced by residues from a CDR of a non-human species (donor antibody) such as mouse, rat, rabbit, or nonhuman primate having the desired specificity, affinity, and/or capacity. In some instances, some of the skeleton segment residues (called FR for framework) can be modified to preserve binding affinity, according to techniques known by a man skilled in the art (Jones et al., Nature, 321:522-525, 1986). In some embodiments, FR residues of the human immunoglobulin are replaced by corresponding non-human residues. In certain embodiments, a humanised antibody will comprise substantially all of at least one, and typically two, variable domains, in which all or substantially all of the CDRs correspond to those of a non-human antibody, and all or substantially all of the FRs correspond to those of a human antibody. A humanised antibody optionally may comprise at least a portion of an antibody constant region (Fc), typically that of a human immunoglobulin. A “humanised form” of an antibody, e.g., a non-human antibody, refers to an antibody that has undergone humanisation. The goal of humanisation is a reduction in the immunogenicity of a xenogenic antibody, such as a murine antibody, for introduction into a human, while maintaining the full antigen binding affinity and specificity of the antibody. For further details, see, e.g., Jones et al, Nature 321: 522-525 (1986); Riechmann et al., Nature 332:323-329 (1988); and Presta, Curr. Op. Struct. Biol. 2:593-596 (1992). See also, e.g., Vaswani and Hamilton, Ann. Allergy, Asthma & Immunol. 1:105-115 (1998); Harris, Biochem. Soc. Transactions 23:1035-1038 (1995); Hurle and Gross, Curr. Op. Biotech. 5:428-433 (1994); and U.S. Pat. Nos. 6,982,321 and 7,087,409.

As used herein, “identifying” as it refers to a subject that has a condition refers to the process of assessing a subject and determining that the subject has a condition, for example, suffers from cancer.

As used herein, the terms “immune checkpoint” or “immune checkpoint protein” refer to certain proteins made by some types of immune system cells, such as T cells, and some cancer cells. Such proteins regulate T cell function in the immune system. Notably, they help keep immune responses in check and can keep T cells from killing cancer cells. Said immune checkpoint proteins achieve this result by interacting with specific ligands which send a signal into the T cell and essentially switch off or inhibit T cell function. Inhibition of these proteins results in restoration of T cell function and an immune response to the cancer cells. Examples of checkpoint proteins include, but are not limited to CTLA-4, PDL1, PDL2, PD1, B7-H3, B7-H4, BTLA, HVEM, TIGIT, TIM3, GAL9, LAG3, VSIG4, KIR, 2B4 (belongs to the CD2 family of molecules and is expressed on all NK, γδ, and memory CD8+ (αβ) T cells), CD160 (also referred to as BY55), CGEN-15049, CHK1 and CHK2 kinases, IDO1, A2aR, and various B7 family ligands.

The term “increased”, as used herein, refers to the activity of a protein, e.g., VISTA, at least 1-fold (e.g. 1, 2, 3, 4, 5, 10, 20, 30, 40, 50, 60, 70, 80, 90, 100, 1000, 10,000-fold or more) greater than its reference value. “Increased”, as it refers to the activity of a protein, e.g., VISTA, of a subject, signifies also at least 5% greater (e.g., 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100%) than the activity of the protein in the reference sample or with respect to the reference value for said protein. The term “increased”, as used herein, also refers to the level of a biomarker, e.g., VISTA, of a subject at least 1-fold (e.g. 1, 2, 3, 4, 5, 10, 20, 30, 40, 50, 60, 70, 80, 90, 100, 1000, 10,000-fold or more) greater than its reference value. “Increased”, as it refers to the level of a biomarker, e.g., VISTA, of a subject, signifies also at least 5% greater (e.g., 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, 99%, or 100%) than the level in the reference sample or with respect to the reference value for said marker.

The term “inhibit,” or “block”, or a grammatical equivalent thereof, when used in the context of an antibody refers to an antibody that suppresses, restrains or decreases a biological activity of the antigen to which the antibody binds. The inhibitory effect of an antibody can be one which results in a measurable change in the antigen's biological activity. In particular instances, “inhibit,” or “block” refers to an antibody that prevents or stops a biological activity of the antigen to which the antibody binds. A blocking antibody includes an antibody that combines with an antigen without eliciting a reaction, but that blocks another protein from later combining or complexing with that antigen. The blocking effect of an antibody can be one which results in a measurable change in the antigen's biological activity. In some embodiments, an anti-VISTA antibody described herein blocks the ability of VISTA to bind VSIG3, which can result in inhibiting or blocking suppressive signals of VISTA. Certain anti-VISTA antibodies described herein inhibit or block suppressive signals of VISTA on VISTA-expressing cells, including by about 98% to about 100% as compared to the appropriate control (e.g., the control being cells not treated with the antibody being tested). In some embodiments, the anti-VISTA antibody described herein blocks the binding of the extracellular domain VISTA to VSIG3 and/or blocks the binding of a VISTA-expressing cell to a VSIG3-expressing cell. In some embodiments, an anti-VISTA antibody described herein blocks the ability of VISTA to bind PSGL-1, preferably at acidic pH (pH between 5.9 and 6.5), which can result in inhibiting or blocking suppressive signals of VISTA. Certain anti-VISTA antibodies described herein inhibit or block suppressive signals of VISTA on VISTA-expressing cells, including by about 98% to about 100% as compared to the appropriate control (e.g., the control being cells not treated with the antibody being tested). In some embodiments, the anti-VISTA antibody described herein blocks, preferably at acidic pH (pH between 5.9 and 6.5), the binding of the extracellular domain VISTA to PSGL-1 and/or blocks, preferably at acidic pH (pH between 5.9 and 6.5), the binding of a VISTA-expressing cell to a PSGL-1-expressing cell.

The term “immune infiltrate” or “tumour immune cells” refers to cells that infiltrate the microenvironment of a tumour, including, but not limited to, lymphocytes (e.g., T cells, B-cells, natural killer (NK) cells), dendritic cells, mast cells, and macrophages.

As used herein, the term “in combination” in the context of the administration of other therapies refers to the use of more than one therapy (e.g., an anti-VISTA antibody and an immune checkpoint inhibitor such as an anti-PD-1 antibody or an anti-PD-L1 antibody). The use of the term “in combination” does not restrict the order or the time in which therapies are administered to a subject (e.g., one therapy before, concurrent with, or after another therapy). A first therapy can be administered before (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks), concurrently, or after (e.g., 1 minute, 45 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks) the administration of a second therapy to a subject which had, has, or is susceptible to a VISTA-mediated disease, disorder or condition. Any additional therapy can be administered in any order or time with the other additional therapies (e.g., an anti-VISTA antibody and an immune checkpoint inhibitor such as an anti-PD-1 antibody or an anti-PD-L1 antibody). In some embodiments, the antibodies can be administered in combination with one or more therapies (e.g., therapies that are not the antibodies that are currently administered to prevent, treat, manage, and/or ameliorate a VISTA-mediated disease, disorder or condition). Non-limiting examples of therapies that can be administered in combination with an antibody include an antagonist to a co-inhibitory molecule, an agonist to a co-stimulatory molecule, a chemotherapeutic agent, radiation, analgesic agents, anaesthetic agents, antibiotics, or immunomodulatory agents or any other agent listed in the U.S. Pharmacopoeia and/or Physician's Desk Reference.

An “isolated” antibody is one which has been separated from a component of its natural environment. In some embodiments, an antibody is purified to greater than 95% or 99% purity as determined by, for example, electrophoresis (e.g., SDS-PAGE, isoelectric focusing (IEF), capillary electrophoresis) or chromatography (e.g., ion exchange or reverse phase HPLC). For review of methods for assessment of antibody purity, see, e.g., Flatman et al., J. Chromatogr. B 848:79-87 (2007).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

The term “K_{D}” used herein means a dissociation constant of a specific antibody-antigen interaction and is used as an indicator for measuring the affinity of an antibody for an antigen. Lower K_{D} means higher affinity of an antibody for an antigen.

As intended herein, the “level” of a biomarker, e.g., VISTA, consists of a quantitative value of the biomarker in a sample, e.g., in a sample collected from a cancer-suffering patient. In some embodiments, the quantitative value does not consist of an absolute value that is actually measured, but rather consists of a final value resulting from taking into consideration of a signal to noise ratio occurring with the assay format used, and/or taking into consideration of calibration reference values that are used to increase reproducibility of the measures of the level of a cancer marker, from assay-to-assay. In some embodiments, the “level” of a biomarker, e.g., VISTA, is expressed as arbitrary units, since what is important is that the same kind of arbitrary units are compared (i) from assay-to-assay, or (ii) from one cancer-suffering patient to others, or (iii) from assays performed at distinct time periods for the same patient, or (iv) between the biomarker level measured in a patient's sample and a predetermined reference value (which may also be termed a “cut-off” value herein).

The term “light chain” when used in reference to an antibody refers to a polypeptide chain of about 25 kDa, wherein the amino-terminal portion includes a variable region of about 100 to about 110 or more amino acids and a carboxy-terminal portion that includes a constant region. The approximate length of a light chain is 211 to 217 amino acids. There are two distinct types, referred to as kappa (κ) of lambda (A) based on the amino acid sequence of the constant domains. Light chain amino acid sequences are well known in the art. A light chain can be a human light chain.

As used herein, the term “monoclonal antibody” designates an antibody arising from a nearly homogeneous antibody population, wherein population comprises identical antibodies except for a few possible naturally-occurring mutations which can be found in minimal proportions. A monoclonal antibody arises from the growth of a single cell clone, such as a hybridoma, and is characterised by heavy chains of one class and subclass, and light chains of one type. As used herein, a monoclonal antibody shows specific binding to a single antigenic site (i.e., single epitope) when the antibody is presented to it. The monoclonal antibody can be produced by various methods that are well known in the corresponding technical area.

The term “native” when used in connection with biological materials such as nucleic acid molecules, polypeptides, host cells, and the like, refers to those which are found in nature and not manipulated by a human being.

As described herein, the term “PEGylation” means a processing method for increasing the retention time of an antibody in blood by introducing polyethylene glycol to the aforementioned monoclonal antibody or an antigen-binding fragment thereof. Specifically, according to PEGylation of polymer nanoparticles with polyethylene glycol, hydrophilicity on a nanoparticle surface is enhanced, and, accordingly, fast degradation in living body can be prevented due to so-called stealth effect which prevents recognition by immune activity including macrophage in a human body to cause phagocytosis and digestion of pathogens, waste products, and foreign materials introduced from an outside. As such, the retention time of an antibody in blood can be increased by PEGylation. The PEGylation employed in the present disclosure can be carried out by a method by which an amide group is formed based on a bond between the carboxyl group of hyaluronic acid and the amine group of polyethylene glycol, but it is not limited thereto, and the PEGylation can be carried out by various methods. At that time, as for the polyethylene glycol to be used, polyethylene glycol having molecular weight of 100 to 1,000 and a linear or branched structure is preferably used, although it is not particularly limited thereto.

As used herein, the “percentage identity” or “% identity” between two sequences of nucleic acids or amino acids refers to the percentage of identical nucleotides or amino acid residues between the two sequences to be compared, obtained after optimal alignment, this percentage being purely statistical and the differences between the two sequences being distributed randomly along their length. The comparison of two nucleic acid or amino acid sequences is traditionally carried out by comparing the sequences after having optimally aligned them, said comparison being able to be conducted by segment or by using an “alignment window”. Optimal alignment of the sequences for comparison can be carried out, in addition to comparison by hand, by means of methods known by a man skilled in the art.

For the amino acid sequence exhibiting at least 70%, at least 75%, at least 80%, at least 85%, at least 90%, at least 91%, at least 92%, at least 93%, at least 94%, at least 95%, at least 96%, at least 97%, at least 98%, or at least 99% identity with a reference amino acid sequence, preferred examples include those containing the reference sequence, certain modifications, notably a deletion, addition or substitution of at least one amino acid, truncation or extension. In the case of substitution of one or more consecutive or non-consecutive amino acids, substitutions are preferred in which the substituted amino acids are replaced by “equivalent” amino acids. Here, the expression “equivalent amino acids” is meant to indicate any amino acids likely to be substituted for one of the structural amino acids without however modifying the biological activities of the corresponding antibodies and of those specific examples defined below. Equivalent amino acids can be determined either on their structural homology with the amino acids for which they are substituted or on the results of comparative tests of biological activity between the various antibodies likely to be generated.

As a non-limiting example, Table 1 below summarises the possible substitutions likely to be carried out without resulting in a significant modification of the biological activity of the corresponding modified antigen binding protein; inverse substitutions are naturally possible under the same conditions.

TABLE 1

| Original residue | Substitution(s) |
|---|---|
| Ala (A) | Val, Gly, Pro |
| Arg (R) | Lys, His |
| Asn (N) | Gln |
| Asp (D) | Glu |
| Cys (C) | Ser |
| Gln (Q) | Asn |
| Glu (E) | Asp |
| Gly (G) | Ala |
| His (H) | Arg |
| Ile (I) | Leu |
| Leu (L) | Ile, Val, Met |
| Lys (K) | Arg |
| Met (M) | Leu |
| Phe (F) | Tyr |
| Pro (P) | Ala |
| Ser (S) | Thr, Cys |
| Thr (T) | Ser |
| Trp (W) | Tyr |
| Tyr (Y) | Phe, Trp |
| Val (V) | Leu, Ala |

The term “pharmaceutically acceptable” as used herein means being approved by a regulatory agency of the Federal or a state government, or listed in the U.S. Pharmacopeia, European Pharmacopeia or other generally recognised Pharmacopeia for use in animals, and more particularly in humans. More specifically, when referring to a carrier, the expression “pharmaceutically acceptable” means that the carrier(s) is compatible with the other ingredient(s) of the composition and is not deleterious to the recipient thereof. Accordingly, as used herein, the expression “pharmaceutically acceptable carrier” refers to a carrier or a diluent which does not inhibit the biological activity and characteristics of a compound for administration without stimulating a living organism. The type of carrier can be selected based upon the intended route of administration. The amount of each carriers used may vary within ranges conventional in the art. As a pharmaceutically acceptable carrier in the composition which is prepared as a liquid solution, physiological saline, sterilised water, buffered saline, albumin injection solution, dextrose solution, maltodextrin solution, glycerol, and a mixture of one or more of them can be used as a sterilised carrier suitable for a living organism. If necessary, common additives like anti-oxidant, buffer solution, and bacteriostat may be added. Furthermore, by additionally adding a diluent, a dispersant, a surfactant, a binder, or a lubricant, the composition can be prepared as a formulation for injection like aqueous solution, suspension, and emulsion, a pill, a capsule, a granule, or a tablet.

As used herein, the term “polyclonal antibody” refers to an antibody which was produced among or in the presence of one or more other, non-identical antibodies. In general, polyclonal antibodies are produced from a B-lymphocyte in the presence of several other B-lymphocytes producing non-identical antibodies. Usually, polyclonal antibodies are obtained directly from an immunised animal.

As used herein, the term “polynucleotide,” “nucleotide,” nucleic acid” “nucleic acid molecule” and other similar terms are used interchangeable and include DNA, RNA, mRNA and the like.

The term “radiation,” when used in the therapeutic context refers to a type of treatment that uses a beam of intense energy to kill target cells (e.g., cancer cells). Radiation therapy includes the use of X-rays, protons or other forms of energy that are administered through an external beam. Radiation therapy also includes radiation treatment that is placed into a patient's body (e.g., brachytherapy) whereby a small container of radioactive material is implanted directly into or near a tumour.

The term “reference value”, as used herein, refers to the expression level of a biomarker under consideration (e.g., VISTA) in a reference sample. A “reference sample”, as used herein, means a sample obtained from subjects, preferably two or more subjects, known to be free of the disease or, alternatively, from the general population. The suitable reference expression levels of biomarker can be determined by measuring the expression levels of said biomarker in several suitable subjects, and such reference levels can be adjusted to specific subject populations. The reference value or reference level can be an absolute value; a relative value; a value that has an upper or a lower limit; a range of values; an average value; a median value, a mean value, or a value as compared to a particular control or baseline value. A reference value can be based on an individual sample value such as, for example, a value obtained from a sample from the subject being tested, but at an earlier point in time. The reference value can be based on a large number of samples, such as from population of subjects of the chronological age matched group, or based on a pool of samples including or excluding the sample to be tested.

As used herein, the term “side effects” encompasses unwanted and adverse effects of a therapy (e.g., a prophylactic or therapeutic agent). Unwanted effects are not necessarily adverse. An adverse effect from a therapy (e.g., a prophylactic or therapeutic agent) might be harmful or uncomfortable or risky. Examples of side effects include, diarrhoea, cough, gastroenteritis, wheezing, nausea, vomiting, anorexia, abdominal cramping, fever, pain, loss of body weight, dehydration, alopecia, dyspnoea, insomnia, dizziness, mucositis, nerve and muscle effects, fatigue, dry mouth, and loss of appetite, rashes or swellings at the site of administration, flu-like symptoms such as fever, chills and fatigue, digestive tract problems and allergic reactions. Additional undesired effects experienced by patients are numerous and known in the art. Many are described in the Physician's Desk Reference (67th ed., 2013).

The terms “stability” and “stable” as used herein in the context of a liquid formulation comprising an antibody (including antibody fragment thereof) that specifically binds to an antigen of interest (e.g., VISTA) refer to the resistance of the antibody (including antibody fragment thereof) in the formulation to aggregation, degradation or fragmentation under given manufacture, preparation, transportation and storage conditions. The “stable” formulations of the disclosure retain biological activity under given manufacture, preparation, transportation and storage conditions. The stability of said antibody (including antibody fragment thereof) can be assessed by degrees of aggregation, degradation or fragmentation, as measured by HPSEC, reverse phase chromatography, static light scattering (SLS), Dynamic Light Scattering (DLS), Fourier Transform Infrared Spectroscopy (FTIR), circular dichroism (CD), urea unfolding techniques, intrinsic tryptophan fluorescence, differential scanning calorimetry, and/or ANS binding techniques, compared to a reference formulation. For example, a reference formulation may be a reference standard frozen at −70° C. consisting of 20 mg/ml of an antibody (including antibody fragment thereof) (for example, but not limited to, an antibody comprising a heavy chain sequence of SEQ ID NO:21, a light chain sequence of SEQ ID NO: 22) in 25 mM histidine, pH 6.5 that contains 150 mM NaCl, and 0.3% polysorbate 80, which reference formulation regularly gives a single monomer peak (e.g., ≥97% area) by HPSEC. The overall stability of a formulation comprising an antibody (including antibody fragment thereof) can be assessed by various immunological assays including, for example, ELISA and radioimmunoassay using isolated antigen molecules.

A “subject” which may be subjected to the methodology described herein may be any of mammalian animals including human, dog, cat, cattle, goat, pig, swine, sheep and monkey. A human subject can be known as a patient. In one embodiment, “subject” or “subject in need” refers to a mammal that is suffering from cancer or is suspected of suffering from cancer or has been diagnosed with cancer. As used herein, a “cancer-suffering subject” refers to a mammal that is suffering from cancer or has been diagnosed with cancer. A “control subject” refers to a mammal that is not suffering from cancer, and is not suspected of suffering from cancer.

As used herein “substantially all” refers to refers to at least about 60%, at least about 70%, at least about 75%, at least about 80%, at least about 85%, at least about 90%, at least about 95%, at least about 98%, at least about 99%, or about 100%.

As used herein, the term “therapeutic agent” refers to any agent that can be used in treating, preventing or alleviating a disease, disorder or condition, including in the treatment, prevention or alleviation of one or more symptoms of a VISTA-mediated disease, disorder, or condition and/or a symptom related thereto. In some embodiments, a therapeutic agent refers to an anti-VISTA antibody provided herein. In some embodiments, a therapeutic agent refers to an agent other than an anti-VISTA antibody provided herein. In some embodiments, a therapeutic agent is an agent which is known to be useful for, or has been or is currently being used for the treatment, prevention or alleviation of one or more symptoms of a VISTA-mediated disease, disorder, condition, and/or a symptom related thereto.

The combination of therapies (e.g., use of therapeutic agents) can be more effective than the additive effects of any two or more single therapies. For example, a synergistic effect of a combination of therapeutic agents permits the use of lower dosages of one or more of the agents and/or less frequent administration of the agents to a subject with a VISTA-mediated disease, disorder or condition and/or a symptom related thereto. The ability to utilise lower dosages of therapeutic therapies and/or to administer the therapies less frequently reduces the toxicity associated with the administration of the therapies to a subject without reducing the efficacy of the therapies in the prevention, treatment or alleviation of one or more symptoms of a VISTA-mediated disease, disorder or condition and/or a symptom related thereto. In addition, a synergistic effect can result in improved efficacy of therapies in the prevention, treatment or alleviation of one or more symptoms of a VISTA-mediated disease, disorder or condition and/or a symptom related thereto. Finally, synergistic effect of a combination of therapies (e.g., therapeutic agents) may avoid or reduce adverse or unwanted side effects associated with the use of any single therapy.

The term “therapeutically effective amount” as used herein refers to the amount of a therapeutic agent (e.g., an anti-VISTA antibody or any other therapeutic agent, including as described herein, including, for example, an immune checkpoint inhibitor, such as e.g., an anti-PD-1 antibody or an anti-PD-L1 antibody) that is sufficient to reduce and/or ameliorate the severity and/or duration of a given disease, disorder or condition and/or a symptom related thereto. A therapeutically effective amount of a therapeutic agent can be an amount necessary for the reduction or amelioration of the advancement or progression of a given disease, disorder or condition, reduction or amelioration of the recurrence, development or onset of a given disease, disorder or condition and/or to improve or enhance the prophylactic or therapeutic effect of another therapy (e.g., a therapy other than the administration of an anti-VISTA antibody, including as described herein).

As used herein, the term “therapy” refers to any protocol, method and/or agent that can be used in the prevention, management, treatment and/or amelioration of a VISTA-mediated disease, disorder or condition. In some embodiments, the terms “therapies” and “therapy” refer to a biological therapy, supportive therapy, and/or other therapies useful in the treatment, prevention and/or amelioration of a VISTA-mediated disease, disorder or condition known to one of skill in the art such as medical personnel.

As used herein, “treating” a disease in a subject or “treating” a subject having a disease refers to subjecting the subject to a pharmaceutical treatment, e.g., the administration of a drug, such that the extent of the disease is decreased or prevented. For example, treating results in the reduction of at least one sign or symptom of the disease or condition. Treatment includes (but is not limited to) administration of a composition, such as a pharmaceutical composition, and may be performed either prophylactically, or subsequent to the initiation of a pathologic event. Treatment can require administration of an agent and/or treatment more than once. In some embodiments, such terms refer to the reduction or amelioration of the progression, severity, and/or duration of a disease, that is responsive to immune modulation, such modulation resulting from increasing T cell activation.

The term “tumour microenvironment” refers to the cellular environment in which a tumour exists. A tumour microenvironment can include surrounding blood vessels, immune cells, fibroblasts, bone marrow-derived inflammatory cells, lymphocytes, signalling molecules and the extracellular matrix.

The term “variable domain” or “variable region” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable domains differ extensively in sequence between different antibodies. The variability in sequence is concentrated in the CDRs while the less variable portions in the variable domain are referred to as framework regions (FR). Each variable region comprises three CDRs which are connected to four FR. The CDRs of the light and heavy chains are primarily responsible for the interaction of the antibody with antigen. Although not directly involved in antigen binding, the FR determines the folding of the molecules and thus the amount of CDR that is presented on the surface of the variable region for interaction with the antigen. In some embodiments, the variable region is a human variable region.

The “variable region” or “variable domain” of an antibody refers to the amino-terminal domains of the heavy or light chain of the antibody. The variable domain of the heavy chain may be referred to as “V_{H}.” The variable domain of the light chain may be referred to as “V_{L}.” These domains are generally the most variable parts of an antibody and contain the antigen-binding sites.

The term “variant” when used in relation to VISTA or to an anti-VISTA antibody refers to a peptide or polypeptide comprising one or more (such as, for example, about 1 to about 25, about 1 to about 20, about 1 to about 15, about 1 to about 10, or about 1 to about 5) amino acid sequence substitutions, deletions, and/or additions as compared to a native or unmodified sequence. For example, a VISTA variant may result from one or more (such as, for example, about 1 to about 25, about 1 to about 20, about 1 to about 15, about 1 to about 10, or about 1 to about 5) changes to an amino acid sequence of native VISTA. Also by way of example, a variant of an anti-anti-VISTA antibody may result from one or more (such as, for example, about 1 to about 25, about 1 to about 20, about 1 to about 15, about 1 to about 10, or about 1 to about 5) changes to an amino acid sequence of a native or previously unmodified anti-anti-VISTA antibody. Preferably, a variant of an anti-VISTA antibody may result from one change to an amino acid sequence of a native or previously unmodified anti-anti-VISTA antibody. In some embodiments, the VISTA variant or anti-VISTA antibody variant at least retains VISTA or anti-VISTA antibody functional activity, respectively. In some embodiments, an anti-VISTA antibody variant does not undergo deamidation in the CDRs. In some embodiments, an anti-VISTA antibody variant binds VISTA and/or is antagonistic to VISTA activity. In some embodiments, an anti-VISTA antibody variant does not undergo deamidation in the CDRs, binds VISTA and/or is antagonistic to VISTA activity. Variants may be naturally occurring, such as allelic or splice variants, or may be artificially constructed. In some embodiments, the variant is encoded by a single nucleotide polymorphism (SNP) variant of a nucleic acid molecule that encodes VISTA or anti-VISTA antibody VH or VL regions or subregions. Polypeptide variants may be prepared from the corresponding nucleic acid molecules encoding the variants.

The term “vector” refers to a substance that is used to introduce a nucleic acid molecule into a host cell. In particular, a “vector,” as used herein, is a nucleic acid molecule capable of propagating another nucleic acid molecule to which it is linked. One example of vector is a “plasmid”, which refers to a circular double stranded DNA loop into which additional DNA segments may be ligated. Another example of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. The term “vector” thus includes the vector as a self-replicating nucleic acid structure as well as the vector incorporated into the genome of a host cell into which it has been introduced. Vectors applicable for use include, for example, expression vectors, plasmids, phage vectors, viral vectors, episomes and artificial chromosomes, which can include selection sequences or markers operable for stable integration into a host cell's chromosome.

Certain vectors are capable of directing the expression of genes to which they are operatively linked. Such vectors are referred to herein as “recombinant expression vectors” (or simply, “expression vectors”). In general, expression vectors of utility in recombinant DNA techniques are in the form of plasmids. In the present specification, “plasmid” and “vector” may be used interchangeably as the plasmid is the most commonly used form of vector. However, the invention is intended to include such forms of expression vectors, such as bacterial plasmids, YACs, cosmids, retrovirus, EBV-derived episomes, and all the other vectors that the skilled man will know to be convenient for ensuring the expression of the heavy and/or light chains of the antibody of interest (e.g., an anti-VISTA antibody). The skilled man will realise that the polynucleotides encoding the heavy and the light chains can be cloned into different vectors or in the same vector.

The vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more nucleic acid molecules are to be co-expressed (e.g. both an antibody heavy and light chain), both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the nucleic acid molecule is expressed in a sufficient amount to produce the desired product (e.g. an anti-VISTA antibody provided herein), and it is further understood that expression levels can be optimised to obtain sufficient expression using methods well known in the art.

The term “VISTA” or “VISTA polypeptide” and similar terms refers to the polypeptide (“polypeptide,” “peptide” and “protein” are used interchangeably herein) encoded by the human Chromosome 10 Open Reading Frame 54 (VISTA) gene, which is also known in the art as B7-H5, platelet receptor Gi24, GI24, Stress Induced Secreted Protein1, SISP1, and PP2135, for example, comprising the amino acid sequence of:

| (SEQ ID NO: 1) | |
|---|---|
| 1 | mgvptaleag swrwgsllfa lflaaslgpv aafkvatpys lyvcpegqnv tltcrllgpv |
| 61 | dkghdvtfyk twyrssrgev qtcserrpir nltfqdlhlh hgghqaants hdlaqrhgle |
| 121 | sasdhhgnfs itmrnltlld sglycclvve irhhhsehrv hgamelqvqt gkdapsncvv |
| 181 | ypsssqdsen itaaalatga civgilclpl illlvykqrq aasnrraqel vrmdsniqgi |
| 241 | enpgfeaspp aqgipeakvr hplsyvaqrq psesgrhlls epstplsppg pgdvffpsld |
| 301 | pvpdspnfev i |

and related polypeptides, including SNP variants thereof. The VISTA polypeptide has been shown to or is predicted to comprise several distinct regions within the amino acid sequence including: a signal sequence (residues 1-32; see Zhang et al., Protein Sci. 13:2819-2824 (2004)); an immunoglobulin domain—IgV-like (residues 33-162); and a transmembrane region (residues 195-215). The mature VISTA protein includes amino acid residues 33-311 of SEQ ID NO: 1. The extracellular domain of the VISTA protein includes amino acid residues 33-194 of SEQ ID NO: 1. Related polypeptides include allelic variants (e.g., SNP variants); splice variants; fragments; derivatives; substitution, deletion, and insertion variants; fusion polypeptides; and interspecies homologs, preferably, which retain VISTA activity and/or are sufficient to generate an anti-VISTA immune response. VISTA can exist in a native or denatured form. The VISTA polypeptides described herein may be isolated from a variety of sources, such as from human tissue types or from another source, or prepared by recombinant or synthetic methods. A “native sequence VISTA polypeptide” comprises a polypeptide having the same amino acid sequence as the corresponding VISTA polypeptide derived from nature. Such native sequence VISTA polypeptides can be isolated from nature or can be produced by recombinant or synthetic means. The term “native sequence VISTA polypeptide” specifically encompasses naturally-occurring truncated or secreted forms of the specific VISTA polypeptide (e.g., an extracellular domain sequence), naturally-occurring variant forms (e.g., alternatively spliced forms) and naturally-occurring allelic variants of the polypeptide.

A cDNA nucleic acid sequence encoding the VISTA polypeptide, for example, comprises:

| (SEQ ID NO: 2) | |
|---|---|
| 1 | atgggcgtcc ccacggccct ggaggccggc agctggcgct ggggatccct gctcttcgct |
| 61 | ctcttcctgg ctgcgtccct aggtccggtg gcagccttca aggtcgccac gccgtattcc |
| 121 | ctgtatgtct gtcccgaggg gcagaacgtc accctcacct gcaggctctt gggccctgtg |
| 181 | gacaaagggc acgatgtgac cttctacaag acgtggtacc gcagctcgag gggcgaggtg |
| 241 | cagacctgct cagagcgccg gcccatccgc aacctcacgt tccaggacct tcacctgcac |
| 301 | catggaggcc accaggctgc caacaccagc cacgacctgg ctcagcgcca cgggctggag |
| 361 | tcggcctccg accaccatgg caacttctcc atcaccatgc gcaacctgac cctgctggat |
| 421 | agcggcctct actgctgcct ggtggtggag atcaggcacc accactcgga gcacagggtc |
| 481 | catggtgcca tggagctgca ggtgcagaca ggcaaagatg caccatccaa ctgtgtggtg |
| 541 | tacccatcct cctcccagga tagtgaaaac atcacggctg cagccctggc tacgggtgcc |
| 601 | tgcatcgtag gaatcctctg cctccccctc atcctgctcc tggtctacaa gcaaaggcag |
| 661 | gcagcctcca accgccgtgc ccaggagctg gtgcggatgg acagcaacat tcaagggatt |
| 721 | gaaaaccccg gctttgaagc ctcaccacct gcccagggga tacccgaggc caaagtcagg |
| 781 | caccccctgt cctatgtggc ccagcggcag ccttctgagt ctgggcggca tctgctttcg |
| 841 | gagcccagca cccccctgtc tcctccaggc cccggagacg tcttcttccc atccctggac |
| 901 | cctgtccctg actctccaaa ctttgaggtc atctag |

VISTA is predominantly expressed on the myeloid cell population, particularly myeloid-derived suppressor cells (MDSCs), neutrophils, monocytes, macrophages, and dendritic cells. VISTA can also be expressed on regulatory T cells and CD4⁺ naïve T lymphocytes. As described herein, VISTA is an immunomodulator, that is a negative checkpoint regulator of immune responses (e.g., inhibits or suppresses immune responses). VISTA has been identified as a negative checkpoint regulator of T cell function and is known to suppress autoimmune responses in a variety of human and mouse models of autoimmunity. VISTA has in particular been shown to promote tumourigenesis, block T cell function, and modulate the activity of macrophages and immunosuppressive myeloid-derived suppressor cells (MDSCs). VISTA is upregulated on immunosuppressive tumour infiltrating leukocytes such as inhibitory regulatory T cells (Tregs) and MDSCs. The presence of VISTA in the tumour microenvironment hinders effective T cell responses and has been implicated in a number of human cancers including prostate, colon, skin, pancreatic, and lung (ElTanbouly et al. Clin Exp Immunol. 200(2):120-130 (2020); Mehta et al. Sci Rep. 10(1):1 5171 (2020); Yuan et al. Trends Immunol. 42(3): 209-227 (2021); Tagliamento et al. Immunotargets Ther. 10: 185-200 (2021); Thakkar et al. J Immunother Cancer. 10(2): e003382 (2022); WO 2015/097536; WO 2016/094837; WO 2017/181139; WO 2019/183040)

Orthologs to the VISTA polypeptide are also well known in the art. For example, the mouse ortholog to the VISTA polypeptide is V-region Immunoglobulin-containing Suppressor of T cell Activation (VISTA) (also known as PD-L3, PD-1H, PD-XL, Pro1412 and UNQ730), which shares approximately 70% sequence identity to the human polypeptide. Orthologs of VISTA can also be found in additional organisms including chimpanzee, cow, rat and zebrafish.

A “VISTA-expressing cell,” “a cell having expression of VISTA” or a grammatical equivalent thereof refers to a cell that expresses endogenous or transfected VISTA on the cell surface. VISTA expressing cells include VISTA-bearing tumour cells, regulatory T cells (e.g., CD4⁺ Foxp3⁺ regulatory T cells), myeloid-derived suppressor cells (e.g., CD11b⁺ or CD11b^{high} myeloid-derived suppressor cells) and/or suppressive dendritic cells (e.g., CD11b⁺ or CD11b^{high} dendritic cells). A cell expressing VISTA produces sufficient levels of VISTA on its surface, such that an anti-VISTA antibody can bind thereto and/or PSGL-1 or a cell expressing PSGL-1 can bind thereto. In some aspects, inhibition or blocking of such binding may have a therapeutic effect. A cell that “overexpresses” VISTA is one that has significantly higher levels of VISTA at the cell surface thereof, compared to a cell of the same tissue type that is known to express VISTA. Such overexpression may be caused by gene amplification or by increased transcription or translation. VISTA overexpression may be determined in a diagnostic or prognostic assay by evaluating increased levels of the VISTA protein present on the surface of a cell (e.g. via an immunohistochemistry assay; FACS analysis). Alternatively, or additionally, one may measure levels of VISTA-encoding nucleic acid or mRNA in the cell, e.g. via fluorescent in situ hybridisation; (FISH; see WO98/45479 published October, 1998), Southern blotting, Northern blotting, or polymerase chain reaction (PCR) techniques, such as real time quantitative PCR (RT-PCR). Aside from the above assays, various in vivo assays are available to the skilled practitioner. For example, one may expose cells within the body of the patient to an antibody which is optionally labelled with a detectable agent, and binding of the antibody to cells in the patient can be evaluated, e.g. by external scanning for radioactivity or by analysing a biopsy taken from a patient previously exposed to the antibody. A VISTA-expressing tumour cell includes, but is not limited to, acute myeloid leukaemia (AML) tumour cells.

A “VISTA-mediated disease,” “VISTA-mediated disorder” and “VISTA-mediated condition” are used interchangeably and refer to any disease, disorder or condition that is completely or partially caused by or is the result of VISTA. Such diseases, disorders or conditions include those caused by or otherwise associated with VISTA, including by or associated with VISTA-expressing cells (e.g., tumour cells, myeloid-derived suppressor cells (MDSC), suppressive dendritic cells (suppressive DC), and/or regulatory T cells (T-regs)). In some embodiments, VISTA is aberrantly (e.g., highly) expressed on the surface of a cell. In some embodiments, VISTA may be aberrantly upregulated on a particular cell type. In other embodiments, normal, aberrant or excessive cell signalling is caused by binding of VISTA to a VISTA receptor (e.g., PSGL-1, VSIG3, VSIG8, or LRIG1), which can bind or otherwise interact with VISTA. Preferably, a “VISTA-mediated disease” as used herein refers to a tumour (i.e., a “VISTA-mediated tumour”) whose proliferation is associated with the activity of VISTA. For example, expression of VISTA in cells present in the tumour microenvironment, e.g., MDSCs, may result in suppression of an immune response against the tumour. In a specific instance, expression of VISTA in cells present in the tumour microenvironment, e.g., MDSCs, may result in suppression of T cell immunity (CD4⁺ and CD8⁺ T cell immunity) and/or prevention of the expression of proinflammatory cytokines. Notably, proliferation of CD4⁺ and or CD8⁺ T cells may be inhibited. The expression of cytokines such as IFNγ, IL-2, or TNFα, may be prevented. In a specific aspect, these effects are mediated by VISTA expressed on cells present in the tumour microenvironment, e.g., MDSCs, interacting with receptors such as PSG-L1, VSIG3, VSIG8, or LRIG1, which are expressed on immune cells, e.g., T cells, or tumour cells.

### Anti-VISTA Antibodies

Immune checkpoints play crucial roles in maintaining self-tolerance and limiting immune-mediated tissue damage under physiologic conditions. VISTA is a type-I transmembrane protein belonging to the B7-related immunoglobulin superfamily which is highly expressed in the haematopoietic compartment. VISTA acts both as a ligand and a receptor and negatively regulates T-cell activation through inhibiting CD4⁺ and CD8⁺ T-cell proliferation and proinflammatory cytokines (e.g., IFNγ, TNFα, or IL-2) production.

A monoclonal antibody Ab3 capable of inhibiting VISTA immune suppression, thereby enhancing antitumour immune response, is described in WO 2016/094837. The CDRS of this antibody are represented by SEQ ID NOS:3-8, and the V_{H} and V_{L} by SEQ ID NO:9 and SEQ ID NO: 10, respectively. The complete heavy chain of Ab3 has the sequence represented by SEQ ID NO: 11 and the complete light chain of Ab3 has the sequence represented by SEQ ID NO: 12.

The complete sequence of the antibody Ab3 indicates that it contains 11 potential deamidation sites. Whereas all these sites are predicted to be valid deamidation sites, the present inventors have found that only one of them is actually subject to deamidation, i.e., the Asn residue at position in 55 in CDR2 of the heavy chain. Surprisingly, the replacement of this Asn by an Asp residue does not affect the binding of Ab3 to its target, in stark contrast to the teaching of the prior art wherein a similar mutation in a CDR led to a 400-fold decrease of the affinity for the corresponding CD52 antigen (Liu et al., 2022). Moreover, the mutated antibody retains the capacity of inhibiting VISTA immunoinhibitory activity, since it is capable of blocking the interaction between VISTA and each of its two binding partners, PSG-L1 and VSIG3, said interaction resulting in inhibition of T cell function. Accordingly, the mutated antibody inhibits tumour proliferation in vivo.

In a first aspect, the present disclosure provides a novel anti-VISTA antibody wherein the antibody comprises a substitution of an Asp for an Asn in the CDR2 of the heavy chain.

Anti-VISTA monoclonal antibodies as used herein include, but are not limited to, synthetic antibodies, recombinantly produced antibodies, multispecific antibodies (including bi-specific antibodies), human antibodies, humanised antibodies, camelised antibodies, chimeric antibodies, intrabodies, anti-idiotypic (anti-Id) antibodies, and functional fragments of any of the above. Anti-VISTA monoclonal antibodies can be of human or non-human origin. Examples of anti-VISTA antibodies of non-human origin include but are not limited to, those of mammalian origin (e.g., simians, rodents, goats, and rabbits). Because every structure of the human antibody originates from a human, there is only low probability of having an immune response compared to a conventional humanised antibody or mouse antibody, and thus it has an advantage that it does not cause any undesirable immune response when administered to a human. Therefore, it can be very advantageously used as an antibody for treatment. Accordingly, anti-VISTA monoclonal antibodies for therapeutic use in humans are preferably humanised or fully human. More preferably, they are humanised.

The antibody disclosed herein is an antibody with substantially the same affinity to the antigen as the antibody Ab3. The term “affinity” indicates a property of specifically recognising and binding to a specific antigen site, and, together with specificity of an antibody for an antigen, the high affinity is an important factor in an immune reaction. In the present case, affinity of the presently disclosed antibodies may be determined by competitive ELISA. Other than this method, various methods for measuring the affinity for an antigen may be employed, and the surface plasmon resonance technology is one example of those methods.

Within a range in which VISTA can be specifically recognised, the monoclonal antibody disclosed herein may include not only the sequence of anti-VISTA antibody of the present invention, which is described in the present specification, but also a biological equivalent thereof, wherein the biological equivalent displays improved binding affinity and/or other biological characteristics of an antibody. For example, to have further improvement of the binding affinity and/or other biological characteristics of an antibody, additional changes can be made on the amino acid sequence of an antibody. Included in those modifications are deletion, insertion, and/or substitution of the amino acid sequence of an antibody, for example. Those modifications of an amino acid are made based on relative similarity among side-chain substituents of an amino acid, for example, hydrophobicity, hydrophilicity, charge, size, or the like. Based on the analysis of the size, shape, and type of the side-chain substituents of an amino acid, it is found that all of arginine, lysine, and histidine are a residue with positive charge; alanine, glycine, and serine have a similar size; and phenylalanine, tryptophan, and tyrosine have a similar shape. Accordingly, it can be said based on those considerations that, biologically, arginine, lysine, and histidine; alanine, glycine, and serine; and phenylalanine, tryptophan, and tyrosine are functional equivalents.

In an embodiment, the anti-VISTA monoclonal antibodies described herein can be in the form of full-length antibodies, multiple chain or single chain antibodies, fragments of such antibodies that selectively bind to VISTA (including but not limited to Fab, Fab′, (Fab′)₂, Fv, and scFv), surrobodies (including surrogate light chain construct), single domain antibodies, humanised antibodies, camelised antibodies and the like. They also can be of, or derived from, any isotype, including, for example, IgA (e.g., Ig1l or lgA2), IgD, IgE, IgG (e.g., IgG1, IgG2, IgG3 or IgG4), or IgM. In some embodiments, the anti-VISTA antibody is an IgG (e.g., IgG1, IgG2, IgG3 or IgG4). In an embodiment, the antibody further comprises a human constant region. In a further embodiment, the human constant region is selected from the group consisting of IgG1, IgG2, IgG2, IgG3 and IgG4. In a still further specific embodiment, the human constant region is IgG1. Furthermore, the heavy chain constant region has gamma (γ), mu (μ), alpha (α), delta (δ) and epsilon (ε) types, and, as a subclass, it has gamma1 (γ1), gamma2 (γ2), gamma3 (γ3), gamma4 (γ4), alpha1 (α1) and alpha2 (α2). The light chain constant region has kappa (κ) and lambda (λ) types.

In the context of the present disclosure, antibodies comprising a human IgG1 constant region are particularly preferred. Not only do they bind to VISTA with the same affinity as the antibody Ab3, they are also capable of inhibiting the VISTA immunosuppressive effect. Surprisingly, this activity requires the effector functions of the antibodies, which had never been documented for the anti-VISTA antibody Ab3.

Preferably, the anti-VISTA antibody disclosed herein comprises a heavy chain of sequence SEQ ID NO:21 and a light chain of sequence SEQ ID NO:22.

This antibody is more stable and more homogeneous than the antibody Ab3, since it comprises an Asp at position 55 and is thus not subject to deamidation. In addition, this antibody has the same affinity as the antibody Ab3 and inhibits VISTA immune suppressive activity. This inhibition is, in particular, the result of the disruption of the interaction between VISTA and each of its binding partners, PSG-L1 and VSIG-3. In contrast, there is no indication that the antibody Ab3 interferes with these interactions. Moreover, the inhibition of VISTA immune suppression surprisingly requires the effector functions of the antibody disclosed herein.

Anti-VISTA antibodies include labelled antibodies, useful in diagnostic applications. The antibodies can be used diagnostically, for example, to detect expression of a target of interest in specific cells, tissues, or serum; or to monitor the development or progression of an immunologic response as part of a clinical testing procedure to, e.g., determine the efficacy of a given treatment regimen. Detection can be facilitated by coupling the antibody to a detectable substance or “label.” A label can be conjugated directly or indirectly to an anti-VISTA antibody of the disclosure. The label can itself be detectable (e.g., radioisotope labels, isotopic labels, or fluorescent labels) or, in the case of an enzymatic label, can catalyse chemical alteration of a substrate compound or composition which is detectable. Examples of detectable substances include various enzymes, prosthetic groups, fluorescent materials, luminescent materials, bioluminescent materials, radioactive materials, positron emitting metals using various positron emission tomographies, and nonradioactive paramagnetic metal ions. The detectable substance can be coupled or conjugated either directly to the antibody (or fragment thereof) or indirectly, through an intermediate (such as, for example, a linker known in the art) using techniques known in the art. Examples of enzymatic labels include luciferases (e.g., firefly luciferase and bacterial luciferase; U.S. Pat. No. 4,737,456), luciferin, 2,3-dihydrophthalazinediones, malate dehydrogenase, urease, peroxidase such as horseradish peroxidase (HRPO), alkaline phosphatase, β-galactosidase, acetylcholinesterase, glucoamylase, lysozyme, saccharide oxidases (e.g., glucose oxidase, galactose oxidase, and glucose-6-phosphate dehydrogenase), heterocyclic oxidases (such as uricase and xanthine oxidase), lactoperoxidase, microperoxidase, and the like. Examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride, dimethylamine-1-napthalenesulfonyl chloride, or phycoerythrin and the like; an example of a luminescent material includes luminol; examples of bioluminescent materials include luciferase, luciferin, and aequorin; examples of suitable isotopic materials include ¹³C, ¹⁵N, and deuterium; and examples of suitable radioactive material include ¹²⁵I, ¹³¹I, ¹¹¹In or ⁹⁹Tc.

### Bispecific Antibodies

In addition, the present disclosure provides a multi-specific antibody including the monoclonal anti-VISTA antibody disclosed herein or an antigen-binding fragment thereof.

The above multi-specific antibody in the present invention can preferably be a bi-specific antibody, but not limited thereto.

The multi-specific antibody according to the present invention preferably has the form in which the anti-VISTA antibody described herein is bound to an antibody having a binding property for an immunoeffector cell-specific target molecule, or a fragment thereof. The immunoeffector cell-specific target molecule is preferably an immune checkpoint, but it is not limited thereto. Examples of immunoeffector cell-specific target molecules include e.g., PD-1, PD-L1, CTLA-4, TIM-3, TIGIT, BTLA, KIR, A2aR, VSIG4, B7-H3, TCR/CD3, CD16 (FcγRIIIa) CD44, Cd56, CD69, CD64 (FcγRI), CD89 and CD11b/CD18 (CR3).

The multi-specific antibody is an antibody which can simultaneously recognise different multi (bi or higher) epitopes of the same antigen or two or more separate antigens, and the antibodies belonging to multi-specific antibody can be classified into scFv-based antibody, Fab-based antibody, IgG-based antibody, or the like. In case of a multi-specific, e.g., bi-specific, antibody, two signals can be simultaneously suppressed or amplified, and thus it can be more effective than a case in which one signal is suppressed/amplified. Compared to a case in which each signal is treated with a signal inhibitor for each, low-dose administration can be achieved and two signals can be suppressed/amplified at the same time in the same space.

Methods for producing a bi-specific antibody are widely known. Conventionally, recombination production of a bi-specific antibody is based on coexpression of a pair of heavy chain/light chain of two immunogloubulins under conditions at which two heavy chains have different specificity.

In case of a scFv-based bi-specific antibody, by combining VL and VH of different scFvs, a hybrid scFv-based is prepared in heterodimer form to give a diabody (Holliger et al., Proc. Natl. Acad. Sci. U.S.A., 90:6444, 1993), and, by connecting different scFvs to each other, tandem ScFv can be produced. By expressing CH1 and CL of Fab at the terminus of each scFv, a heterodimeric mini antibody can be produced (Muller et al., FEBS lett., 432:45, 1998). In addition, by substituting partial amino acids of CH3 domain as a homodimeric domain of Fc, a structural change into “knob into hole” form to have a heterodimer structure is made and those modified CH3 domains are expressed at the terminus of each different scFv, and thus a minibody in heterodimeric scFv form can be produced (Merchant et al., Nat. Biotechnol., 16:677, 1998).

In case of a Fab-based bi-specific antibody, according to combination of separate Fab′ for a specific antigen by utilising a disulfide bond or a mediator, the antibody can be produced in heterodimeric Fab form, and, by expressing scFv for a different antigen at the terminus of a heavy chain or a light chain of a specific Fab, the antigen valency of 2 can be obtained. In addition, by having a hinge region between Fab and scFv, the antigen valency of 4 can be obtained in homodimer form. In addition, a method of producing the followings is known in the pertinent art: a dual target bibody by which the antigen valency of 3 is obtained according to fusion of scFv for a different antigen at the light chain terminus and heavy chain terminus of Fab, a triple target bibody by which the antigen valency of 3 is obtained according to fusion of different scFvs to the light chain terminus and heavy chain terminus of Fab, and a triple target antibody F(ab′)₃ in simple form that is obtained by chemical fusion of three different Fabs.

In case of IgG-based bi-specific antibody, a method of producing bi-specific antibody by preparing hybrid hybridoma, so-called quadromas, based on re-hybridisation of mouse and rat hybridomas is known by Trion Pharma. In addition, a method of producing a bi-specific antibody in so-called “Holes and Knob” form, in which partial amino acids of the CH3 homodimeric domain of Fc in different heavy chains are modified while sharing the light chain part, is known (Merchant et al., Nat. Biotechnol., 16:677, 1998), and, other than the bi-specific antibody in heterodimer form, a method of producing (scFv)₄-IgG in homodimer form according to fusion of two different scFvs to the constant domain of the light chain and heavy chain of IgG instead of the variable domain, followed by expression, is known. Furthermore, it has been reported by ImClone Systems that, based on IMC-1C11 as a chimeric monoclonal antibody for human VEGFR-2, only a single variable domain for mouse platelet-derived growth factor receptor-α is fused to the amino terminus of the light chain of the antibody so as to produce a bi-specific antibody. Furthermore, an antibody having high antigen valency for CD20 has been reported by Rossi et al. based on so-called “dock and lock (DNL)” method using a dimerisation and docking domain (DDD) of protein kinase A (PKA) R subunit and an anchoring domain of PKA (Rossi et al., Proc. Natl. Acad. Sci. U.S.A., 103:6841, 2006).

### Antibody Derivatives

The anti-VISTA antibodies of the present invention can be further modified to contain additional non-proteinaceous moieties that are known in the art and readily available. In particular, included herein are anti-VISTA monoclonal antibodies which are derivatised, covalently modified, or conjugated to other molecules, for use in diagnostic and therapeutic applications. For example, but not by way of limitation, derivatised antibodies include antibodies that have been modified, e.g., by glycosylation, acetylation, pegylation, phosphorylation, derivatisation by known protecting/blocking groups, proteolytic cleavage, linkage to a cellular ligand or other protein, etc. Any of numerous chemical modifications can be carried out by known techniques, including, but not limited to, specific chemical cleavage, acetylation, formylation, metabolic synthesis of tunicamycin, etc. Additionally, the derivative can contain one or more non-classical amino acids.

In particular, the monoclonal antibody of the present invention or an antigen-binding fragment thereof may be subjected to derivatisation as described above, notably by e.g., glycosylation and/or PEGylation, in order to enhance the residence time in a living body to which the antibody is administered.

As for the glycosylation and/or PEGylation, various patterns of glycosylation and/or PEGylation can be modified by a method well known in the art, as long as the function of the antibody of the present invention is maintained, and included in the antibody of the present invention are a variant monoclonal antibody in which various patterns of glycosylation and/or PEGylation are modified, or an antigen-binding fragment thereof.

Preferably, the moieties suitable for derivatisation of the antibody are water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-l,3,6-trioxane, ethylene/maleic anhydride copolymer, polyaminoacids (either homopolymers or random copolymers), and dextran or poly(n-vinyl pyrrolidone) polyethylene glycol, propropylene glycol homopolymers, polypropylene oxide/ethylene oxide co-polymers, polyoxyethylated polyols (e.g., glycerol), polyvinyl alcohol, and mixtures thereof. The polymer may be of any molecular weight, and may be branched or unbranched. The number of polymers attached to the antibody may vary, and if more than one polymer are attached, they can be the same or different molecules. In general, the number and/or type of polymers used for derivatisation can be determined based on considerations including, but not limited to, the particular properties or functions of the antibody to be improved, whether the antibody derivative will be used in a therapy under defined conditions, etc.

In a specific example, the anti-VISTA antibodies of the present disclosure can be attached to Poly(ethyleneglycol) (PEG) moieties. In a specific embodiment, the antibody is an antibody fragment and the PEG moieties are attached through any available amino acid side-chain or terminal amino acid functional group located in the antibody fragment, for example any free amino, imino, thiol, hydroxyl or carboxyl group. Such amino acids can occur naturally in the antibody fragment or can be engineered into the fragment using recombinant DNA methods. See, for example U.S. Pat. No. 5,219,996. Multiple sites can be used to attach two or more PEG molecules. PEG moieties can be covalently linked through a thiol group of at least one cysteine residue located in the antibody fragment. Where a thiol group is used as the point of attachment, appropriately activated effector moieties, for example thiol selective derivatives such as maleimides and cysteine derivatives, can be used.

In a specific example, an anti-VISTA antibody conjugate is a modified Fab′ fragment which is PEGylated, i.e., has PEG (poly(ethyleneglycol)) covalently attached thereto, e.g., according to the method disclosed in EP0948544. See also Poly(ethyleneglycol) Chemistry, Biotechnical and Biomedical Applications, (J. Milton Harris (ed.), Plenum Press, New York, 1992); Poly(ethyleneglycol) Chemistry and Biological Applications, (J. Milton Harris and S. Zalipsky, eds., American Chemical Society, Washington D.C., 1997); and Bioconjugation Protein Coupling Techniques for the Biomedical Sciences, (M. Aslam and A. Dent, eds., Grove Publishers, New York, 1998); and Chapman, 2002, Advanced Drug Delivery Reviews 54:531-545. PEG can be attached to a cysteine in the hinge region. In one example, a PEG-modified Fab′ fragment has a maleimide group covalently linked to a single thiol group in a modified hinge region. A lysine residue can be covalently linked to the maleimide group and to each of the amine groups on the lysine residue can be attached a methoxypoly(ethyleneglycol) polymer having a molecular weight of approximately 20,000 Da. The total molecular weight of the PEG attached to the Fab′ fragment can therefore be approximately 40,000 Da.

In another embodiment, conjugates of an antibody and non-proteinaceous moiety that may be selectively heated by exposure to radiation are provided. In one embodiment, the non-proteinaceous moiety is a carbon nanotube (Kam et al, Proc. Natl. Acad. Sci. USA 102: 11600-11605 (2005)). The radiation may be of any wavelength, and includes, but is not limited to, wavelengths that do not harm ordinary cells, but which heat the non-proteinaceous moiety to a temperature at which cells proximal to the antibody-non-proteinaceous moiety are killed.

### Immunoconjugates

In another aspect, the present disclosure provides an immunoconjugate (interchangeably referred to as “antibody-drug conjugates,” or “ADCs”) comprising an anti-VISTA antibody as described herein, said antibody being conjugated to a cytotoxic agent.

Many cytotoxic agents have been isolated or synthesised and make it possible to inhibit the cells proliferation, or to destroy or reduce, if not definitively, at least significantly the tumour cells. However, the toxic activity of these agents is not limited to tumour cells, and the non-tumour cells are also affected and can be destroyed. More particularly, side effects are observed on rapidly renewing cells, such as haematopoietic cells or cells of the epithelium, in particular of the mucous membranes. In order to limit side effects on normal cells whilst retaining high cytotoxicity on tumour cells, immunoconjugates have been used for the local delivery of cytotoxic agents in the treatment of cancer (Lambert, J. (2005) Curr. Opinion in Pharmacology 5:543-549; Wu et al (2005) Nature Biotechnology 23(9): 1137-1146; Payne, G. (2003) i 3:207-212; Syrigos and Epenetos (1999) Anticancer Research 19:605-614; Niculescu-Duvaz and Springer (1997) Adv. Drug Deliv. Rev. 26:151-172; U.S. Pat. No. 4,975,278). Immunoconjugates allow for the targeted delivery of a drug moiety (i.e., the cytotoxic agent) to a tumour, and intracellular accumulation therein, where systemic administration of unconjugated drugs may result in unacceptable levels of toxicity to normal cells as well as the tumour cells sought to be eliminated (Baldwin et al, Lancet (Mar. 15, 1986) pp. 603-05; Thorpe (1985) “Antibody Carriers Of Cytotoxic Agents In Cancer Therapy: A Review,” in Monoclonal Antibodies '84: Biological And Clinical Applications (A. Pinchera et al., eds) pp. 475-506. Both polyclonal antibodies and monoclonal antibodies have been reported as useful in these strategies (Rowland et al., (1986) Cancer Immunol. Immunother. 21:183-87).

The cytotoxic agent used in the immunoconjugates disclosed herein may be, without limitation, a drug (i.e., “antibody-drug conjugate”), a toxin (i.e., “immunotoxin” or “antibody-toxin conjugate”), a radioisotope (i.e., “radioimmunoconjugate” or “antibody-radioisotope conjugate”), etc.

Preferably, the immunoconjugate is a binding protein linked to at least a drug or a medicament. Such an immunoconjugate is usually referred to as an antibody-drug conjugate (or “ADC”) when the binding protein is an antibody, or an antigen binding fragment thereof.

In a first embodiment, such drugs can be described regarding their mode of action. As non-limitative examples, it can be mentioned alkylating agents such as nitrogen mustard, alkyl-sulfonates, nitrosourea, oxazophorins, aziridines or imine-ethylenes, anti-metabolites, anti-tumour antibiotics, mitotic inhibitors, chromatin function inhibitors, anti-angiogenesis agents, anti-ooestrogens, anti-androgens, chelating agents, iron absorption stimulant, cyclooxygenase inhibitors, phosphodiesterase inhibitors, DNA inhibitors, DNA synthesis inhibitors, apoptosis stimulants, thymidylate inhibitors, T cell inhibitors, interferon agonists, ribonucleoside triphosphate reductase inhibitors, aromatase inhibitors, ooestrogen receptor antagonists, tyrosine kinase inhibitors, cell cycle inhibitors, taxane, tubulin inhibitors, angiogenesis inhibitors, macrophage stimulants, neurokinin receptor antagonists, cannabinoid receptor agonists, dopamine receptor agonists, granulocytes stimulating factor agonists, erythropoietin receptor agonists, somatostatin receptor agonists, LHRH agonists, calcium sensitizers, VEGF receptor antagonists, interleukin receptor antagonists, osteoclast inhibitors, radical formation stimulants, endothelin receptor antagonists, vinca alkaloid, anti-hormone or immunomodulators or any other new drug that fulfils the activity criteria of a cytotoxic or a toxin.

Such drugs are, for example, cited in VIDAL 2010, on the page devoted to the compounds attached to the cancerology and haematology column “Cytotoxics”, these cytotoxic compounds cited with reference to this document are cited here as preferred cytotoxic agents.

More particularly, without limitation, the following drugs are preferred according to the invention: mechlorethamine, chlorambucol, melphalen, chlorhydrate, pipobromen, prednimustin, disodic-phosphate, estramustine, cyclophosphamide, altretamine, trofosfamide, sulfofosfamide, ifosfamide, thiotepa, triethylenamine, altetramine, carmustine, streptozocin, fotemustin, lomustine, busulfan, treosulfan, improsulfan, dacarbazine, cis-platinum, oxaliplatin, lobaplatin, heptaplatin, miriplatin hydrate, carboplatin, methotrexate, pemetrexed, 5-fluoruracil, floxuridine, 5-fluorodeoxyuridine, capecitabine, cytarabine, fludarabine, cytosine arabinoside, 6-mercaptopurine (6-MP), nelarabine, 6-thioguanine (6-TG), chlorodesoxyadenosine, 5-azacytidine, gemcitabine, cladribine, deoxycoformycin, tegafur, pentostatin, doxorubicin, daunorubicin, idarubicin, valrubicin, mitoxantrone, dactinomycin, mithramycin, plicamycin, mitomycin C, bleomycin, procarbazine, paclitaxel, docetaxel, vinblastine, vincristine, vindesine, vinorelbine, topotecan, irinotecan, etoposide, valrubicin, amrubicin hydrochloride, pirarubicin, elliptinium acetate, zorubicin, epirubicin, idarubicin and teniposide, razoxin, marimastat, batimastat, prinomastat, tanomastat, ilomastat, CGS-27023A, halofuginon, COL-3, neovastat, thalidomide, CDC 501, DMXAA, L-651582, squalamine, endostatin, SU5416, SU6668, interferon-alpha, EMD121974, interleukin-12, IM862, angiostatin, tamoxifen, toremifene, raloxifene, droloxifene, iodoxyfene, anastrozole, letrozole, exemestane, flutamide, nilutamide, sprironolactone, cyproterone acetate, finasteride, cimitidine, bortezomid, velcade, bicalutamide, cyproterone, flutamide, fulvestran, exemestane, dasatinib, erlotinib, gefitinib, imatinib, lapatinib, nilotinib, sorafenib, sunitinib, retinoid, rexinoid, methoxsalene, methylaminolevulinate, aldesleukine, OCT-43, denileukin diflitox, interleukin-2, tasonermine, lentinan, sizofilan, roquinimex, pidotimod, thymopentine, poly I:C, procodazol, Tic BCG, Corynebacterium parvum, NOV-002, ukrain, levamisole, 1311-chTNT, H-101, celmoleukin, interferon alfa2a, interferon alfa2b, interferon gamma1a, interleukin-2, mobenakin, Rexin-G, teceleukin, aclarubicin, actinomycin, arglabin, asparaginase, carzinophilin, chromomycin, daunomycin, leucovorin, masoprocol, neocarzinostatin, peplomycin, sarkomycin, solamargine, trabectedin, streptozocin, testosterone, kunecatechins, sinecatechins, alitretinoin, belotecan hydrocholoride, calusterone, dromostanolone, elliptinium acetate, ethinyl estradiol, etoposide, fluoxymesterone, formestane, fosfetrol, goserelin acetate, hexyl aminolevulinate, histrelin, hydroxyprogesterone, ixabepilone, leuprolide, medroxyprogesterone acetate, megesterol acetate, methylprednisolone, methyltestosterone, miltefosine, mitobronitol, nadrolone phenylpropionate, norethindrone acetate, prednisolone, prednisone, temsirrolimus, testolactone, triamconolone, triptorelin, vapreotide acetate, zinostatin stimalamer, amsacrine, arsenic trioxide, bisantrene hydrochloride, chlorambucil, chlortrianisene, cis-diamminedichloroplatinium, cyclophosphamide, diethylstilbestrol, hexamethylmelamine, hydroxyurea, lenalidomide, lonidamine, mechlorethanamine, mitotane, nedaplatin, nimustine hydrochloride, pamidronate, pipobroman, porfimer sodium, ranimustine, razoxane, semustine, sobuzoxane, mesylate, triethylenemelamine, zoledronic acid, camostat mesylate, fadrozole HCl, nafoxidine, aminoglutethimide, carmofur, clofarabine, cytosine arabinoside, decitabine, doxifluridine, enocitabine, fludarabne phosphate, fluorouracil, ftorafur, uracil mustard, abarelix, bexarotene, raltiterxed, tamibarotene, temozolomide, vorinostat, megastrol, clodronate disodium, levamisole, ferumoxytol, iron isomaltoside, celecoxib, ibudilast, bendamustine, altretamine, mitolactol, temsirolimus, pralatrexate, TS-1, decitabine, bicalutamide, flutamide, letrozole, clodronate disodium, degarelix, toremifene citrate, histamine dihydrochloride, DW-166HC, nitracrine, decitabine, irinoteacn hydrochloride, amsacrine, romidepsin, tretinoin, cabazitaxel, vandetanib, lenalidomide, ibandronic acid, miltefosine, vitespen, mifamurtide, nadroparin, granisetron, ondansetron, tropisetron, alizapride, ramosetron, dolasetron mesilate, fosaprepitant dimeglumine, nabilone, aprepitant, dronabinol, TY-10721, lisuride hydrogen maleate, epiceram, defibrotide, dabigatran etexilate, filgrastim, pegfilgrastim, reditux, epoetin, molgramostim, oprelvekin, sipuleucel-T, M-Vax, acetyl L-carnitine, donepezil hydrochloride, 5-aminolevulinic acid, methyl aminolevulinate, cetrorelix acetate, icodextrin, leuprorelin, metbylphenidate, octreotide, amlexanox, plerixafor, menatetrenone, anethole dithiolethione, doxercalciferol, cinacalcet hydrochloride, alefacept, romiplostim, thymoglobulin, thymalfasin, ubenimex, imiquimod, everolimus, sirolimus, H-101, lasofoxifene, trilostane, incadronate, gangliosides, pegaptanib octasodium, vertoporfin, minodronic acid, zoledronic acid, gallium nitrate, alendronate sodium, etidronate disodium, disodium pamidronate, dutasteride, sodium stibogluconate, armodafinil, dexrazoxane, amifostine, WF-10, temoporfin, darbepoetin alfa, ancestim, sargramostim, palifermin, R-744, nepidermin, oprelvekin, denileukin diftitox, crisantaspase, buserelin, deslorelin, lanreotide, octreotide, pilocarpine, bosentan, calicheamicin, maytansinoids and ciclonicate.

For more detail, the person skilled in the art may refer to the manual edited by the “Association Française des Enseignants de Chimie Thérapeutique” and entitled “Traité de chimie thérapeutique, vol. 6, Médicaments antitumouraux et perspectives dans le traitement des cancers, edition TEC & DOC, 2003”.

Alternatively, the immunoconjugate may comprise a binding protein linked to at least a radioisotope. Such an immunoconjugate is usually referred to as an antibody-radioisotope conjugate (or “ARC”) when the binding protein is an antibody, or an antigen binding fragment thereof.

For selective destruction of the tumour, the antibody may comprise a highly radioactive atom. A variety of radioactive isotopes are available for the production of ARC such as, without limitation, At²¹¹, C¹³, N¹⁵, O¹⁷, Fl¹⁹, I¹²³, I¹³¹, I¹²⁵, In¹¹¹, Y⁹⁰, Re¹⁸⁶, Re¹⁸⁸, Sm¹⁵³, tc⁹⁹m, Bi²¹², P³², Pb²¹², radioactive isotopes of Lu, gadolinium, manganese or iron.

Any methods or processes known by the person skilled in the art can be used to incorporate such radioisotope in the ARC (see, for example “Monoclonal Antibodies in Immunoscintigraphy”, Chatal, CRC Press 1989). As non-limitative examples, Tc⁹⁹m or I¹²³, Re¹⁸⁶, Re¹⁸ and In¹¹¹ can be attached via a cysteine residue. Y⁹⁰ can be attached via a lysine residue. I¹²³ can be attached using the IODOGEN method (Fraker et al (1978) Biochem. Biophys. Res. Commun. 80: 49-57).

Several examples can be mentioned to illustrate the knowledge of the person skilled in the art in the field of ARC such as Zevalin® which is an ARC composed of an anti-CD20 monoclonal antibody and In¹¹¹ or Y⁹⁰ radioisotope bound by a thiourea linker-chelator (Wiseman et at (2000) Eur. Jour. Nucl. Med. 27(7):766-77; Wiseman et al (2002) Blood 99(12):4336-42; Witzig et at (2002) J. Clin. Oncol. 20(10):2453-63; Witzig et al (2002) J. Clin. Oncol. 20(15): 3262-69); or Mylotarg® which is composed of an anti-CD33 antibody linked to calicheamicin, (U.S. Pat. Nos. 4,970,198; 5,079,233; 5,585,089; 5,606,040; 5,693,762; 5,739,116; 5,767,285; 5,773,001). More recently, it can also be mentioned the ADC referred as Adcetris (corresponding to the Brentuximab vedotin) which has been recently accepted by the FDA in the treatment of Hodgkin's lymphoma (Nature, 476: 380-381, 25 Aug. 2011).

In yet another embodiment of the disclosure, the immunoconjugate may comprise a binding protein linked to a toxin. Such an immunoconjugate is usually referred to as an antibody-toxin conjugate (or “ATC”) when the binding protein is an antibody, or an antigen binding fragment thereof.

Toxins are effective and specific poisons produced by living organisms. They usually consist of an amino acid chain whose molecular weight may vary between a couple of hundred (peptides) and one hundred thousand daltons (proteins). They may also be low-molecular organic compounds. Toxins are produced by numerous organisms, e.g., bacteria, fungi, algae and plants. Many of them are extremely poisonous, with a toxicity that is several orders of magnitude greater than the nerve agents.

Toxins used in ATC can include, without limitation, all kind of toxins which may exert their cytotoxic effects by mechanisms including tubulin binding, DNA binding, or topoisomerase inhibition.

Enzymatically active toxins and fragments thereof that can be used include diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

Small molecule toxins, such as dolastatins, auristatins, a trichothecene, and CC1065, and the derivatives of these toxins that have toxin activity, are also contemplated herein. Dolastatins and auristatins have been shown to interfere with microtubule dynamics, GTP hydrolysis, and nuclear and cellular division and have anticancer and antifungal activity.

The immunoconjugates described herein may further comprise a linker.

“Linker”, “Linker Unit”, or “link” means a chemical moiety comprising a covalent bond or a chain of atoms that covalently attaches a binding protein to at least one cytotoxic agent.

Linkers may be made using a variety of bifunctional protein coupling agents such as N-succinimidyl-3-(2-pyridyldithio) propionate (SPDP), succinimidyl-4-(N-maleimidomethyl)cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Carbon-14-labelled 1-isothiocyanatobenzyl-3-methyldiethylene triaminepentaacetic acid (MX-DTPA) is an exemplary chelating agent for conjugation of cytotoxic agents to the addressing system. Other cross-linker reagents may be BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, Ill., U.S.A).

The linker may be a “non-cleavable” or “cleavable” linker.

Preferably, the linker is a “cleavable linker” facilitating release of the cytotoxic agent in the cell. For example, an acid-labile linker, a peptidase-sensitive linker, a photolabile linker, a dimethyl linker or a disulfide-containing linker may be used. The linker is preferably cleaved under intracellular conditions, such that cleavage of the linker releases the cytotoxic agent from the binding protein in the intracellular environment.

For example, in some embodiments, the linker may be cleaved by a cleaving agent that is present in the intracellular environment (e.g., within a lysosome or endosome or caveolea). The linker can be, for example, a peptidyl linker that is cleaved by an intracellular peptidase or protease enzyme, including, but not limited to, a lysosomal or endosomal protease. Typically, the peptidyl linker is at least two amino acids long or at least three amino acids long. Cleaving agents can include cathepsins B and D and plasmin, all of which are known to hydrolyse dipeptide drug derivatives resulting in the release of active drug inside target cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker). In specific embodiments, the peptidyl linker cleavable by an intracellular protease is a Val-Cit linker or a Phe-Lys linker. One advantage of using intracellular proteolytic release of the cytotoxic agent is that the agent is typically attenuated when conjugated and the serum stabilities of the conjugates are typically high.

In other embodiments, the cleavable linker is pH-sensitive, i.e., sensitive to hydrolysis at certain pH values. Typically, the pH-sensitive linker is hydrolysable under acidic conditions. For example, an acid-labile linker that is hydrolysable in the lysosome (e.g., a hydrazone, semicarbazone, thiosemicarbazone, cis-aconitic amide, orthoester, acetal, ketal, or the like) can be used. Such linkers are relatively stable under neutral pH conditions, such as those in the blood, but are unstable at below pH 5.5 or 5.0, the approximate pH of the lysosome. In certain embodiments, the hydrolysable linker is a thioether linker (such as, e.g., a thioether attached to the therapeutic agent via an acylhydrazone bond.

In yet other embodiments, the linker may be cleaved under reducing conditions (e.g., a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate), and SMPT (N-succinimidyl-oxycarbonyl-alpha-methyl-alpha-(2-pyridyl-dithio) toluene).

Non-cleavable linkers by contrast have no obvious drug release mechanism. Immunoconjugates comprising such non-cleavable linkers rely on the complete lysosomal proteolytic degradation of the antibody that releases the cytotoxic agent after internalisation.

As an example of an immunoconjugate comprising a non-cleavable linker, the immunoconjugate trastuzumab-emtansine (TDM1) can be mentioned, which combines trastuzumab with a linked chemotherapeutic agent, maytansin (Cancer Research 2008; 68: (22). Nov. 15, 2008).

In a preferred embodiment, the immunoconjugate disclosed herein may be prepared by any method known by the person skilled in the art such as, without limitation, i) reaction of a nucleophilic group of the antigen binding protein with a bivalent linker reagent followed by reaction with the cytotoxic agent or ii) reaction of a nucleophilic group of a cytotoxic agent with a bivalent linker reagent followed by reaction with the nucleophilic group of the antigen binding protein.

Nucleophilic groups on antigen binding protein include, without limitation, N-terminal amine groups, side chain amine groups, e.g. lysine, side chain thiol groups, and sugar hydroxyl or amino groups when the antigen binding protein is glycosylated. Amine, thiol, and hydroxyl groups are nucleophilic and capable of reacting to form covalent bonds with electrophilic groups on linker moieties and linker reagents including, without limitation, active esters such as NHS esters, HOBt esters, haloformates, and acid halides; alkyl and benzyl halides such as haloacetamides; aldehydes, ketones, carboxyl, and maleimide groups. The antigen binding protein may have reducible interchain disulfides, i.e. cysteine bridges. The antigen binding proteins may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into the antigen binding protein through any reaction known by the person skilled in the art. As non-limitative example, reactive thiol groups may be introduced into the antigen binding protein by introducing one or more cysteine residues.

Immunoconjugates may also be produced by modification of the antigen binding protein to introduce electrophilic moieties, which can react with nucleophilic substituents on the linker reagent or cytotoxic agent. The sugars of glycosylated antigen binding protein may be oxidised to form aldehyde or ketone groups which may react with the amine group of linker reagents or cytotoxic agent. The resulting imine Schiff base groups may form a stable linkage, or may be reduced to form stable amine linkages. In one embodiment, reaction of the carbohydrate portion of a glycosylated antigen binding protein with either galactose oxidase or sodium meta-periodate may yield carbonyl (aldehyde and ketone) groups in the protein that can react with appropriate groups on the drug. In another embodiment, proteins containing N-terminal serine or threonine residues can react with sodium meta-periodate, resulting in production of an aldehyde in place of the first amino acid.

### Chimeric Antigen Receptors

The present disclosure further provides a CAR (chimeric antigen receptor) protein including i) the antibody of the present invention; ii) a transmembrane domain, and; iii) an intracellular signalling domain characterised by causing T cell activation according to binding of the antibody of above i) to an antigen.

In the present disclosure, the CAR protein is characterised in that it is constituted by the monoclonal antibody of the present invention, a publicly known transmembrane domain, and an intracellular signalling domain

As described herein, the term “CAR (chimeric antigen receptor)” refers to a non-natural receptor capable of providing specificity for a specific antigen to an immunoeffector cell. In general, the CAR indicates a receptor that is used for providing the specificity of a monoclonal antibody to T cells. The CAR is generally constituted with an extracellular domain, a transmembrane domain and an intracellular domain. The extracellular domain includes an antigen recognition region, and, in the present description, the antigen recognition site is a VISTA-specific antibody. The VISTA-specific antibody is as described above, and the antibody used in CAR is preferably in the form of an antibody fragment. It is more preferably in the form of Fab or scFv, but not limited thereto.

Furthermore, the transmembrane domain of CAR has the form in which it is connected to the extracellular domain, and it may be originated from either natural or synthetic form. When it is originated from natural form, it may be originated from a membrane-bound or transmembrane protein, and it can be a part originated from transmembrane domains of various proteins like alpha, beta or zeta chain of T cell receptor, CD28, CD3 epsilon, CD45, CD4, CD5, CDS, CD9, CD16, CD22, CD33, CD37, CD64, CD80, CD86, CD134, CD137, CD154 or CD8. Sequences of those transmembrane domains can be obtained from documents that are well known in the art, in which the transmembrane domain of a transmembrane protein is described well, but it is not limited thereto.

The CAR described herein is the part of intracellular CAR domain, and it is connected to the transmembrane domain. The intracellular domain of the present invention may include an intracellular signalling domain, which is characterised by having a property of causing T cell activation, preferably T cell proliferation, upon binding of an antigen to the antigen recognition site of CAR. The intracellular signalling domain is not particularly limited in terms of the type thereof as long as it can cause the T cell activation upon binding of an antigen to the antigen recognition site of CAR present outside a cell, and various kinds of an intracellular signalling domain can be used. Examples thereof include immunoreceptor tyrosine based activation motif (ITAM), and the ITAM may include those originating from CD3 zeta (ξ), FcR gamma, FcR beta, CD3 gamma, CD3 delta, CD3 epsilon, CDS, CD22, CD79a, CD79b, CD66d or FcεRIγ, but not limited thereto.

Furthermore, it is preferable that the intracellular domain of the CAR of the present disclosure additionally comprises a costimulatory domain with the intracellular signalling domain, but not limited thereto. The costimulatory domain is a part which is comprised in the CAR described herein and plays a role of transferring a signal to T cells in addition to the signal from the intracellular signalling domain, and it indicates the intracellular part of CAR including the intracellular domain of a costimulatory molecule.

The costimulatory molecule means, as a cell surface molecule, a molecule required for having a sufficient reaction of lymphocytes for an antigen, and examples thereof include CD27, CD28, 4-1BB, OX40, CD30, CD40, PD-1, ICOS, LFA-1 (lymphocyte function-associated antigen-1), CD2, CD7, LIGHT, NKG2C, and B7-H3, but not limited thereto. The costimulatory domain can be an intracellular part of a molecule that is selected from the group consisting of those costimulatory molecules and a combination thereof.

Furthermore, selectively, a short oligopeptide or polypeptide linker may link the intracellular domain and transmembrane domain of CAR. Although this linker may be included in the CAR of the present invention, it is not particularly limited in terms of the linker length as long as it can induce the T cell activation via the intracellular domain binding of an antigen to an extracellular antibody.

### Nucleic Acids and Expression Systems

The present disclosure encompasses polynucleotides encoding immunoglobulin light and heavy chain genes for antibodies, notably anti-VISTA antibodies, vectors comprising such nucleic acids, and host cells capable of producing the antibodies of the disclosure. Also provided herein are polynucleotides that hybridise under high stringency, intermediate or lower stringency hybridisation conditions, e.g., as defined supra, to polynucleotides that encode an antibody or modified antibody provided herein.

In a first aspect, the present disclosure relates to one or more polynucleotides encoding an antibody, notably an antibody capable of binding specifically to VISTA, or a fragment thereof, as described above. The present disclosure notably provides a polynucleotide encoding the heavy chain and/or the light chain of the anti-VISTA antibody disclosed herein. More specifically, in certain embodiments, nucleic acid molecules provided herein comprise or consist of a nucleic acid sequence encoding the heavy chain variable region and light chain variable region disclosed herein, or any combination thereof (e.g., as a nucleotide sequence encoding an antibody provided herein, such as e.g., a full-length antibody, heavy and/or light chain of an antibody, or a single chain antibody provided herein).

For example, the polynucleotide encodes three heavy-chain CDRs of the anti-VISTA antibody described herein. For example, the polynucleotide encodes three light-chain CDRs of the anti-VISTA antibody described herein. For example, the polynucleotide encodes three heavy-chain CDRs and three light-chain CDRs of the anti-VISTA antibody described herein. Another example provides a couple of polynucleotides, wherein the first polynucleotide encodes three heavy-chain CDRs of the anti-VISTA antibody described herein; and the second polynucleotide encodes three light-chain CDRs of the same anti-VISTA antibody described herein.

In another instance, the polynucleotide encodes the heavy-chain variable region of the anti-VISTA antibody described herein. For instance, the polynucleotide encodes the light-chain variable region of the anti-VISTA antibody described herein. For instance, the polynucleotide encodes the heavy-chain variable region and the light-chain variable region of the anti-VISTA antibody described herein. Another instance provides a couple of polynucleotides, wherein the first polynucleotide encodes the heavy-chain variable region of the anti-VISTA antibody described herein; and the second polynucleotide encodes the light-chain variable region of the same anti-VISTA antibody described herein.

In an embodiment, the polynucleotide encodes the heavy-chain of the anti-VISTA antibody described herein. In an embodiment, the polynucleotide encodes the light-chain of the anti-VISTA antibody described herein. In an embodiment, the polynucleotide encodes the heavy-chain and the light-chain of the anti-VISTA antibody described herein. Another embodiment provides a couple of polynucleotides, wherein the first polynucleotide encodes the heavy-chain of the anti-VISTA antibody described herein; and the second polynucleotide encodes the light-chain of the same anti-VISTA antibody described herein.

In an embodiment, the polynucleotide encodes the heavy chain of the anti-VISTA antibody described above is provided. Preferably, the heavy chain comprises three heavy-chain CDRs of sequence SEQ ID NOS: 13-15. More preferably, the heavy chain comprises a heavy chain comprising the variable region of sequence SEQ ID NO:19. Even more preferably, the heavy chain has the sequence represented by SEQ ID NO:21.

In another embodiment, the polynucleotide encodes the light chain of an anti-VISTA antibody described above. Preferably, said light chain comprises three light-chain CDRs of sequence SEQ ID NOS: 16-18. More preferably, said light chain comprises a light chain comprising the variable region of sequence SEQ ID NO:20. Even more preferably, the light chain has the sequence represented by SEQ ID NO:222.

Due to the codon degeneracy or in consideration of a codon preferred in an organism in which the light chain and heavy chain of human antibody or a fragment thereof is to be expressed, the polynucleotide encoding the light chain and heavy chain of the monoclonal antibody of the present invention or an antigen-binding fragment thereof can have various variations in the coding region within a range in which the amino acid sequence of the light chain and heavy chain of an antibody expressed from the coding region is not changed, and, even in a region other than the coding region, various changes or modifications can be made within a range in which the gene expression is not affected by them. The skilled person will easily understand that those variant genes also fall within the scope of the present invention. Namely, as long as a protein having the equivalent activity is encoded by the polynucleotide of the present invention, one or more nucleic acid bases can be changed by substitution, deletion, insertion, or a combination thereof, and those also fall within the scope of the present invention. Sequence of the polynucleotide may be either a single chain or a double chain, and it may be either a DNA molecule or an RNA (mRNA) molecule.

According to the invention, a variety of expression systems may be used to express the antibody of the invention. In one aspect, such expression systems represent vehicles by which the coding sequences of interest may be produced and subsequently purified, but also represent cells which may, when transiently transfected with the appropriate nucleotide coding sequences, express an IgG antibody in situ.

The disclosure provides vectors comprising the polynucleotides described above. In one embodiment, the vector contains a polynucleotide encoding a heavy chain of the anti-VISTA antibody of interest. In another embodiment, the polynucleotide encodes the light chain of the anti-VISTA antibody of interest. In another embodiment, the polynucleotide encodes the heavy chain and the light chain of the anti-VISTA antibody of interest. In yet another embodiment, a couple of polynucleotides are provided, wherein the first polynucleotide encodes the heavy chain of the anti-VISTA antibody of interest, and the second polynucleotide encodes the light chain of the same anti-VISTA antibody of interest.

The disclosure also provides vectors comprising polynucleotide molecules encoding fusion proteins, modified antibodies, antibody fragments, and probes thereof.

In order to express the heavy and/or light chain of the anti-VISTA antibody of interest, the polynucleotides encoding said heavy and/or light chains are inserted into expression vectors such that the genes are operatively linked to transcriptional and translational sequences. In a preferred embodiment, these polynucleotides are cloned into two vectors.

“Operably linked” sequences include both expression control sequences that are contiguous with the gene of interest and expression control sequences that act in trans or at a distance to control the gene of interest. The term “expression control sequence” as used herein refers to polynucleotide sequences which are necessary to affect the expression and processing of coding sequences to which they are ligated. Expression control sequences include appropriate transcription initiation, termination, promoter and enhancer sequences; efficient RNA processing signals such as splicing and polyadenylation signals; sequences that stabilise cytoplasmic mRNA; sequences that enhance translation efficiency (i.e., Kozak consensus sequence); sequences that enhance protein stability; and when desired, sequences that enhance protein secretion. The nature of such control sequences differs depending upon the host organism; in prokaryotes, such control sequences generally include promoter, ribosomal binding site, and transcription termination sequence; in eukaryotes, generally, such control sequences include promoters and transcription termination sequence. The term “control sequences” is intended to include, at a minimum, all components whose presence is essential for expression and processing, and can also include additional components whose presence is advantageous, for example, leader sequences and fusion partner sequences.

Polynucleotides of the invention and vectors comprising these molecules can be used for the transformation of a suitable host cell. The term “host cell”, as used herein, is intended to refer to a cell into which a recombinant expression vector has been introduced in order to express the anti-VISTA antibody of interest. It should be understood that such terms are intended to refer not only to the particular subject cell but also to the progeny of such a cell. Because certain modifications may occur in succeeding generations due to either mutation or environmental influences, such progeny may not, in fact, be identical to the parent cell, but are still included within the scope of the term “host cell” as used herein.

Transformation can be performed by any known method for introducing polynucleotides into a cell host. Such methods are well known of the man skilled in the art and include dextran-mediated transformation, calcium phosphate precipitation, polybrene-mediated transfection, protoplast fusion, electroporation, encapsulation of the polynucleotide into liposomes, biolistic injection and direct microinjection of DNA into nuclei.

The host cell may be co-transfected with one or more expression vectors. For example, a host cell can be transfected with a vector encoding both the heavy chain and the light chain of the anti-VISTA antibody of interest, as described above. Alternatively, the host cell can be transformed with a first vector encoding the heavy chain of the anti-VISTA antibody of interest, and with a second vector encoding the light chain of said antibody. Mammalian cells are commonly used for the expression of a recombinant therapeutic immunoglobulins, especially for the expression of whole recombinant antibodies. For example, mammalian cells such as HEK293 or CHO cells, in conjunction with a vector, containing the expression signal such as one carrying the major intermediate early gene promoter element from human cytomegalovirus, are an effective system for expressing the humanised anti-VISTA antibody of the invention (Foecking et al., 1986, Gene 45:101; Cockett et al., 1990, Bio/Technology 8: 2).

In addition, a host cell may be chosen which modulates the expression of the inserted sequences, or modifies and processes the gene product in the specific fashion desired. Such modifications (e.g., glycosylation) and processing of protein products may be important for the function of the protein. Different host cells have features and specific mechanisms for the post-translational processing and modification of proteins and gene products. Appropriate cell lines or host systems are chosen to ensure the correct modification and processing of the expressed antibody of interest. Hence, eukaryotic host cells which possess the cellular machinery for proper processing of the primary transcript, glycosylation of the gene product may be used. Such mammalian host cells include, but are not limited to, CHO, COS, HEK293, NS/0, BHK, Y2/0, 3T3 or myeloma cells (all these cell lines are available from public depositories such as the Collection Nationale des Cultures de Microorganismes, Paris, France, or the American Type Culture Collection, Manassas, VA, U.S.A.).

For long-term, high-yield production of recombinant proteins, stable expression is preferred. In one embodiment of the invention, cell lines which stably express the antibody may be engineered. Rather than using expression vectors which contain viral origins of replication, host cells are transformed with DNA under the control of the appropriate expression regulatory elements, including promoters, enhancers, transcription terminators, polyadenylation sites, and other appropriate sequences known to the person skilled in art, and a selectable marker. Following the introduction of the foreign DNA, engineered cells may be allowed to grow for one to two days in an enriched media, and then are moved to a selective media. The selectable marker on the recombinant plasmid confers resistance to the selection and allows cells to stably integrate the plasmid into a chromosome and be expanded into a cell line. Other methods for constructing stable cell lines are known in the art. In particular, methods for site-specific integration have been developed. According to these methods, the transformed DNA under the control of the appropriate expression regulatory elements, including promoters, enhancers, transcription terminators, polyadenylation sites, and other appropriate sequences is integrated in the host cell genome at a specific target site which has previously been cleaved (Moele et al., Proc. Natl. Acad. Sci. U.S.A., 104(9): 3055-3060; U.S. Pat. Nos. 5,792,632; 5,830,729; 6,238,924; WO 2009/054985; WO 03/025183; WO 2004/067753).

A number of selection systems may be used according to the invention, including but not limited to the Herpes simplex virus thymidine kinase (Wigler et al., Cell 11:223, 1977), hypoxanthine-guanine phosphoribosyltransferase (Szybalska et al., Proc Natl Acad Sci USA 48: 202, 1992), glutamate synthase selection in the presence of methionine sulfoximide (Adv Drug Del Rev, 58: 671, 2006, and website or literature of Lonza Group Ltd.) and adenine phosphoribosyltransferase (Lowy et al., Cell 22: 817, 1980) genes in tk, hgprt or aprt cells, respectively. Also, antimetabolite resistance can be used as the basis of selection for the following genes: dhfr, which confers resistance to methotrexate (Wigler et al., Proc Natl Acad Sci USA 77: 357, 1980); gpt, which confers resistance to mycophenolic acid (Mulligan et al., Proc Natl Acad Sci USA 78: 2072, 1981); neo, which confers resistance to the aminoglycoside, G-418 (Wu et al., Biotherapy 3: 87, 1991); and hygro, which confers resistance to hygromycin (Santerre et al., Gene 30: 147, 1984). Methods known in the art of recombinant DNA technology may be routinely applied to select the desired recombinant clone, and such methods are described, for example, in Ausubel et al., eds., Current Protocols in Molecular Biology, John Wiley & Sons (1993). The expression levels of an antibody can be increased by vector amplification. When a marker in the vector system expressing an antibody is amplifiable, an increase in the level of inhibitor present in the culture will increase the number of copies of the marker gene. Since the amplified region is associated with the gene encoding the IgG antibody of the invention, production of said antibody will also increase (Crouse et al., Mol Cell Biol 3: 257, 1983). Alternative methods of expressing the gene of the invention exist and are known to the person of skills in the art. For example, a modified zinc finger protein can be engineered that is capable of binding the expression regulatory elements upstream of the gene of the invention; expression of the said engineered zinc finger protein (ZFN) in the host cell of the invention leads to increases in protein production (see e.g. Reik et al., Biotechnol. Bioeng., 97(5): 1180-1189, 2006). Moreover, ZFN can stimulate the integration of a DNA into a predetermined genomic location, resulting in high-efficiency site-specific gene addition (Moehle et al, Proc Natl Acad Sci USA, 104: 3055, 2007).

The anti-VISTA antibody of interest may be prepared by growing a culture of the transformed host cells under culture conditions necessary to express the desired antibody. The resulting expressed antibody may then be purified from the culture medium or cell extracts. Soluble forms of the anti-VISTA antibody of interest can be recovered from the culture supernatant. It may then be purified by any method known in the art for purification of an immunoglobulin molecule, for example, by chromatography (e.g., ion exchange, affinity, particularly by Protein A affinity for Fc, and so on), centrifugation, differential solubility or by any other standard technique for the purification of proteins. Suitable methods of purification will be apparent to a person of ordinary skills in the art.

Another aspect of the invention thus relates to a method for the production of an antibody (e.g., an anti-VISTA antibody) described herein, said method comprising the steps of:
a) growing the above-described host cell in a culture medium under suitable culture conditions; and
b) recovering the antibody (e.g., an anti-VISTA antibody), from the culture medium or from said cultured cells.

The antibody obtained by culturing the transformant can be used in a non-purified state. Impurities can be removed by additional various commons methods like centrifuge or ultrafiltration, and the resultant may be subjected to dialysis, salt precipitation, chromatography or the like, in which the method may be used either singly or in combination thereof. Among them, affinity chromatography is most widely used, including ion exchange chromatography, size exclusion chromatography, hydrophobic interaction chromatography, hydroxyapatite chromatography, and the like.

### Pharmaceutical Compositions

In another aspect, the present disclosure provides compositions comprising an anti-VISTA antibody or an antigen-binding fragment thereof, such as e.g., any of the anti-VISTA antibodies described herein, or a conjugate thereof, i.e., an immunoconjugate comprising one of the anti-VISTA antibodies described herein.

These compositions are particularly useful for e.g., stimulating an immune response in a subject. The antibody of the present invention which specifically binds to VISTA induces T cell activation by binding to VISTA protein, which inhibits T cell activation, and thus the antibody can stimulate an immune response.

The compositions described herein are also useful for treating cancer. A protective anti-tumour immunity can be established by administration of such compositions comprising the anti-VISTA antibody, antigen-binding fragments thereof, or conjugates thereof, which are disclosed herein.

Optionally, the compositions can comprise one or more additional therapeutic agents, such as the immune checkpoint inhibitors described below. The compositions will usually be supplied as part of a sterile, pharmaceutical composition that will normally include a pharmaceutically acceptable carrier and/or excipient. In another aspect, the invention thus provides a pharmaceutical composition comprising the anti-VISTA antibody or antigen-binding fragment thereof or conjugates thereof, and a pharmaceutically acceptable carrier and/or an excipient.

This composition can be in any suitable form (depending upon the desired method of administering it to a patient). The compositions utilised in the methods described herein can be administered, for example, intravitreally (e.g., by intravitreal injection), by eye drop, intramuscularly, intravenously, intradermally, percutaneously, intraarterially, intraperitoneally, intralesionally, intracranially, intraarticularly, intraprostatically, intrapleurally, intratracheally, intrathecally, intranasally, intravaginally, intrarectally, topically, intratumourally, peritoneally, subcutaneously, subconjunctivally, intravesicularly, mucosally, intrapericardially, intraumbilically, intraocularly, intraorbitally, orally, topically, transdermally, by inhalation, by injection, by implantation, by infusion, by continuous infusion, by localised perfusion bathing target cells directly, by catheter, by lavage, in cremes, or in lipid compositions. The compositions utilised in the methods described herein can also be administered systemically or locally. The method of administration can vary depending on various factors (e.g., the compound or composition being administered and the severity of the condition, disease, or disorder being treated). The most suitable route for administration in any given case will depend on the particular antibody, the subject, and the nature and severity of the disease and the physical condition of the subject. For example, the anti-VISTA antibody, an antigen-binding fragment thereof, or its conjugate can be formulated as an aqueous solution and administered by subcutaneous injection. Preferably, the anti-VISTA is formulated as an aqueous solution and administered by infusion.

Pharmaceutical compositions can be conveniently presented in unit dose forms containing a predetermined amount of an anti-VISTA, an antigen-binding fragment thereof, or a conjugate thereof per dose. Such a unit can contain for example but without limitation 5 mg to 5 g, for example 10 mg to 1 g, or 20 to 50 mg. Pharmaceutically acceptable carriers for use in the disclosure can take a wide variety of forms depending, e.g., on the condition to be treated or route of administration.

Pharmaceutical compositions of the disclosure can be prepared for storage as lyophilised formulations or aqueous solutions by mixing the antibody having the desired degree of purity with optional pharmaceutically-acceptable carriers, excipients or stabilisers typically employed in the art (all of which are referred to herein as “carriers”), i.e.g., buffering agents, stabilising agents, preservatives, isotonifiers, non-ionic detergents, antioxidants, and other miscellaneous additives. See, Remington's Pharmaceutical Sciences, 16th edition (Osol, ed. 1980). Such additives must be nontoxic to the recipients at the dosages and concentrations employed. Preferably, the composition disclosed herein is a liquid composition. More preferably, the liquid composition of the disclosure is an aqueous composition. Still more preferably, the liquid composition of the disclosure is an aqueous composition wherein the aqueous carrier is distilled water.

Advantageously, the composition of the disclosure is sterile.

Advantageously, the composition of the disclosure is homogeneous.

Advantageously, the composition of the disclosure is isotonic.

The disclosure encompasses stable liquid compositions comprising a single antibody of interest, for example, an antibody that specifically binds to VISTA as described herein. The disclosure also encompasses stable liquid compositions comprising two or more antibodies of interest (including antibody fragments thereof), for example, antibodies that specifically bind to an ICOS polypeptide(s). In one embodiment, a composition of the disclosure comprises at least about 1 mg/ml, at least about 5 mg/ml, at least about 10 mg/ml, at least about 20 mg/ml, at least about 30 mg/ml, at least about 40 mg/ml, at least about 50 mg/ml, at least about 60 mg/ml, at least about 70 mg/ml, at least about 80 mg/ml, at least about 90 mg/ml, at least about 100 mg/ml, at least about 110 mg/ml, at least about 120 mg/ml, at least about 130 mg/ml, at least about 140 mg/ml, at least about 150 mg/ml, at least about 160 mg/ml, at least about 170 mg/ml, at least about 180 mg/ml, at least about 190 mg/ml, at least about 200 mg/ml, at least about 250 mg/ml, or at least about 300 mg/ml of the anti-VISTA antibody disclosed herein.

The present compositions include a buffering or pH adjusting agent to provide improved pH control, thereby maintaining the pH in the desired range. For example, a composition as disclosed herein has a pH of between about 3.0 and about 9.0, between about 4.0 and about 8.0, between about 5.0 and about 8.0, between about 5.0 and about 7.0, between about 5.0 and about 6.5, between about 5.5 and about 8.0, between about 5.5 and about 7.0, or between about 5.5 and about 6.5. In a further embodiment, a composition of the disclosure has a pH of about 3.0, about 3.5, about 4.0, about 4.5, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.5, about 8.0, about 8.5, or about 9.0. In a specific embodiment, a composition of the disclosure has a pH of about 6.5.

Buffering agents can be present at concentration ranging from about 2 mM to about 50 mM. Preferably, the buffering agent is present at a concentration of at least 2, 5, 10, 15, 20, 25, 30, 35, 40, or 45 mM. Preferably, the buffering agent is present at a concentration of less than 45, 40, 35, 30, 25, 20, 15, 10, 5, or 2 mM. More preferably, the concentration of buffering agent is comprised between 5 and 45 mM, 10 and 40 mM, 15 and 35 mM, 20 and 30 mM. Most preferably, the concentration of buffering agent is about 25 mM.

Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium gluconate mixture, gluconic acid-sodium hydroxide mixture, gluconic acid-potassium gluconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used. Preferably, the buffering agent is selected in the group consisting of citrate buffers, phosphate buffers, and histidine buffers. More preferably, the buffering agent is a histidine buffer. More preferably, the histidine buffer is present at a concentration of 25 mM.

The skilled person will understand that the compositions disclosed herein may be isotonic with human blood, that is, the compositions have essentially the same osmotic pressure as human blood. Preferably, the osmotic pressure of the present compositions ranges from about 100 mOSm to about 1200 mOSm, or from about 200 mOSm to about 1000 mOSm, or from about 200 mOSm to about 800 mOSm, or from about 200 mOSm to about 600 mOSm, or from about 250 mOSm to about 500 mOSm, or from about 250 mOSm to about 400 mOSm, or from about 250 mOSm to about 350 mOSm. The present compositions will more preferably have an osmotic pressure from about 250 mOSm to about 350 mOSm. Isotonicity can be measured by, for example, using a vapour pressure or ice-freezing type osmometer. Tonicity of a composition is adjusted by the use of tonicity modifiers. “Tonicity modifiers” are those pharmaceutically acceptable inert substances that can be added to the composition to ensure isotonicity of liquid compositions of the present disclosure and include polyhydric sugar alcohols, for example trihydric or higher sugar alcohols, such as glycerin, erythritol, arabitol, xylitol, sorbitol and mannitol, salts and amino acids.

In certain embodiments, the compositions of the present disclosure have an osmotic pressure from about 100 mOSm to about 1200 mOSm, or from about 200 mOSm to about 1000 mOSm, or from about 200 mOSm to about 800 mOSm, or from about 200 mOSm to about 600 mOSm, or from about 250 mOSm to about 500 mOSm, or from about 250 mOSm to about 400 mOSm, or from about 250 mOSm to about 350 mOSm.

In certain embodiments, the compositions of the present disclosure have an osmotic pressure from 100 mOSm to 1200 mOSm, or from 200 mOSm to 1000 mOSm, or from 200 mOSm to 800 mOSm, or from 200 mOSm to 600 mOSm, or from 250 mOSm to 500 mOSm, or from 250 mOSm to 400 mOSm, or from 250 mOSm to 350 mOSm.

Concentration of any one or any combination of various components of the compositions described herein is adjusted to achieve the desired tonicity of the final composition. Amino acids that are pharmaceutically acceptable and suitable for this disclosure as tonicity modifiers include, but are not limited to, proline, alanine, L-arginine, asparagine, L-aspartic acid, glycine, serine, lysine, and histidine. The desired isotonicity of the final composition may notably be achieved by adjusting the salt concentration of the compositions. Salts that are pharmaceutically acceptable and suitable for this disclosure as tonicity modifiers include, but are not limited to, sodium chloride, sodium succinate, sodium sulphate, potassium chloride, magnesium chloride, magnesium sulphate, and calcium chloride. Advantageously, the present compositions comprise NaCl, MgCl₂, and/or CaCl₂). In another embodiment, concentration of MgCl₂ is between about 1 mM and about 100 mM. In one embodiment, concentration of NaCl is between about 75 mM and about 150 mM.

Preservatives can be added to retard microbial growth, and can be added in amounts ranging from 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Stabilisers refer to a broad category of excipients which can range in function from a bulking agent to an additive which solubilises the therapeutic agent (i.e., an anti-VISTA antibody, an antigen-binding fragment thereof, or a conjugate thereof) or helps to prevent denaturation or adherence to the container wall. Typical stabilisers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, α-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trisaccacharides such as raffinose; and polysaccharides such as dextran. Stabilisers can be present in the range from 0.1 to 10,000 weights per part of weight active protein (e.g., an anti-VISTA antibody or a conjugate comprising such an antibody). Preferably, the pharmaceutical composition described herein comprises at least one stabiliser selected from arginine and sucrose. Arginine may for example be present at a concentration comprised between 0 and 50 mM. in another instance, the concentration sucrose may range from 0 to 6%.

Non-ionic surfactants or detergents (also known as “wetting agents”) can be added to help solubilise the anti-VISTA antibody (or the conjugate thereof) as well as to protect the therapeutic protein against agitation-induced aggregation, which also permits the formulation to be exposed to shear surface stressed without causing denaturation of the protein. Suitable non-ionic surfactants include polysorbates (20, 80, etc.), polyoxamers (184, 188, etc.), pluronic polyols, polyoxyethylene sorbitan monoethers (TWEEN®-20, TWEEN®-80, etc.). Non-ionic surfactants can be present in a range of about 0.05 mg/ml to about 1.0 mg/ml, for example about 0.07 mg/ml to about 0.2 mg/ml. Preferably, the pharmaceutical composition described herein comprises a non-ionic surfactant which is a polysorbate, such as e.g., Polysorbate 20 or Polysorbate 80. The polysorbate may be present in the pharmaceutical composition comprised between 0 and 1%, preferably between 0 and 0.5%. Thus Polysorbate is preferably present in the pharmaceutical compositions described herein at a concentration of 0, 0.1%, 0.2%, 0.3%, 0.4%, or 0.5%.

Additional miscellaneous excipients include bulking agents (e.g., starch), chelating agents (e.g., EDTA), antioxidants (e.g., ascorbic acid, methionine, vitamin E), and cosolvents.

Preferably, the pharmaceutical composition disclosed herein comprises 25 mM Histidine, 150 mM NaCl, 0.3% Polysorbate 80 (w/w)*, pH 6.5. more preferably, this pharmaceutical composition comprises 20 mg/ml of the anti-VISTA antibody or antigen-binding fragment thereof or conjugate thereof disclosed herein.

The present disclosure is further directed to a pharmaceutical composition comprising at least:
i) an anti-VISTA antibody, an antigen-binding fragment thereof, or a conjugate thereof, as disclosed herein; and
ii) a second therapeutic agent, for example an immune checkpoint inhibitor as described below,
as combination products for simultaneous, separate, or sequential use.

“Simultaneous use” as used herein refers to the administration of the two compounds of the composition according to the invention in a single and identical pharmaceutical form.

“Separate use” as used herein refers to the administration, at the same time, of the two compounds of the composition according to the invention in distinct pharmaceutical forms.

“Sequential use” as used herein refers to the successive administration of the two compounds of the composition according to the invention, each in a distinct pharmaceutical form.

Compositions of anti-VISTA antibodies (or antigen-binding fragments thereof or conjugates thereof) and second therapeutic agents, such as e.g., immune checkpoint inhibitors, can be administered singly, as mixtures of one or more anti-VISTA antibodies (or antigen-binding fragments thereof or conjugates thereof) and/or one or more a second therapeutic agent (for example an immune checkpoint inhibitor as described below), in mixture or combination with other agents useful for treating cancer or adjunctive to other therapy for cancer. Examples of suitable combination and adjunctive therapies are provided below.

Encompassed by the present disclosure are pharmaceutical kits containing anti-VISTA antibodies (or antigen-binding fragments thereof or conjugates thereof) and described herein. The pharmaceutical kit is a package comprising an anti-VISTA antibody (e.g., either in lyophilised form or as an aqueous solution) and one or more of the following:
A second therapeutic agent, for example an immune checkpoint inhibitor as described below;
A device for administering the anti-VISTA antibody, for example a pen, needle and/or syringe; and
Pharmaceutical grade water or buffer to resuspend the antibody if the inhibitor is in antibody form.

Each unit dose of the anti-VISTA antibody (or antigen-binding fragments thereof or conjugates thereof) can be packaged separately, and a kit can contain one or more-unit doses (e.g., two-unit doses, three-unit doses, four-unit doses, five-unit doses, eight-unit doses, ten-unit doses, or more). In a specific embodiment, the one or more-unit doses are each housed in a syringe or pen.

### Effective Amounts

The anti-VISTA antibodies and conjugates thereof, optionally in combination with immune checkpoint inhibitors, will generally be used in an amount effective to achieve the intended result, for example an amount effective to treat cancer in a subject in need thereof. Pharmaceutical compositions comprising anti-VISTA antibodies (or conjugates thereof) and/or immune checkpoint inhibitors can be administered to patients (e.g., human subjects) at therapeutically effective dosages.

Determination of the effective amount is well within the capability of those skilled in the art, especially in light of the detailed disclosure provided herein. Toxicity and therapeutic efficacy of a compound or a conjugate can be determined by standard pharmaceutical procedures in cell cultures and in experimental animals. The effective amount of present combination or other therapeutic agent to be administered to a subject will depend on the stage, category and status of the disease (e.g., cancer) and characteristics of the subject, such as general health, age, sex, body weight and drug tolerance. The effective amount of the present therapeutic agent or combination to be administered will also depend on administration route and dosage form. Dosage amount and interval can be adjusted individually to provide plasma levels of the active compound that are sufficient to maintain desired therapeutic effects.

The amount of the anti-VISTA antibody or antigen-binding fragment thereof or conjugates thereof administered will depend on a variety of factors, including the nature and stage of the disease being treated (e.g., cancer), the form, route and site of administration, the therapeutic regimen (e.g., whether the therapeutic agent is used in combination with immune checkpoint inhibitors), the age and condition of the particular subject being treated, the sensitivity of the patient being treated with the antibodies or the conjugates. The appropriate dosage can be readily determined by a person skilled in the art. Ultimately, a physician will determine appropriate dosages to be used. This dosage can be repeated as often as appropriate. If side effects develop the amount and/or frequency of the dosage can be altered or reduced, in accordance with normal clinical practice. The proper dosage and treatment regimen can be established by monitoring the progress of therapy using conventional techniques known to the people skilled of the art.

Effective dosages can be estimated initially from in vitro assays. For example, an initial dose for use in animals may be formulated to achieve a circulating blood or serum concentration of anti-VISTA antibody that is at or above the binding affinity of the antibody for VISTA as measured in vitro. Calculating dosages to achieve such circulating blood or serum concentrations taking into account the bioavailability of the particular antibody is well within the capabilities of skilled artisans. For guidance, the reader is referred to Fingl & Woodbury, “General Principles” in Goodman and Gilman's The Pharmaceutical Basis of Therapeutics, Chapter 1, latest edition, Pagamonon Press, and the references cited therein. Initial dosages can be estimated from in vivo data, such as animal models. Animal models useful for testing the efficacy of compounds to treat particular diseases such as cancer are generally well known in the art. Ordinarily skilled artisans can routinely adapt such information to determine dosages suitable for human administration.

The effective dose of the anti-VISTA antibody as described herein can range from about 0.001 to about 75 mg/kg per single (e.g., bolus) administration, multiple administrations or continuous administration, or to achieve a serum concentration of 0.01-5000 μg/ml serum concentration per single (e.g., bolus) administration, multiple administrations or continuous administration, or any effective range or value therein depending on the condition being treated, the route of administration and the age, weight and condition of the subject. In a certain embodiment, each dose can range from about 0.5 μg to about 50 μg per kilogram of body weight, for example from about 3 μg to about 30 μg per kilogram body weight.

Amount, frequency, and duration of administration will depend on a variety of factors, such as the patient's age, weight, and disease condition. A therapeutic regimen for administration can continue for 2 weeks to indefinitely, for 2 weeks to 6 months, from 3 months to 5 years, from 6 months to 1 or 2 years, from 8 months to 18 months, or the like. Optionally, the therapeutic regimen provides for repeated administration, e.g., once daily, twice daily, every two days, three days, five days, one week, two weeks, or one month. The repeated administration can be at the same dose or at a different dose. The administration can be repeated once, twice, three times, four times, five times, six times, seven times, eight times, nine times, ten times, or more. A therapeutically effective amount of anti-VISTA antibody or a conjugate thereof (optionally in combination with immune checkpoint inhibitors) can be administered as a single dose or over the course of a therapeutic regimen, e.g., over the course of a week, two weeks, three weeks, one month, three months, six months, one year, or longer.

### Methods of Treatment

The anti-VISTA antibody or antigen-binding fragment thereof or conjugates thereof described herein are capable of promoting T cell activation, including T cell proliferation and cytokines production, notably through activation of the effector functions of the antibody. The anti-VISTA antibody or antigen-binding fragment thereof or conjugates thereof described herein may thus be used in methods for inducing an immune response, wherein said methods comprise administering an effective amount of an anti-VISTA antibody or a conjugate to a patient in need thereof. In particular, the present anti-VISTA antibody or antigen-binding fragment thereof or conjugates thereof is for use in inducing an immune response. The present disclosure also relates to the use of the anti-VISTA antibody or antigen-binding fragment thereof or conjugates thereof for making a medicament for inducing an immune response. In a particular embodiment of the methods described herein, the induction of the immune response requires activation of the effector functions of the antibody.

The anti-VISTA antibody or antigen-binding fragment thereof or conjugates thereof, described herein may be used in methods for inducing an immune response, wherein the induction of the immune response comprises inhibiting VISTA-mediated immunosuppression, and wherein said methods comprise administering an effective amount of an anti-VISTA antibody or a conjugate to a patient in need thereof. Preferably, the anti-VISTA antibody or antigen-binding fragment thereof or conjugates thereof, described herein may thus be used in methods for inducing an immune response, wherein the induction of the immune response comprises promoting T cell activation, and wherein said methods comprise administering an effective amount of an anti-VISTA antibody or a conjugate to a patient in need thereof. T cell activation may comprise in particular, stimulation of T cell proliferation, e.g., CD4⁺ T cell proliferation and/or CD8⁺ T cell proliferation, and/or cytokine production, notably proinflammatory cytokines, e.g., INF-γ, IL-2, and/or TNF-α.

The ability of the present anti-VISTA antibody to induce an immune response, e.g., by promoting T cell activation, notably through induction of CD4⁺ T cell proliferation, CD8⁺ T cell proliferation, CD4⁺ T cell cytokine production, and/or CD8⁺ T cell cytokine production, thereby inhibiting VISTA-mediated immunosuppression, makes it useful for treating a variety of conditions mediated by VISTA, including cancer. Therapeutic intervention on the VISTA inhibitory pathway thus represents a promising approach to modulate inflammation and T cell-mediated immunity for the treatment of a wide variety of VISTA-mediated diseases, notably cancers. Indeed, the antibody disclosed herein inhibits tumour growth in vivo.

The anti-VISTA antibody or antigen-binding fragment thereof or conjugates thereof described herein may thus be used in methods for treating VISTA-mediated diseases, notably cancer, wherein said methods comprise administering an effective amount of an anti-VISTA antibody or a conjugate to a patient in need thereof. The anti-VISTA antibody, or conjugate, described herein may thus be used in methods for treating VISTA-mediated diseases, notably cancer, wherein the treatment comprises inhibiting VISTA-mediated immunosuppression, and wherein said methods comprise administering an effective amount of an anti-VISTA antibody or a conjugate to a patient in need thereof. Preferably, the anti-VISTA antibody or antigen-binding fragment thereof or conjugates thereof, described herein may thus be used in methods for treating VISTA-mediated diseases, notably cancer, wherein the treatment comprises promoting T cell activation, and wherein said methods comprise administering an effective amount of an anti-VISTA antibody or a conjugate to a patient in need thereof.

The anti-VISTA antibody or antigen-binding fragment thereof or conjugates thereof described herein may thus be used in methods for treating VISTA-mediated diseases, notably cancer, inducing CD4⁺ T cell proliferation, inducing CD8⁺ T cell proliferation, inducing CD4⁺ T cell cytokine production, and/or inducing CD8⁺ T cell cytokine production, wherein said methods comprise administering an effective amount of an anti-VISTA antibody or a conjugate to a patient in need thereof. Preferably, the anti-VISTA antibody or antigen-binding fragment thereof or conjugates thereof described herein may thus be used in methods for treating VISTA-mediated diseases, notably cancer, wherein the treatment comprises inducing CD4⁺ T cell proliferation, inducing CD8⁺ T cell proliferation, inducing CD4⁺ T cell cytokine production, and/or inducing CD8⁺ T cell cytokine production, and wherein said methods comprise administering an effective amount of an anti-VISTA antibody or a conjugate to a patient in need thereof.

Surprisingly, effector functions are required for activation of T cell by the present antibody. In contrast, a version of the humanised IgG1 anti-VISTA mAb engineered to avoid binding to human Fcγ receptors through a N298A mutation (Herbs et al. Nature 515(7528): 563-567), therefore devoid of any effector function, is incapable of inducing either of CD4⁺ proliferation, CD8⁺ proliferation, production of CD4⁺ T cell cytokine, and production CD8⁺ T cell cytokine. Accordingly, this variant of the anti-VISTA antibody described herein is unable to inhibit tumour proliferation in vivo.

More preferably, the anti-VISTA antibody or antigen-binding fragment thereof or conjugates thereof, described herein may be used in methods for treating VISTA-mediated diseases, notably cancer, wherein the treatment comprises wherein the treatment comprises promoting T cell activation by activation of the effector functions of the antibody, and wherein said methods comprise administering an effective amount of an anti-VISTA antibody or a conjugate to a patient in need thereof. Even more preferably, the anti-VISTA antibody or antigen-binding fragment thereof or conjugates thereof, described herein may thus be used in methods for treating VISTA-mediated diseases, notably cancer, wherein the treatment comprises inducing CD4⁺ T cell proliferation, inducing CD8⁺ T cell proliferation, inducing CD4⁺ T cell cytokine production, and/or inducing CD8⁺ T cell cytokine production, by activation of the effector functions of the antibody, and wherein said methods comprise administering an effective amount of an anti-VISTA antibody or a conjugate to a patient in need thereof. The therapeutic methods described herein may comprise administration of the antibodies binding specifically VISTA described herein, or antigen-binding fragments thereof, or conjugates comprising these antibodies as disclosed herein, to a patient in need thereof. The VISTA antibodies and conjugates thereof, disclosed herein, are thus useful in regulating immunity, especially T cell immunity, for the treatment of VISTA-mediated diseases, notably cancer.

Accordingly, an aspect of the present disclosure relates to an anti-VISTA antibody or antigen-binding fragment thereof or conjugates thereof for use in the treatment of a VISTA-mediated disease, notably cancer, in a patient. Also provided herein is a method of treating a VISTA-mediated disease, notably cancer, in a patient in need thereof, said method comprising the administration of an anti-VISTA antibody, an antigen-binding fragment thereof, or a conjugate disclosed herein to the patient. The present disclosure also relates to the use of an anti-VISTA antibody or antigen-binding fragment thereof or conjugates thereof for making a medicament for treating a cancer.

In an embodiment, the disclosure relates to a composition comprising an anti-VISTA antibody disclosed herein or a conjugate thereof, for use in the treatment of a VISTA-mediated disease, notably cancer, in a patient. Also provided herein is a method of treating a VISTA-mediated disease, notably cancer, in a patient in need thereof, said method comprising the administration of a composition comprising an anti-VISTA antibody disclosed herein, or an antigen-biding fragment or a conjugate thereof, to the patient. The present disclosure also relates to the use of a composition comprising an anti-VISTA antibody disclosed herein, or an antigen-biding fragment or a conjugate thereof, for making a medicament for treating a VISTA-mediated disease, notably cancer.

Cancer that can be treated with the antibody disclosed herein can include any malignant or benign tumour of any organ or body system. Examples include, but are not limited to, the following: breast, digestive/gastrointestinal, endocrine, neuroendocrine, eye, genitourinary, germ cell, gynaecologic, head and neck, hematologic/blood, musculoskeletal, neurologic, respiratory/thoracic, bladder, colon, rectal, lung, endometrial, kidney, pancreatic, salivary gland, liver, stomach, peritoneal, testicular, oesophageal, prostate, brain, cervical, ovarian and thyroid cancers. Other cancers can include melanomas, mesothelioma, sarcomas, glioblastoma, haematological cancers such as leukaemia, myelomas, and lymphomas, and any cancer described herein. In some embodiments, the solid tumour is infiltrated with myeloid and/or T-cells. In some embodiments, the cancer is a leukaemia, lymphoma, myelodysplastic syndrome, mesothelioma, and/or myeloma. In some embodiments, the cancer can be any kind or type of leukaemia, including a lymphocytic leukaemia or a myelogenous leukaemia, such as, e.g., acute lymphoblastic leukaemia (ALL), chronic lymphocytic leukaemia (CLL), acute myeloid (myelogenous) leukaemia (AML), chronic myelogenous leukaemia (CML), hairy cell leukaemia, T-cell prolymphocytic leukaemia, large granular lymphocytic leukaemia, or adult T-cell leukaemia. In some embodiments, the lymphoma is a histocytic lymphoma, follicular lymphoma or Hodgkin lymphoma, and in some embodiments, the cancer is a multiple myeloma. In some embodiments, the cancer is a solid tumour, for example, a melanoma, or bladder cancer. In a particular embodiment, the cancer is a lung cancer, such as a non-small cell lung cancer (NSCLC). The present invention also provides a method for modulating or treating at least one malignant disease in a cell, tissue, organ, animal or patient, including, but not limited to, at least one of: leukaemia, acute leukaemia, acute lymphoblastic leukaemia (ALL), B-cell, T-cell or FAB ALL, acute myeloid leukaemia (AML), chronic myelocytic leukaemia (CML), chronic lymphocytic leukaemia (CLL), hairy cell leukaemia, myelodysplastic syndrome (MDS), a lymphoma, Hodgkin's disease, a malignant lymphoma, non-Hodgkin's lymphoma, Burkitt's lymphoma, multiple myeloma, Kaposi's sarcoma, colorectal carcinoma, pancreatic carcinoma, nasopharyngeal carcinoma, malignant histiocytosis, paraneoplastic syndrome/hypercalcemia of malignancy, solid tumours, adenocarcinomas, sarcomas, malignant melanoma, haemangioma, metastatic disease, cancer related bone resorption, cancer-related bone pain, and the like. In some embodiments, the solid tumour is infiltrated with myeloid and/or T-cells. In a particular embodiment, the solid tumour is a lung cancer, such as a non-small cell lung cancer (NSCLC). In another embodiment, the solid tumour is mesothelioma.

Preferably, the cancer is selected in the group consisting of the cancer bladder cancer, breast cancer, cervical cancer, colon cancer, endometrial cancer, oesophageal cancer, fallopian tube cancer, gall bladder cancer, gastrointestinal cancer, head-and-neck cancer, haematological cancer (e.g., leukaemia, lymphoma, or myeloma), laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma, and prostate cancer.

The present antibody is particularly useful because it can induce an immune response in a patient having a VISTA-mediated disease, e.g., a cancer patient, as detailed above. Thus, in an embodiment, the anti-VISTA antibody or antigen-binding fragment thereof or conjugates thereof is for use in the treatment of a VISTA-mediated diseases, notably cancer, in a patient, wherein the use comprises inducing an immune response in the patient. Also provided herein is a method of treating VISTA-mediated diseases, notably cancer, in a patient in need thereof, the method comprising administering the anti-VISTA antibody or a conjugate thereof disclosed herein to the patient and inducing an immune response in this patient. The present disclosure also relates to the use of an anti-VISTA antibody or antigen-binding fragment thereof or conjugates thereof for making a medicament for treating a VISTA-mediated diseases, notably cancer, wherein the treatment comprises inducing an immune response in the patient.

In an embodiment, the disclosure relates to a composition as disclosed herein, wherein the composition comprises the present anti-VISTA antibody or a conjugate thereof, for use in the treatment of a VISTA-mediated diseases, notably cancer, in a patient, wherein the use comprises inducing an immune response in the patient. Also provided herein is a method of treating VISTA-mediated diseases, notably cancer, in a patient in need thereof, the method comprising administering the anti-VISTA antibody or a conjugate disclosed herein to the patient and inducing an immune response in this patient. The present disclosure also relates to the use of a composition disclosed herein, wherein the composition comprises the present anti-VISTA antibody or a conjugate thereof, for making a medicament for treating a VISTA-mediated disease, notably cancer, wherein the treatment comprises inducing an immune response in the patient.

An embodiment provides the anti-VISTA antibody or antigen-binding fragment thereof or conjugates thereof for use in inducing an immune response in a patient having a VISTA-mediated disease, e.g., a cancer patient. Also provided herein is a method of inducing an immune response in a patient having a VISTA-mediated disease, e.g., a cancer patient, in need thereof, said method comprising the administration of the anti-VISTA antibody or a conjugate disclosed herein to the patient. The present disclosure also relates to the use of the anti-VISTA antibody or antigen-binding fragment thereof or conjugates thereof for making a medicament for inducing an immune response in a patient having a VISTA-mediated disease, e.g., a cancer patient.

In an embodiment, the disclosure relates to a composition comprising an anti-VISTA antibody disclosed herein or a conjugate thereof, for use in inducing an immune response in a patient having a VISTA-mediated disease, e.g., a cancer patient. Also provided herein is a method of an immune response in a patient having a VISTA-mediated disease, e.g., a cancer patient, in need thereof, said method comprising the administration of a composition comprising an anti-VISTA antibody disclosed herein or a conjugate thereof, to the patient. The present disclosure also relates to the use of a composition comprising an anti-VISTA antibody disclosed herein or a conjugate thereof, for making a medicament for inducing an immune response in a patient having a VISTA-mediated disease, e.g., a cancer patient.

The immune response thus generated by the antibody disclosed herein includes, without limitation, induction of CD4⁺ T cell proliferation, induction of CD8⁺ T cell proliferation, induction of CD4⁺ T cell cytokine production, and induction of CD8⁺ T cell cytokine production. Preferably, the effector functions are required for the antibody disclosed herein to generate the immune response, including, without limitation, induction of CD4⁺ T cell proliferation, induction of CD8⁺ T cell proliferation, induction of CD4⁺ T cell cytokine production, and induction of CD8⁺ T cell cytokine production.

The anti-VISTA antibody or antigen-binding fragment thereof or conjugate thereof, may be admixed with a second therapeutic agent.

A “therapeutic agent” encompasses biological agents, such as an antibody, a peptide, a protein, an enzyme, and chemotherapeutic agents. The therapeutic agent also encompasses immuno-conjugates of cell-binding agents (CBAs) and chemical compounds, such as antibody-drug conjugates (ADCs). The drug in the conjugates can be a cytotoxic agent, such as one described herein.

As used herein, the anti-VISTA antibody or antigen-binding fragment thereof or conjugate thereof, and the other therapeutic agent are said to be administered successively if they are administered to the patient on the same day, for example during the same patient visit. Successive administration can occur 1, 2, 3, 4, 5, 6, 7 or 8 hours apart. In contrast, the anti-VISTA antibody, or antigen-binding fragment or conjugate thereof, of the disclosure and the other therapeutic agent are said to be administered separately if they are administered to the patient on the different days, for example, the anti-VISTA antibody, or antigen-binding fragment or conjugate thereof, of the disclosure and the other therapeutic agent can be administered at a 1-day, 2-day or 3-day, one-week, 2-week or monthly intervals. In the methods of the present disclosure, administration of the anti-VISTA antibody, or antigen-binding fragment or conjugate thereof, of the disclosure can precede or follow administration of the other therapeutic agent.

As a non-limiting example, the anti-VISTA antibody or antigen-binding fragment thereof or conjugate thereof, and other therapeutic agent can be administered concurrently for a period of time, followed by a second period of time in which the administration of the anti-VISTA antibody, or antigen-binding fragment or conjugate thereof, of the disclosure and the other therapeutic agent is alternated.

Combination therapies of the present disclosure can result in a greater than additive, or a synergistic, effect, providing therapeutic benefits where neither the anti-VISTA antibody, or antigen-binding fragment or conjugate thereof, nor the other therapeutic agent is administered in an amount that is, alone, therapeutically effective. Thus, such agents can be administered in lower amounts, reducing the possibility and/or severity of adverse effects.

In a preferred embodiment, the other therapeutic agent is a chemotherapeutic agent. Said chemotherapeutic agent is preferably an alkylating agent, an antimetabolite, an anti-tumour antibiotic, a mitotic inhibitor, a chromatin function inhibitor, an anti-angiogenesis agent, an anti-oestrogen, an anti-androgen or an immunomodulator.

The term “alkylating agent,” as used herein, refers to any substance which can cross-link or alkylate any molecule, preferably nucleic acid (e.g., DNA), within a cell. Examples of alkylating agents include nitrogen mustard such as mechlorethamine, chlorambucol, melphalen, chlorydrate, pipobromen, prednimustin, disodic-phosphate or estramustine; oxazophorins such as cyclophosphamide, altretamine, trofosfamide, sulfofosfamide or ifosfamide; aziridines or imine-ethylènes such as thiotepa, triethylenamine or altetramine; nitrosourea such as carmustine, streptozocin, fotemustin or lomustine; alkyle-sulfonates such as busulfan, treosulfan or improsulfan; triazenes such as dacarbazine; or platinum complexes such as cis-platinum, oxaliplatin and carboplatin.

The expression “anti-metabolites,” as used herein, refers to substances that block cell growth and/or metabolism by interfering with certain activities, usually DNA synthesis. Examples of anti-metabolites include methotrexate, 5-fluoruracil, floxuridine, 5-fluorodeoxyuridine, capecitabine, cytarabine, fludarabine, cytosine arabinoside, 6-mercaptopurine (6-MP), 6-thioguanine (6-TG), chlorodesoxyadenosine, 5-azacytidine, gemcitabine, cladribine, deoxycoformycin and pentostatin.

As used herein, “anti-tumour antibiotics” are compounds which may prevent or inhibit DNA, RNA and/or protein synthesis. Examples of anti-tumour antibiotics include doxorubicin, daunorubicin, idarubicin, valrubicin, mitoxantrone, dactinomycin, mithramycin, plicamycin, mitomycin C, bleomycin, and procarbazine.

“Mitotic inhibitors,” as used herein, prevent normal progression of the cell cycle and mitosis. In general, microtubule inhibitors or taxoids such as paclitaxel and docetaxel are capable of inhibiting mitosis. Vinca alkaloid such as vinblastine, vincristine, vindesine and vinorelbine are also capable of inhibiting mitosis.

As used herein, the terms “chromatin function inhibitors” or “topoisomerase inhibitors” refer to substances which inhibit the normal function of chromatin modelling proteins such as topoisomerase I or topoisomerase II. Examples of chromatin function inhibitors include, for topoisomerase I, camptothecine and its derivatives such as topotecan or irinotecan, and, for topoisomerase II, etoposide, etoposide phosphate and teniposide.

As used herein, the term “anti-angiogenesis agent” refers to any drug, compound, substance or agent which inhibits growth of blood vessels. Exemplary anti-angiogenesis agents include, but are by no means limited to, razoxin, marimastat, batimastat, prinomastat, tanomastat, ilomastat, CGS-27023A, halofuginon, COL-3, neovastat, BMS-275291, thalidomide, CDC 501, DMXAA, L-651582, squalamine, endostatin, SU5416, SU6668, interferon-alpha, EMD121974, interleukin-12, IM862, angiostatin and vitaxin.

As used herein, the terms “anti-oestrogen” or “anti-estrogenic agent” refer to any substance which reduces, antagonizes or inhibits the action of oestrogen. Examples of anti-oestrogen agents are tamoxifen, toremifene, raloxifene, droloxifene, iodoxyfene, anastrozole, letrozole, and exemestane.

As used herein, the terms “anti-androgens” or “anti-androgen agents” refer to any substance which reduces, antagonises or inhibits the action of an androgen. Examples of anti-androgens are flutamide, nilutamide, bicalutamide, sprironolactone, cyproterone acetate, finasteride and cimitidine.

“Immunomodulators” as used herein are substances which stimulate the immune system.

Examples of immunomodulators include interferon, interleukin such as aldesleukine, OCT-43, denileukin diflitox and interleukin-2, tumoural necrose fators such as tasonermine or others immunomodulators such as lentinan, sizofiran, roquinimex, pidotimod, pegademase, thymopentine, poly I:C or levamisole in conjunction with 5-fluorouracil.

For more detail, the person of skill in the art can refer to the manual edited by the “Association Française des Enseignants de Chimie Thérapeutique” and entitled “Traité de chimie thérapeutique”, vol. 6, Médicaments antitumouraux et perspectives dans le traitement des cancers, edition TEC & DOC, 2003.

It can also be mentioned as chemical agents or cytotoxic agents, all kinase inhibitors such as, for example, gefitinib or erlotinib.

More generally, examples of suitable chemotherapeutic agents include but are not limited to 1-dehydrotestosterone, 5-fluorouracil decarbazine, 6-mercaptopurine, 6-thioguanine, actinomycin D, adriamycin, aldesleukin, alkylating agents, allopurinol sodium, altretamine, amifostine, anastrozole, anthramycin (AMC)), anti-mitotic agents, cis-dichlorodiamine platinum (II) (DDP) cisplatin), diamino dichloro platinum, anthracyclines, antibiotics, antimetabolites, asparaginase, BCG live (intravesical), betamethasone sodium phosphate and betamethasone acetate, bicalutamide, bleomycin sulfate, busulfan, calcium leucouorin, calicheamicin, capecitabine, carboplatin, lomustine (CCNU), carmustine (BSNU), Chlorambucil, Cisplatin, Cladribine, Colchicin, conjugated estrogens, Cyclophosphamide, Cyclothosphamide, Cytarabine, Cytarabine, cytochalasin B, Cytoxan, Dacarbazine, Dactinomycin, dactinomycin (formerly actinomycin), daunirubicin HCL, daunorucbicin citrate, denileukin diftitox, Dexrazoxane, Dibromomannitol, dihydroxy anthracin dione, Docetaxel, dolasetron mesylate, doxorubicin HCL, dronabinol, E. coli L-asparaginase, emetine, epoetin-α, Erwinia L-asparaginase, esterified estrogens, estradiol, estramustine phosphate sodium, ethidium bromide, ethinyl estradiol, etidronate, etoposide citrororum factor, etoposide phosphate, filgrastim, floxuridine, fluconazole, fludarabine phosphate, fluorouracil, flutamide, folinic acid, gemcitabine HCL, glucocorticoids, goserelin acetate, gramicidin D, granisetron HCL, hydroxyurea, idarubicin HCL, ifosfamide, interferon α-2b, irinotecan HCL, letrozole, leucovorin calcium, leuprolide acetate, levamisole HCL, lidocaine, lomustine, maytansinoid, mechlorethamine HCL, medroxyprogesterone acetate, megestrol acetate, melphalan HCL, mercaptipurine, mesna, methotrexate, methyltestosterone, mithramycin, mitomycin C, mitotane, mitoxantrone, nilutamide, octreotide acetate, ondansetron HCL, oxaliplatin, paclitaxel, pamidronate disodium, pentostatin, pilocarpine HCL, plimycin, polifeprosan 20 with carmustine implant, porfimer sodium, procaine, procarbazine HCL, propranolol, rituximab, sargramostim, streptozotocin, tamoxifen, taxol, tegafur, teniposide, tenoposide, testolactone, tetracaine, thioepa chlorambucil, thioguanine, thiotepa, topotecan HCL, toremifene citrate, trastuzumab, tretinoin, valrubicin, vinblastine sulfate, vincristine sulfate, and vinorelbine tartrate.

The anti-VISTA antibody, or antigen-binding fragment or conjugate thereof, disclosed herein can be administered to a patient in need of treatment for cancer receiving a combination of chemotherapeutic agents. Exemplary combinations of chemotherapeutic agents include 5-fluorouracil (5FU) in combination with leucovorin (folinic acid or LV); capecitabine, in combination with uracil (UFT) and leucovorin; tegafur in combination with uracil (UFT) and leucovorin; oxaliplatin in combination with 5FU, or in combination with capecitabine; irinotecan in combination with capecitabine, mitomycin C in combination with 5FU, irinotecan or capecitabine. Use of other combinations of chemotherapeutic agents disclosed herein is also possible.

The anti-VISTA antibody, or antigen-binding fragment or conjugate thereof, can also be combined with other therapeutic antibodies. Accordingly, therapy based on the anti-VISTA antibody, or antigen-binding fragment or conjugate thereof, disclosed herein can be combined with, or administered adjunctive to a different monoclonal antibody such as, for example, but not by way of limitation, an anti-EGFR (EGF receptor) monoclonal antibody or an anti-VEGF monoclonal antibody. Specific examples of anti-EGFR antibodies include cetuximab and panitumumab. A specific example of an anti-VEGF antibody is bevacizumab.

Notably, the therapeutic methods described herein may comprise the administration of an immune checkpoint inhibitor along with the anti-VISTA antibody, or antigen-binding fragment or conjugate thereof. The immune checkpoint inhibitor and the anti-VISTA antibody, or antigen-binding fragment or conjugate thereof may be administered simultaneously, separately, or sequentially.

As used herein, a “checkpoint inhibitor” refers to a molecule, such as e.g., a small molecule, a soluble receptor, or an antibody, which targets an immune checkpoint and blocks the function of said immune checkpoint. More specifically, a “checkpoint inhibitor” as used herein is a molecule, such as e.g., a small molecule, a soluble receptor, or an antibody, that blocks certain proteins made by some types of immune system cells, such as T cells, and some cancer cells.

In a first embodiment, the immune checkpoint inhibitor is an inhibitor of any one of CTLA-4, PDL1, PDL2, PD1, B7-H3, B7-H4, BTLA, HVEM, TIGIT, TIM3, GAL9, LAG3, PSG-L1, VSIG4, KIR, 2B4 (belongs to the CD2 family of molecules and is expressed on all NK, γδ, and memory CD8+ (αβ) T cells), CD160 (also referred to as BY55), CGEN-15049, CHK 1 and CHK2 kinases, IDO1, A2aR and any of the various B-7 family ligands.

Exemplary immune checkpoint inhibitors include anti-CTLA-4 antibody (e.g., ipilimumab), anti-LAG-3 antibody (e.g., BMS-986016), anti-B7-H3 antibody, anti-B7-H4 antibody, anti-Tim3 antibody (e.g., TSR-022, MBG453), anti-BTLA antibody, anti-KIR antibody, anti-A2aR antibody, anti CD200 antibody, anti-PD-1 antibody (e.g., pembrolizumab, nivolumab, cemiplimab, pidilizumab), anti-PD-L1 antibody (e.g., atezolizumab, avelumab, durvalumab, BMS 936559), anti-TIGIT antibody (e.g., tiragolumab, vibostolimab), anti-VSIG4 antibody, anti-CD28 antibody, anti-CD80 or -CD86 antibody, anti-B7RP1 antibody, anti-B7-H3 antibody, anti-HVEM antibody, anti-CD137 antibody (e.g., urelumab), anti-CD137L antibody, anti-OX40 (e.g., 9B12, PF-04518600, MEDI6469), anti-OX40L antibody, anti-CD40 or -CD40L antibody, anti-GAL9 antibody, anti-IL-10 antibody, fusion protein of the extracellular domain of a PD-1 ligand, e.g. PDL-1 or PD-L2, and IgG1 (e.g., AMP-224), fusion protein of the extracellular domain of a OX40 ligand, e.g. OX40L, and IgG1 (e.g., MEDI6383), IDO1 drug (e.g., epacadostat) and A2aR drug. A number of immune checkpoint inhibitors have been approved or are currently in clinical trials. Such inhibitors include ipilimumab, pembrolizumab, nivolumab, cemiplimab, pidilizumab, atezolizumab, avelumab, durvalumab, tiragolumab, vibostolimab, BMS 936559, urelumab, 9B12, PF-04518600, BMS-986016, TSR-022, MBG453, MEDI6469, MEDI6383, and epacadostat.

Examples of immune checkpoints inhibitors are listed for example in Marin-Acevedo et al., Journal of Haematology & Oncology 11: 8, 2018; Kavecansky and Pavlick, AJHO 13(2): 9-20, 2017; Wei et al., Cancer Discov 8(9): 1069-86, 2018.

Preferably, the immune checkpoint inhibitor is an inhibitor of CTLA-4, LAG-3, Tim3, PD-1, PD-L1, PSG-L1, VSIG4, CD137, OX40, or IDO1. More preferably, the immune checkpoint inhibitor is an inhibitor of PD-1 or PD-L1. Even more preferably, the immune checkpoint inhibitor is an antibody inhibiting PD-1 or an antibody inhibiting PD-L1.

Accordingly, the present disclosure preferably relates to a combination therapy of an anti-VISTA antibody or antigen-binding fragment thereof or conjugate thereof, and an anti-PD-1 antibody or an anti-PD-L1 antibody for treating a VISTA-mediated disease, notably cancer. In a first aspect, the present anti-VISTA antibody or antigen-binding fragment thereof or conjugate thereof, is for use in treating a VISTA-mediated disease, notably cancer, wherein the treatment comprises further administrating an anti-PD-1 or an anti-PD-L1 antibody. The present disclosure also relates to a method of treating a VISTA-mediated disease, notably cancer, comprising administering an effective amount of the present anti-VISTA antibody or antigen-binding fragment thereof or conjugate thereof, and an effective amount of an anti-PD-1 or anti-PD-L1 antibody to a subject in need thereof. In another aspect, the present disclosure also relates to the use of the anti-VISTA antibody or antigen-binding fragment thereof or conjugate thereof disclosed herein for making a medicament for treating a VISTA-mediated disease, notably cancer, wherein the treatment comprises administering an anti-PD-1 antibody or an anti-PD-L1 antibody.

The anti-VISTA antibody or antigen-binding fragment thereof or conjugate thereof, and the anti-PD-1 or anti-PD-L1 antibody may be administered simultaneously, separately, or sequentially.

### Methods of Diagnosis

VISTA is overexpressed in a variety of cancers, indicating that VISTA is dependable biomarker for diagnosing a cancer. Reagents such as the labelled antibodies provided herein, which bind to VISTA protein, can thus be used for diagnostic purposes to detect, diagnose, or monitor a cell proliferative disease, disorder or condition such as e.g., cancer. In another aspect, the disclosure relates to diagnostic methods comprising measuring the level of expression of VISTA to diagnose disease mediated by immune tolerance. For example, detection of high levels of VISTA expression (e.g., VISTA protein or mRNA) in a patient sample may indicate the presence of a cancer. Additionally, these diagnostic tests may be used to assign a treatment to a patient, for example by administering a VISTA antagonist based upon the detection of a high level of VISTA expression in the patient's sample.

Anti-VISTA antibodies provided herein can be used to detect VISTA or assay VISTA levels in a biological sample using classical immunohistological methods as described herein or as known to those of skill in the art (e.g., see Jalkanen et al., 1985, J. Cell. Biol. 101:976-985; and Jalkanen et al., 1987, J. Cell. Biol. 105:3087-3096). Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulphur (³⁵S), tritium (³H), indium (¹²¹In), and technetium (⁹⁹Tc); luminescent labels, such as luminol; and fluorescent labels, such as fluorescein and rhodamine, and biotin.

Thus, in a first aspect, the invention relates to an in vitro method for detecting a VISTA-mediated cancer in a subject, said method comprising the steps of:
a) contacting a biological sample of said subject with anti-VISTA antibody disclosed herein, or antigen-binding fragment thereof; and
b) detecting the binding of said antibody, or antigen-binding fragment thereof, with said biological sample.

According to the present method, the binding of the anti-VISTA antibody indicates the presence of a VISTA-mediated cancer. Preferably, the binding of the anti-VISTA antibody in immune infiltrates of the tumour microenvironment indicates the presence of a VISTA-mediated cancer.

The invention also relates to an in vitro method for detecting a VISTA-mediated cancer in a subject, said method comprising the steps of:
a) contacting a biological sample of said subject with an anti-VISTA antibody, or an antigen-binding fragment thereof; and
b) quantifying the binding of said antibody, or antigen-binding fragment thereof, with said biological sample.

According to the present method, the binding of the anti-VISTA antibody indicates the presence of a VISTA-mediated cancer. Preferably, the binding of the anti-VISTA antibody in immune infiltrates of the tumour microenvironment indicates the presence of a VISTA-mediated cancer.

As will be apparent to the skilled artisan, the level of antibody binding to VISTA may be quantified by any means known to the person of skills in the art, as detailed hereafter. Preferred methods include the use of immunoenzymatic assays, such as ELISA or ELISPOT, immunofluorescence, immunohistochemistry (IHC), radioimmunoassay (RIA), or FACS.

The quantification of step b) of the present method is a direct reflection of the level of VISTA expression in the sample, notably in immune infiltrates of the tumour microenvironment. The present method thus allows for identifying a VISTA-mediated cancer by determining the level of expression of VISTA, as described above. In a preferred embodiment, the level of expression of VISTA in said sample, notably in immune infiltrates of the tumour microenvironment, is compared to a reference level.

According to a further preferred embodiment, the invention relates to an in vitro method for detecting a VISTA-mediated cancer in a subject, said method comprising the steps of:
a) determining the level of expression of VISTA in a biological sample of said subject; and
b) comparing the level of expression of step a) with a reference level;
   wherein an increase in the assayed level of VISTA in step a) compared to the reference level is indicative of a VISTA-mediated cancer.

The invention also relates to an in vitro method for diagnosing a VISTA-mediated cancer in a subject, said method comprising the steps of:
a) determining the level of expression of VISTA in a biological sample of said subject; and
b) comparing the level of expression of step a) with a reference level;
   wherein an increase in the assayed level of VISTA in step (b) compared to the reference level is indicative of a VISTA-mediated cancer.

The expression level of VISTA is advantageously compared or measured in relation to levels in a control cell or sample also referred to as a “reference level” or “reference expression level”. “Reference level”, “reference expression level”, “control level” and “control” are used interchangeably in the specification. A “control level” means a separate baseline level measured in a comparable control cell, which is generally disease or cancer free. The said control cell may be from the same individual, since, even in a cancerous patient, the tissue which is the site of the tumour still comprises non-tumour healthy tissue. It may also originate from another individual who is normal or does not present with the same disease from which the diseased or test sample is obtained. Within the context of the present invention, the term “reference level” refers to a “control level” of expression of VISTA used to evaluate a test level of expression of VISTA in a cancer cell-containing sample of a patient. For example, when the level of VISTA in the biological sample of a patient is higher than the reference level of VISTA, the cells will be considered to have a high level of expression, or overexpression, of VISTA. The reference level can be determined by a plurality of methods. Expression levels may thus define VISTA bearing cells or alternatively the level of expression of VISTA independent of the number of cells expressing VISTA. Thus, the reference level for each patient can be prescribed by a reference ratio of VISTA, wherein the reference ratio can be determined by any of the methods for determining the reference levels described herein.

For example, the control may be a predetermined value, which can take a variety of forms. It can be a single cut-off value, such as a median or mean. The “reference level” can be a single number, equally applicable to every patient individually, or the reference level can vary, according to specific subpopulations of patients. Thus, for example, older men might have a different reference level than younger men for the same cancer, and women might have a different reference level than men for the same cancer. Alternatively, the “reference level” can be determined by measuring the level of expression of VISTA in non-oncogenic cancer cells from the same tissue as the tissue of the neoplastic cells to be tested. As well, the “reference level” might be a certain ratio of VISTA in the neoplastic cells of a patient relative to the VISTA levels in non-tumour cells within the same patient. The “reference level” can also be a level of VISTA of in vitro cultured cells, which can be manipulated to simulate tumour cells, or can be manipulated in any other manner which yields expression levels which accurately determine the reference level. On the other hand, the “reference level” can be established based upon comparative groups, such as in groups not having elevated VISTA levels and groups having elevated VISTA levels. Another example of comparative groups would be groups having a particular disease, condition or symptoms and groups without the disease. The predetermined value can be arranged, for example, where a tested population is divided equally (or unequally) into groups, such as a low-risk group, a medium-risk group and a high-risk group.

The reference level can also be determined by comparison of the level of VISTA in populations of patients having the same cancer. This can be accomplished, for example, by histogram analysis, in which an entire cohort of patients is graphically presented, wherein a first axis represents the level of VISTA, and a second axis represents the number of patients in the cohort whose tumour cells express VISTA at a given level. Two or more separate groups of patients can be determined by identification of subsets populations of the cohort which have the same or similar levels of VISTA. Determination of the reference level can then be made based on a level which best distinguishes these separate groups. A reference level also can represent the levels of two or more markers, one of which is VISTA. Two or more markers can be represented, for example, by a ratio of values for levels of each marker.

Likewise, an apparently healthy population will have a different ‘normal’ range than will have a population which is known to have a condition associated with expression of VISTA. Accordingly, the predetermined value selected may take into account the category in which an individual falls. Appropriate ranges and categories can be selected with no more than routine experimentation by those of ordinary skill in the art. By “elevated” “increased” it is meant high relative to a selected control. Typically, the control will be based on apparently healthy normal individuals in an appropriate age bracket.

It will also be understood that the controls according to the invention may be, in addition to predetermined values, samples of materials tested in parallel with the experimental materials. Examples include tissue or cells obtained at the same time from the same subject, for example, parts of a single biopsy, or parts of a single cell sample from the subject.

Preferably, the reference level of VISTA is the level of expression of VISTA in normal tissue samples (e.g., from a patient not having a VISTA-mediated cancer, or from the same patient before disease onset).

A more definitive diagnosis of a VISTA-mediated cancer may allow health professionals to employ preventative measures or aggressive treatment earlier thereby preventing the development or further progression of the VISTA-mediated cancer.

Hereinbelow, the present invention is explained in detail in view of the examples. However, the following examples are given only for exemplification of the present invention, and it is evident that the present invention is not limited to the following examples.

### EXAMPLES

### Example 1: Identification of Deamidation Sites in Ab3

The monoclonal antibody Ab3 was originally disclosed in WO2016/94837. Ab3 comprises a heavy chain of sequence SEQ ID NO:11 and a light chain of sequence SEQ ID NO:12. Bioinformatic analysis predicts that there are 2 potential deamidation sites in the light chain and 9 in the heavy chain of Ab3.

In order to investigate whether any of these sites actually undergo deamidation, Ab3 was subjected to Cationic Exchange Chromatography (CEX) as described in Goyon et al. (J Chromatogr B Analyt Technol Biomed Life Sci. 1065-1066:119-128 (2017)).

Method: CEX (pH gradient)

### Material:

Column MabPac SCX-10 4×250 mm, 10 μm (Thermo, ref: 74625)
Eluent:
   Buffer A: CX-1 pH gradient buffer A pH 5.6 (Thermo, ref: 85349)
   Buffer B: CX-1 pH gradient buffer B pH 10.2 (Thermo, ref: 85349)
   Buffers are diluted at 1/10 with MilliQ water and filtered/0.22 μm (for extemporaneous use)
   Sample preparation & method:
Sample are diluted at 1 mg/mL with MilliQ water
Injection of 20 μL diluted sample
Gradient: see Table 2.

TABLE 2
Gradient used

| Time (min) | Flow (mL/min) | % Eluent A | % Eluent B |
|---|---|---|---|
| 0 | 1 | 100 | 0 |
| 1 | 1 | 100 | 0 |
| 31 | 1 | 0 | 100 |
| 34 | 1 | 0 | 100 |
| 35 | 1 | 100 | 0 |
| 45 | 1 | 100 | 0 |

### Results

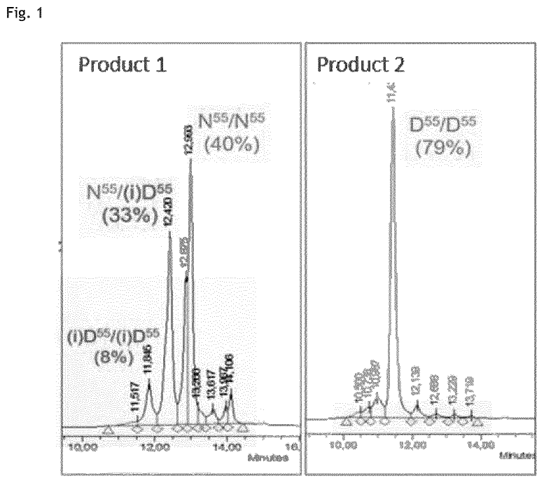

As shown on FIG. 1, Ab3 is a highly heterogenous mixture of 3 main charges variants with 40% of heavy chain N55/N55, 33% of N55 and D55 de-amidated variant, and 8% of full D55 deamidated variant. This result was confirmed by structure assessment (LC-MS). Forced degradation studies (pH9, 40° ° C., 3 days; vs pembrolizumab) led to the same conclusion: when the Asn residues of the VL and VH chains of Ab3 are examined under these conditions, degradation is only observed for N55 in the heavy chain while the other Asn residues do not appear to be affected. Therefore, in all these different experimental conditions, position N55 in the heavy chain was identified as the major, if not sole site of deamidation in Ab3.

### Example 2: Generation and Characterisation of Ab1

Based on the results of example 1, a variant of Ab3 was created by mutating the Asn at position 55 in the heavy chain into an Asp. This variant was designated Ab1.

Ab1 is an anti-VISTA humanised monoclonal antibody based on a human Immunoglobulin G1 (IgG1 k; G1m3 (R215) allotype) framework. The recombinant antibody is produced in Chinese Hamster Ovary (CHO) cells and consists of two heavy chains (HC) of 448 amino acid residues each and two kappa light chains (LC) of 213 amino acid residues each with typical IgG1 inter and intra chain disulfide bonds.

The structure, physicochemical characteristics, immunological and biological properties of anti-VISTA antibody Ab1 were established using a comprehensive set of methods:
Molecular weight: 147213 Da (GOF/GOF, pE/pE, 16 disulfide bridges)
Molecular formula: C₆₄₁₀H₉₉₀₄N₁₆₈₆O₂₀₀₉S₅₀
N-glycosylation sites: 298, 298″
Disulfide bridges are located:
   Intra-chain (light chain): Cys(23) to Cys(87); Cys(133) to Cys(193)
   Intra-chain (heavy chain): Cys(22) to Cys(96); Cys(145) to Cys(201); Cys(262) to Cys(322); Cys(368) to Cys (426)
   Inter-chain (light chain and heavy chain): Cys(213)LC to Cys(221) HC; Cys(227)HC to Cys(227)HC; Cys(230)HC to Cys(230)HC

The expected average molar mass of the full-length IgG, the deglycosylated IgG, the IdeS digested and reduced IgGs were confirmed. The N-terminal residue of the heavy chain is encoded as a glutamine but exists mainly in the pyroglutamic acid form. There is one N-glycosylation site on the heavy chain (Asn298), and it is predominantly occupied with a typical core fucosylated biantennary glycan with 0, 1 or 2 terminal galactose residues as expected for CHO produced recombinant IgGs. Most of the C-terminal lysines in the heavy chains are clipped.

The molecular weights are presented in Table 3 below:

TABLE 3
Determination of Ab1 molecular weights

Whole antibody mass (LC-UV-MS) (Da)
Expected mass: 147 213 Da
Deglycosylated antibody mass (LC-UV-MS) (Da)
Expected mass: 145 020 Da
Subunits profile, middle level (LC-UV-MS) (IdeS/reduced) (Da)
Light chain: Expected mass: 23 380 Da
Fd fragment: Expected mass: 25 024 Da
Fc/2 fragment Expected mass: 25 236 Da

### Example 3: Determination of Ab1 Binding to VISTA

Mutations in the CDR are known to affect the binding efficacy of antibodies. For example, a 400-fold decrease in antigen binding affinity was observed when Asn33 in the CRDL1 of an anti-CD52 a monoclonal antibody was replaced with an Asp (Qiu et al. mAbs. 11(7): 1266-1275 (2019)).

Ab1 binding to recombinant human (rh) VISTA-His protein VISTA was investigated by direct and indirect ELISA. In direct ELISA, the rhVISTA-His protein was directly immobilised on the plate, whilst in indirect ELISA, the rhVISTA-His protein was captured using an immobilised anti-His antibody.

Antibodies tested are provided in Table 4.

TABLE 4
Antibodies tested in ELISA

| | | Antibody |
|---|---|---|
| | | concentration |
| | | determined by |
| Antibody tested | Reference | HPLC (mg/ml) |
| Ab3 - Batch 1 (unmutated | 1109171 | 10.5 |
| antibody) | | |
| Ab3 - Batch 2 (unmutated | MID1-21 | 10.6 |
| antibody) | | |
| Ab1 (N55D substitution in | MID1-86B | 1.63 |
| the CDR-H2) | | |
| IgG1 anti-VISTA antibody | MID1-34A | 1.87 |
| with deletion | | |
| of lysine at C-terminal (positive | | |
| control) | | |
| Anti-hVISTA rabbit | SinoBio #13482-T16- | 1 |
| polyclonal | 100 | |
| antibody (positive control) | | |
| c9G4 (IgG1)/(negative | F58000.018.13094wfb | 7.45 |
| control) | | |

The anti-VISTA antibody Ab1 with an Asp at position 55 was compared with two different batches of Ab3 antibody (which has an Asn at position 55) as well as to IgG1 anti-VISTA and anti-hVISTA rabbit polyclonal antibody (positive controls) and an irrelevant c9G4 antibody (negative control).

### Direct ELISA

### Methods

Wells were coated overnight at 4° C. with 100 μl of rhVISTA at 0.3 μg/ml in 1×D-PBS.

After incubation, the coating solution was removed and plates were blocked by adding 250 μl blocking buffer (0.5% gelatin in 1×PBS) for at least 1 hour at 37° C.

After blocking, primary antibody (among those listed in Table 2) in dilution buffer (1×PBS+0.1% gelatin+0.05% Tween 20) was serially diluted 1:3 from an initial concentration of 5 μg/mL such that each well had a final volume of 100 UL and was incubated for 1 hour at 37° C.

$$Wells were washed 3× with 300 µl 1 \times P B S$$

100 μl secondary antibody (AffiniPure goat anti-rabbit specific IgG (H+L) HRP (Immuno Research Jackson ref. 111-035-003) or AffiniPure goat anti-human specific IgG (Fc fragment) HRP (Immuno Research Jackson #109-035-098), diluted 1:5000 in the dilution buffer was added to wells, and was incubated for 1 hour at 37° C.

$$Wells were washed 3× with 300 µl 1 \times P B S$$

100 μL TMB was added to each well and plates were incubated for 5 min at room temperature. Reaction was stopped with addition of 100 μl of 1 M H₂SO₄ per well and absorbance was read at 450 nm with a microplate reader.

### Results

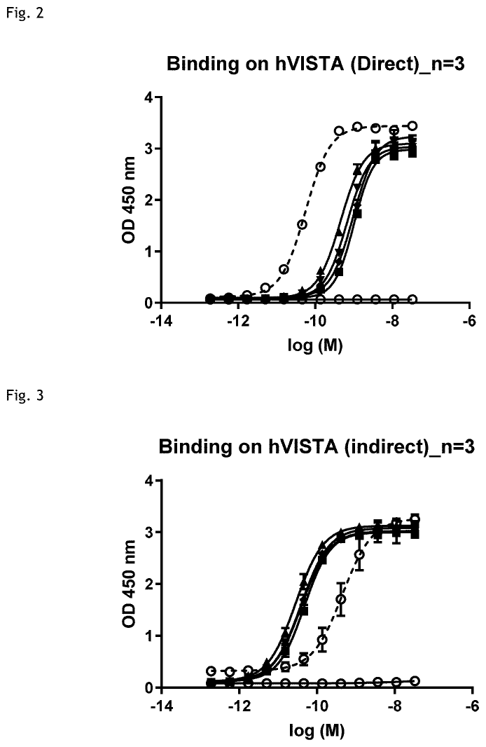

While it has been reported that several IgG1 monoclonal antibodies lose activity as a result of deamidation, binding affinity of the Ab1 anti-VISTA antibody was unexpectedly maintained. Indeed, Ab1 had a very similar profile when compared to the unmutated antibody Ab3 (see FIG. 2). EC₅₀ values were approximately 8.83×10⁻¹⁰ M (CV 21%) (Table 5), in contrast to what had previously been observed. As expected, the c9G4 negative control antibody showed no binding.

TABLE 5
EC₅₀ values obtained for the tested antibodies in each of
three experiments (n1, n2, and n3) in rhVISTA direct ELISA.

| | | | | | | | | | Mean |
|---|---|---|---|---|---|---|---|---|---|
| | Direct | | | | | STANDARD | | EC₅₀ | CV |
| | ELISA | EC₅₀ | | | MEAN EC₅₀ | DEVIATION | CV | “Prism” | “Prism” |
| | Antibody | n1 | n2 | n3 | (M) | (M) | (%) | n = 3 | (%) |
| 1 | Ab 1 | 1.04E−09 | 1.08E−09 | 9.28E−10 | 1.02E−09 | 7.92E−11 | 7.8 | 1.02E−09 | 5.9 |
| 2 | Ab 3 batch 1 | 1.10E−09 | 8.77E−10 | 9.01E−10 | 9.61E−10 | 1.25E−10 | 13.0 | 9.59E−10 | 6.9 |
| 3 | Ab 3 batch 2 | 6.41E−10 | 7.28E−10 | 6.45E−10 | 6.71E−10 | 4.93E−11 | 7.3 | 6.71E−10 | 5.2 |
| 4 | IgG1 anti-VISTA | 4.74E−10 | 4.79E−10 | 4.18E−10 | 4.57E−10 | 3.40E−11 | 7.4 | 4.58E−10 | 5.4 |
| 5 | Anti-hVISTA rabbit | 5.50E−11 | 5.17E−11 | 6.13E−11 | 5.60E−11 | 4.87E−12 | 8.7 | 5.59E−11 | 4.3 |
| | polyclonal | | | | | | | | |
| | antibody | | | | | | | | |
| | | | | MEAN | 8.83E−10 | 1.86E−10 | 21 | | |
| | | | | (1/2/3) | | | | | |

### Indirect ELISA

### Methods

Wells were coated overnight at 4° C. with 100 μl of an anti-6× Histidine mouse monoclonal IgG1, clone AD1.1.10 (RD systems cat #MAB050) at 2 μg/ml in 1×D-PBS (indirect ELISA).

After incubation, the coating solution was removed and plates were blocked by adding 250 μl blocking buffer (0.5% gelatin in 1×PBS) for at least 1 hour at 37° C.

After blocking, 100 μL of rhVISTA at 0.3 μg/ml in dilution buffer (1×PBS+0.1% gelatin+0.05% Tween 20) was added to each well and incubated for 1 hour at 37° C.

$$Wells were washed 3× with 300 µl 1 \times P B S$$

Primary antibody (among those listed in Table 2) in dilution buffer was serially diluted from an initial concentration of 5 μg/mL such that each well had a final volume of 100 μL and was incubated for 1 hour at 37° C.

$$Wells were washed 3× with 300 µl 1 \times P B S$$

100 μl secondary antibody (AffiniPure goat anti-rabbit specific IgG (H+L) HRP (Immuno Research Jackson ref. 111-035-003) or AffiniPure goat anti-human specific IgG (Fc fragment) HRP (Immuno Research Jackson #109-035-098), diluted 1:5000 in the dilution buffer was added to wells, and was incubated for 1 hour at 37° C.

$$Wells were washed 3× with 300 µl 1 \times P B S$$

100 μL TMB was added to each well and plates were incubated for 5 min at room temperature. Reaction was stopped with addition of 100 μl of 1 M H₂SO₄ per well and absorbance was read at 450 nm with a microplate reader.

### Results

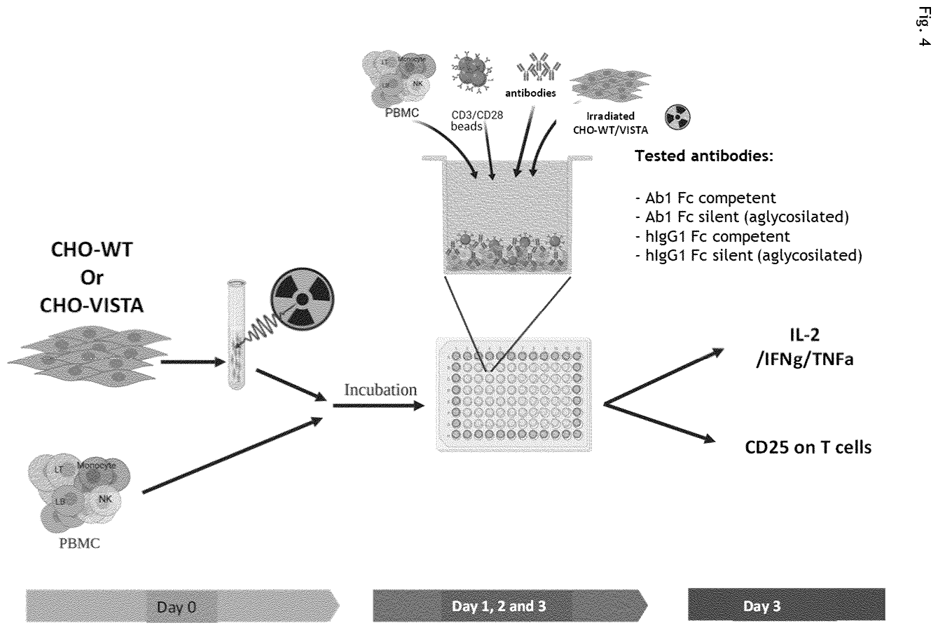

While it has been reported that several IgG1 monoclonal antibodies have reduced affinity as a result of deamidation, the affinity of the Ab1 anti-VISTA antibody was unexpectedly maintained here. Indeed, Ab1 had a very similar profile when compared to the unmutated antibody Ab3 (see FIG. 3). EC₅₀ values were approximately 4.20×10⁻¹¹ M (CV 10%) (Table 6). As expected, the c9G4 negative control antibody showed no binding.

TABLE 6
EC₅₀ values obtained for the tested antibodies in each of three
experiments (n1, n2, and n3) in rhVISTA indirect ELISA

| | | | | | | | | | Mean |
|---|---|---|---|---|---|---|---|---|---|
| | Indirect | | | | MEAN | STANDARD | | EC₅₀ | CV |
| | ELISA | EC₅₀ | | | EC₅₀ | DEVIATION | CV | “Prism” | “Prism” |
| | Antibody | n1 | n2 | n3 | (M) | (M) | (%) | n = 3 | (%) |
| 1 | Ab 1 | 5.01E−11 | 4.47E−11 | 4.65E−11 | 4.71E−11 | 2.75E−12 | 5.8 | 4.71E−11 | 6.6 |
| 2 | Ab 3 batch 1 | 4.58E−11 | 3.42E−11 | 3.86E−11 | 3.96E−11 | 5.85E−12 | 14.8 | 3.93E−11 | 6.0 |
| 3 | Ab 3 batch 2 | 3.82E−11 | 4.18E−11 | 3.80E−11 | 3.93E−11 | 2.12E−12 | 5.4 | 3.93E−11 | 3.7 |
| 4 | IgG1 anti-VISTA | 2.63E−11 | 2.59E−11 | 3.29E−11 | 2.83E−11 | 3.93E−12 | 13.9 | 2.82E−11 | 5.2 |
| 5 | Anti-hVISTA rabbit | 4.07E−10 | 3.50E−10 | 6.70E−10 | 4.76E−10 | 1.71E−10 | 35.9 | 4.53E−10 | 16.4 |
| | polyclonal | | | | | | | | |
| | antibody | | | | | | | | |
| | | | | MEAN | 4.20E−11 | 4.39E−12 | 10 | | |
| | | | | (1/2/3) | | | | | |

### Example 4: Evaluation of T Cells Activation and Cytokines Release in CHO-VISTA Coculture with PBMC

VISTA is known to be an immune checkpoint protein that critically regulates immune responses. Since Ab1 binds VISTA with the same affinity as the original antibody Ab3, it was investigated whether Ab1 was capable, like Ab3, to reverse that immune suppression.

A schematic representation of the experiment is shown in FIG. 4.

Chinese Hamster Ovary (CHO) cells WT or transfected to express human VISTA protein were irradiated with Faxitron X-ray machine 90 Gy to reduce their proliferation and metabolism.

20,000 CHO cells were then cultured with 200,000 PBMC cells in 96 well plates (ratio CHO:PBMC=1:10).

The mixture was then incubated at 37° C. and 5% CO₂ with anti-CD3/CD28 beads (ratio: 1 bead for 32 cells) in presence of Ab1 or a hIgG1 negative control 10 μg/mL in a total of 200 μl/96 wells.

At day 3, supernatants were recovered and analysed by MSD (Meso Scale Discovery) for cytokines release and by flow cytometry (FACS) for the CD25 T cells activation marker expression on CD4 and CD8 T cells.

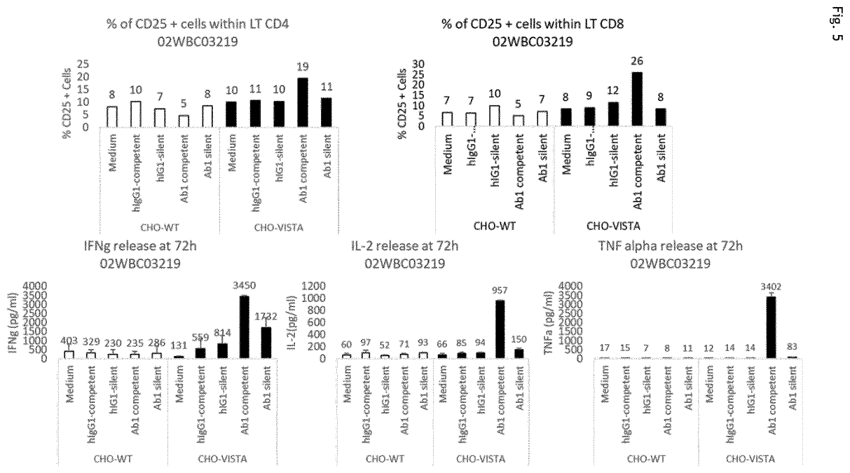

As expected, proliferation of CD4⁺ and CD8⁺ T cells was suppressed in the presence of CHO-VISTA. This suppression was reversed by the addition of the antibody Ab1. In the presence of Ab1, strong proliferation of both CD4⁺ and CD8⁺ T cells could be observed. However, no such stimulation was detected with the negative control hIgG1 antibody. Likewise, addition of Ab1 to the mixture of PBMCs and CHO-VISTA triggered a strong production of IFNγ, IL-2, and TNFα, confirming the activation of the CD4⁺ and CD8⁺ T cells (FIG. 5). Once again, the hIgG1 negative control showed no effect. These results thus demonstrate that Ab1 has retained an activity inhibiting VISTA-mediated immunosuppression.

In an attempt to understand the mechanism of this inhibition, a mutation was introduced at position 298 (N298A) in the Fc domain of Ab1. Antibodies with this mutation are known to be unable to activate effector functions, e.g., ADCC, CDC, and ADCP, as this mutation eliminates their ability to bind to human Fcγ receptors (see e.g. Liu et al. Antibodies (Basel). 9(4): 64. (2020); Herbst et al. Nature. 515(7528):563-567 (2014)).

Surprisingly, the mutation completely abolished Ab1 ability to induce CD4⁺ and CD8⁺ T cell proliferation. Likewise, no cytokine release could be detected when the Asn298 was replaced with an alanine. These results thus indicate that the interaction of Ab1 with the Fcγ receptors—and thus the effector functions of Ab1—are crucial for the Ab1 reversal of VISTA immunosuppression (FIG. 5).

The negative control was not affected by the introduction of the same mutation in its Fc.

VISTA blockade by Ab1 thus reverses immune suppression. This activity requires the effector functions (ADCC and/or CDC and/or ADCP) of the antibody.

### Example 5: Ab1 Inhibits VISTA Binding to PSG-L1 and VSIG3

Several VISTA ligands have been described. In particular, VSIG3 has been identified as a major ligand for VISTA demonstrating specific binding and functional in vitro inhibition of T cell activation (Wang et al. Immunology. 156(1):74-85 (2019)). In addition, the pH-dependent binding of VISTA to P-selectin glycoprotein ligand-1 (PSGL-1) has been described; blockade of this interaction in acidic environment is sufficient to reverse VISTA mediated immune suppression in vivo (Johnston et al. Nature. 574(7779): 565-570. (2019)).

It was therefore investigated whether Ab1 could block the interaction between VISTA and VSIG3 and/or the interaction of VISTA with PSG-L1 in an acidic pH environment (pH=6).

Evaluation of rhVISTA-his (Monomer) or rhVISTA-Fc (Dimer) Binding on rhVSIG3-Fc Grafted on a CM5 Sensor Chip (2200 RU).

The interaction was measured by Biacore at pH=7.4

rhVISTA-His (monomer) and rhVISTA-Fc (dimer) were tested at 700 nM in presence of a range of concentrations (0 to 1200 nM) of the anti-VISTA Ab1.

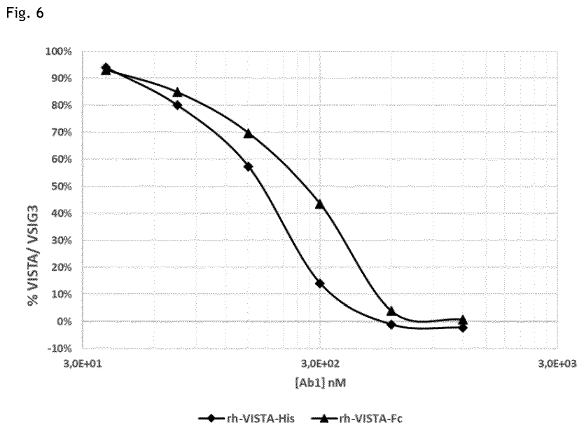

FIG. 6 shows the dose response curve of VISTA-VSIG3 binding in presence of Ab1 compared to VISTA-VSIG3 binding in absence of Ab1 (100% binding). Ab1 disrupts the VISTA-VSIG3 interaction in dose dependent manner.

### Evaluation of Anti-VISTA Antibodies on Interaction Between VISTA-Fc-d2 and PSGL1-his.

### Assay Principle:

The HTRF (Homogeneous Time-Resolved Fluorescence) technology is an assay developed to study the interaction between biomolecules. This detection system is based on a fluorescence resonance energy transfer (FRET). The interaction between hVISTA-Fc labelled with d2 (VISTA-Fc-d2) and His-tagged hPSGL-1 (PSGL1-His) bearing a Terbium-labelled anti-His mAb (Anti His-Tb/Cisbio) allows the occurrence of a HTRF signal.

The antibodies tested in the experiment were:
anti-VISTA Ab1
anti-VISTA R&D Systems #71261
human IgG1 control isotype.

The antibodies were incubated at different concentrations (0 to 20 μg/mL) during 4 hours at room temperature and at pH=6 with VISTA-Fc-d2 and PSGL1-His indirectly labelled with anti-His-Tb.

No diminution of signal is observed with the negative control. On the other hand, the addition of the positive control, i.e., commercial antibody (R&D System #71261) preventing VISTA/PSGL-1 interaction, results in the decrease of the measured HTRF signal, as expected. The IC₅₀ measured for this antibody was 333 nM (Table 7). Importantly, Ab1 also triggers a decrease of the signal, indicating the antibody blocks the interaction between VISTA and PSG-L1 at acidic pH (see Table 8). The IC₅₀ of Ab1 in VISTA/PSGL-1 HTRF interaction assay was 2.3 nM (Table 7).

TABLE 7
Anti-VISTA antibodies IC₅₀ in VISTA/PSGL-
1 HTRF interaction assay

| | IC₅₀ (nM) |
|---|---|
| Anti-VISTA (R&D Sytems) | 333 |
| Anti-VISTA Ab1 | 2.3 |

TABLE 8
Mean of 3 experiments expressed in percentage of the specific HTRF signal

| | Antibody concentration | | | | | | | | | | | | |
|---|---|---|---|---|---|---|---|---|---|---|---|---|---|
| | 0 | 0.01 | 0.02 | 0.04 | 0.08 | 0.16 | 0.31 | 0.63 | 1.25 | 2.5 | 5 | 10 | 20 |
| | μg/mL | μg/mL | μg/mL | μg/mL | μg/mL | μg/mL | μg/mL | μg/mL | μg/mL | μg/mL | μg/mL | μg/mL | μg/mL |
| hIgG1 (control | 98 | 91 | 93 | 92 | 91 | 102 | 95 | 102 | 95 | 95 | 96 | 98 | 97 |
| isotype) | | | | | | | | | | | | | |
| anti-VISTA | 96 | 100 | 106 | 99 | 96 | 89 | 88 | 81 | 68 | 63 | 50 | 34 | 21 |
| (R&D Systems) | | | | | | | | | | | | | |
| anti-VISTA Ab1 | 104 | 96 | 97 | 98 | 89 | 74 | 58 | 43 | 32 | 23 | 19 | 11 | 13 |

### Example 6: In Vivo Evaluation of Anti-VISTA mAb1 Antibody in the MC38 Murine Colon Tumour Model

### Materials and Method

For each experiment, a frozen vial of MC38 cells was thawed and grown in DMEM/F12 with 10% serum. After 2 days in culture, the cells were harvested using trypsin and resuspended in DMEM/F12 at a concentration of 5×10⁵ cells/ml and 100 μl injected per mouse.

Female C57BI/6 hVISTA mice aged 8-10 weeks were purchased from Genoway (Lyon, France). Upon arrival they were allowed to acclimatise for 7 days prior having their right flanks shaved. Mice were injected subcutananeously (s.c.) on their shaved flank, with 100 μl of MC38 cell suspension (50,000 cells).

Tumours were considered established once they reached ˜6 mm in diameter (˜80 mm³ volume). Once established, treatment was initiated. Murinised anti-VISTA antibody (mAb1), corresponding to the CDRs of Ab1 with a murine Fc, or corresponding isotype control antibody mIgG2a were administrated intraperitoneally at 30 mg/kg (formulated in Histidine 25 mM, NaCl 150 mM, 0.5% Polysorbate 80, pH 6.5), every 3 to 4 days for a total of 4 injections. Tumour growth was evaluated three times per week over the course of treatment and until the experiment was terminated, using electronic calipers across the three dimensions: length (L), width at a 90° angle to the first measurement (W) and finally height (H).

Tumour volume was derived as follows: Volume=0.52×(L×W×H)

Because T-cell activation by Ab1 was shown in vitro to be dependent on the effector functions, the role of these activities in any anti-tumour activity of the antibody was investigated. A variant of mAb1 was created in which the Asn interacting with the FcγR (the equivalent residue of N298A) in Ab1) was replaced with an Ala residue, thereby eliminating any effector mechanism. A mIgG1 antibody was used as a negative control (Chen et al. Front Immunol. 10:292 (2019).

### Results

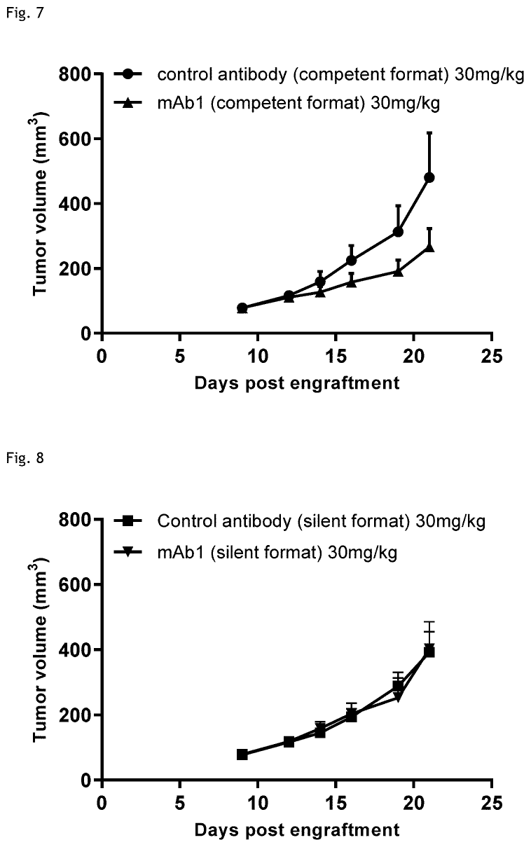

In the tested conditions of engraftment and schedule of administration, mAb1 in competent format induces a tumour growth inhibition of 47% at day 21 (FIG. 7). The silent format of mAb1 does not induce tumour growth inhibition (FIG. 8).

### Example 7: Design of a Formulation for Ab1

Formulation development, an important aspect of product development, is often on the critical path to successful clinical manufacturing and stability studies, which are essential to investigational new drug (IND) filings. Antibodies have usually been administered by infusion due notably to their large size. Because of their complex three-dimensional structures, antibodies tend to aggregate in solution, thus decreasing their shelf-life and therefore their usability.

A screening of formulation was thus implemented to select the best composition for the physicochemical stability of Ab1 bulk solution.

A pre-formulation study was performed to select four antibody formulations using a two steps approach based on experimental designs. The first step was dedicated to important factors identification and the second one to four formulations definition.

Step 1: the following parameters were evaluated:
Buffer: 25 mM Citrate or 25 mM Histidine or 25 mM Phosphate
pH: 5.5 or 6 or 6.5
Sucrose concentration: from 0 to 6% (w/v)
Arginine concentration: from 0 to 500 mM
NaCl concentrations: from 0 to 150 mM
Polysorbate 80 concentration: no Polysorbate, Polysorbate 80 0.5% or Polysorbate 20 0.5% (w/w)
the concentration of the monoclonal antibody was fixed at 20 mg/mL

Twenty-two different formulations were tested. The experimental design was set up using the MODDE software (Umetrics) which performs a statistical analysis of the data to check the validity and relevance of the generated models.

Step 2: the selected factors to be further investigated were:
Histidine buffer pH: 5.5 to 6.5
NaCl: 0 to 150 mM
Sucrose: from 0 to 6% (w/v)
Polysorbate 80: 0 to 0.5% (w/w)

The monoclonal antibodies were characterised by SEC-HPLC and Asymmetrical flow field-flow fractionation (A4FUV) to evaluate the presence of aggregates, by CEX to determine the charge variants and by differential scanning calorimetry (DSC) to determine the melting temperature (Tm).

The following formulations were selected based on the results obtained after incubation for 2 weeks and 4 weeks at 40° C. and at 5° C. and following 3 freeze/thaw cycles:
A: 25 mM Histidine, 1% Sucrose, 0.3% Polysorbate 80 (w/w)*, pH 6
B: 25 mM Histidine, 150 mM NaCl, 0.3% Polysorbate 80 (w/w)*, pH 6.5
C: 25 mM Histidine, 150 mM NaCl, 3% Sucrose, 0.3% Polysorbate 80 (w/w)*, pH 6.5
D: 25 mM Histidine, 15 mM NaCl, 5% Sucrose, 0.5% Polysorbate 80 (w/w), pH 6.5 *0.3% Polysorbate 80 (w/w) is equivalent to 0.006% v/v

A stability study was then performed with these 4 formulations with storage at −66° C., +5° C., and +40° ° C. for 1.5 month and 2 months+3 weeks. The following tests were performed: appearance, opalescence, pH, protein content by UV, SEC-HPLC, antibody purity by CE-SDS (non-reduced), charge profile by CEX, DSC, MFI and target binding by ELISA.

After 2 months 3 weeks stability study the analytical criteria for antibody quality were not discriminatory, the osmolality of the buffer was considered. Formulation A was hypotonic and on the contrary, formulation D was hypertonic. These 2 formulations were discarded.

Between formulations B and C, formulation B—i.e. 25 mM Histidine, 150 mM NaCl, 0.3% Polysorbate 80 (w/w)*, pH 6.5—was selected to limit the number of raw materials in the composition.

1. A monoclonal anti-VISTA antibody comprising a heavy chain of sequence represented by SEQ ID NO:21 and a light chain of sequence represented by SEQ ID NO:22.

2. An antibody-drug conjugate comprising the monoclonal anti-VISTA antibody of claim 1 conjugated to a cytotoxic.

3. A polynucleotide selected in the group consisting of:
a) A polynucleotide encoding the heavy chain of the monoclonal anti-VISTA antibody of claim 1,
b) A polynucleotide encoding the light chain of the monoclonal anti-VISTA antibody of claim 1, and
c) A polynucleotide encoding the heavy and the light chain of the monoclonal anti-VISTA antibody of claim 1.

4. An expression vector comprising:
a) The polynucleotide of a) and the polynucleotide of b) of claim 3; or
b) The polynucleotide of c) of claim 3.

5. A host cell comprising the expression vector of claim 4.

6. A method of producing the monoclonal anti-VISTA antibody of claim 1 comprising:
a) culturing the host cell of claim 5 under suitable conditions; and
b) recovering the anti-VISTA antibody, from the culture medium or from the cultured cells.

7. A pharmaceutical composition comprising the antibody of claim 1 or the antibody-drug conjugate of claim 2, and a pharmaceutically acceptable carrier and/or excipient.

8. The pharmaceutical composition of claim 7, comprising a buffering agent, preferably a citrate buffer, a phosphate buffer, or a histidine buffer, more preferably a histidine buffer.

9. The pharmaceutical composition of any one of claim 7 or claim 8, comprising a tonicity modifier, the tonicity modifier being preferably selected in the group consisting of polyhydric sugar alcohols, for example trihydric or higher sugar alcohols, such as glycerin, erythritol, arabitol, xylitol, sorbitol and mannitol, salts and amino acids; more preferably, a salt selected in the group consisting of sodium chloride, sodium succinate, sodium sulphate, potassium chloride, magnesium chloride, magnesium sulphate, and calcium chloride; even more preferably NaCl, MgCl₂, and/or CaCl₂).

10. The pharmaceutical composition of any one of claim 7 to claim 9, comprising a non-ionic surfactant, preferably a polysorbate, e.g., Polysorbate 20 or Polysorbate 80.

11. The pharmaceutical composition of any one of claim 7 to claim 10, comprising 25 mM Histidine, 150 mM NaCl, 0.3% Polysorbate 80 (w/w), pH 6.5.

12. The monoclonal anti-VISTA antibody of claim 1, or the immunoconjugate of claim 2, or the pharmaceutical composition of any one of claims 7 to 10, for use in the treatment of a cancer, in a patient.

13. The monoclonal anti-VISTA antibody of claim 1, or the immunoconjugate of claim 2, or the pharmaceutical composition of any one of claims 7 to 10, for the use of claim 12, wherein the use comprises inducing an immune response in the patient.

14. The monoclonal anti-VISTA antibody of claim 1, or the immunoconjugate of claim 2, or the pharmaceutical composition of any one of claims 7 to 10, for the use of claim 13, wherein the immune response includes induction of CD4⁺ T cell proliferation, induction of CD8⁺ T cell proliferation, induction of CD4⁺ T cell cytokine production, and induction of CD8⁺ T cell cytokine production.

15. The monoclonal anti-VISTA antibody of claim 1, or the immunoconjugate of claim 2, or the pharmaceutical composition of any one of claims 7 to 10, for the use of any one of claims 12 to 14, wherein the cancer is selected from bladder cancer, breast cancer, cervical cancer, colon cancer, endometrial cancer, oesophageal cancer, fallopian tube cancer, gall bladder cancer, gastrointestinal cancer, head-and-neck cancer, haematological cancer (e.g., leukaemia, lymphoma, or myeloma), laryngeal cancer, liver cancer, lung cancer, lymphoma, melanoma, mesothelioma, ovarian cancer, primary peritoneal cancer, salivary gland cancer, sarcoma, stomach cancer, thyroid cancer, pancreatic cancer, renal cell carcinoma, glioblastoma, and prostate cancer.

16. The monoclonal anti-VISTA antibody of claim 1, or the immunoconjugate of claim 2, or the pharmaceutical composition of any one of claims 7 to 10, for the use of any one of claims 12 to 15, wherein the use comprises activation of the effector functions of the antibody.

17. The monoclonal anti-VISTA antibody of claim 1, or the immunoconjugate of claim 2, or the pharmaceutical composition of any one of claims 7 to 10, for the use of any one of claims 12 to 16, further comprising the administration of a second therapeutic agent.

18. The monoclonal anti-VISTA antibody of claim 1, or the immunoconjugate of claim 2, or the pharmaceutical composition of any one of claims 7 to 10, for the use of claim 17, wherein the second therapeutic agent is an anti-PD-1 antibody or an anti-PD-L1 antibody.

19. An in vitro method for detecting a VISTA-mediated cancer in a subject, the method comprising the steps of:
a) contacting a biological sample of the subject with the monoclonal anti-VISTA antibody of claim 1; and
b) detecting the binding of the antibody with the biological sample,
wherein the binding of the anti-VISTA antibody indicates the presence of a VISTA-mediated cancer.

20. The method of claim 19, wherein the monoclonal anti-VISTA antibody is labelled with a detectable label.


SEQUENCE LISTING (22 sequences)

SEQ ID NO: 1 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 2 - DNA, 936 bp, Homo sapiens
atgggcgtccccacggccctggaggccggcagctggcgctggggatccctgctcttcgctctcttcctggctgcgtccctaggtccggtggcagccttcaaggtcgccacgccgtattccctgtatgtctgtcccgaggggcagaacgtcaccctcacctgcaggctcttgggccctgtggacaaagggcacgatgtgaccttctacaagacgtggtaccgcagctcgaggggcgaggtgcagacctgctcagagcgccggcccatccgcaacctcacgttccaggaccttcacctgcaccatggaggccaccaggctgccaacaccagccacgacctggctcagcgccacgggctggagtcggcctccgaccaccatggcaacttctccatcaccatgcgcaacctgaccctgctggatagcggcctctactgctgcctggtggtggagatcaggcaccaccactcggagcacagggtccatggtgccatggagctgcaggtgcagacaggcaaagatgcaccatccaactgtgtggtgtacccatcctcctcccaggatagtgaaaacatcacggctgcagccctggctacgggtgcctgcatcgtaggaatcctctgcctccccctcatcctgctcctggtctacaagcaaaggcaggcagcctccaaccgccgtgcccaggagctggtgcggatggacagcaacattcaagggattgaaaaccccggctttgaagcctcaccacctgcccaggggatacccgaggccaaagtcaggcaccccctgtcctatgtggcccagcggcagccttctgagtctgggcggcatctgctttcggagcccagcacccccctgtctcctccaggccccggagacgtcttcttcccatccctggaccctgtccctgactctccaaactttgaggtcatctag

SEQ ID NO: 3 - protein, 8 aa, Artificial
  CDR-H1 Ab3
GFSFTGYT

SEQ ID NO: 4 - protein, 8 aa, Artificial
  CDR-H2 Ab3
ISPYNGGT

SEQ ID NO: 5 - protein, 11 aa, Artificial
  CDR-H3 Ab3
ARRAYGYAMDY

SEQ ID NO: 6 - protein, 5 aa, Artificial
  CDR-L1 Ab3
SSVSY

SEQ ID NO: 7 - protein, 3 aa, Artificial
  CDR-L2 Ab3
DTS

SEQ ID NO: 8 - protein, 9 aa, Artificial
  CDR-L3 Ab3
QQWSSYPFT

SEQ ID NO: 9 - protein, 118 aa, Artificial
  VH Ab3
EVQLQQSGPELVKPGASMKISCKASGFSFTGYTMNWVKQSHVKNLEWIGLISPYNGGTSYNQKFKGKATLTVDKSSSTAYMELLSLTSEDSAVYYCARRAYGYAMDYWGQGTSVTVSS

SEQ ID NO: 10 - protein, 106 aa, Artificial
  VL Ab3
QIVLTQSPAIMSASPGEKVTMTCSASSSVSYMYWYQQKPGSSPRLLIYDTSNLASGVPLRFSGSGSGTSYSLTISRMEAEDAATYYCQQWSSYPFTFGSGTKLEIK

SEQ ID NO: 11 - protein, 448 aa, Artificial
  Heavy chain Ab3
QVQLVQSGAEVKKPGASVKISCKASGFSFTGYTMNWVRQAPGQGLEWIGLISPYNGGTSYAQKFQGRATLTVDTSTSTAYMELSSLRSEDTAVYYCARRAYGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 213 aa, Artificial
  Light chain Ab3
EIVLTQSPATLSLSPGERVTMSCSASSSVSYMYWYQQKPGQAPRLLIYDTSNLASGVPARFSGSGSGTDYTLTISSMEPEDFAVYYCQQWSSYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 8 aa, Artificial
  CDR-H1 Ab1
GFSFTGYT

SEQ ID NO: 14 - protein, 8 aa, Artificial
  CDR-H2 Ab1
ISPYDGGT

SEQ ID NO: 15 - protein, 11 aa, Artificial
  CDR-H3 Ab1
ARRAYGYAMDY

SEQ ID NO: 16 - protein, 5 aa, Artificial
  CDR-L1 Ab1
SSVSY

SEQ ID NO: 17 - protein, 3 aa, Artificial
  CDR-L2 Ab1
DTS

SEQ ID NO: 18 - protein, 9 aa, Artificial
  CDR-L3 Ab1
QQWSSYPFT

SEQ ID NO: 19 - protein, 118 aa, Artificial
  VH Ab1
EVQLQQSGPELVKPGASMKISCKASGFSFTGYTMNWVKQSHVKNLEWIGLISPYDGGTSYNQKFKGKATLTVDKSSSTAYMELLSLTSEDSAVYYCARRAYGYAMDYWGQGTSVTVSS

SEQ ID NO: 20 - protein, 106 aa, Artificial; identical to SEQ ID NO: 10
  VL Ab1

SEQ ID NO: 21 - protein, 448 aa, Artificial
  Heavy chain Ab1
QVQLVQSGAEVKKPGASVKISCKASGFSFTGYTMNWVRQAPGQGLEWIGLISPYDGGTSYAQKFQGRATLTVDTSTSTAYMELSSLRSEDTAVYYCARRAYGYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 22 - protein, 213 aa, Artificial; identical to SEQ ID NO: 12
  Light chain Ab1